(19) United States
(12) Patent Application Publication
(10) Pub. No.: US 2022/0235322 A1
(43) Pub. Date: Jul. 28, 2022

(54) REPRODUCIBLE BRAIN ORGANOIDS AND METHODS OF MAKING

(71) Applicants: President and Fellows of Harvard College, Cambridge, MA (US); The Broad Institute, Inc., Cambridge, MA (US)

(72) Inventors: Silvia Velasco, Moncrivello (IT); Paola Arlotta, Cambridge, MA (US)

(21) Appl. No.: 17/615,579

(22) PCT Filed: May 29, 2020
(86) PCT No.: PCT/US20/35439

§ 371 Date: Nov. 30, 2021

Related U.S. Application Data
(60) Provisional application No. 62/857,802, filed on Jun. 5, 2019.
(60) Provisional application No. 62/854,955, filed on May 30, 2019.

(51) Int. Cl. C12N 5/079; C12N 5/071; G01N 33/50

(52) U.S. Cl. CPC C12N 5/0618; C12N 5/0697; G01N 33/5058; G01N 33/5088; C12N 2501/15; C12N 2500/02; C12N 2501/727; C12N 2506/02; C12N 2506/45; C12N 2513/00; C12N 2501/415

(57) ABSTRACT

The present disclosure is directed to methods of producing dorsal forebrain organoids having cores with a very low incident of apoptotic and hypoxic cells and having highly similar cell types and cell type prevalence. The present disclosure is also directed to compositions comprising such organoids and the use of such organoids for the screening of agents.

### RELATED APPLICATION(S)

This application claims the benefit of U.S. Provisional Application No. 62/857,802, filed on Jun. 5, 2019, and U.S. Provisional Application Ser. No. 62/854,955 filed on May 30, 2019. The entire teachings of the above application are incorporated herein by reference.

### GOVERNMENT SUPPORT

This invention was made with government support under RO1-MH112940, P50MH094271, and U01MH115727 awarded by the National Institute of Health. The government has certain rights in the invention.

### BACKGROUND OF THE INVENTION

The human brain is composed of a great diversity of cell types, which are generated largely during embryonic development. In vivo, this process is virtually invariant: every embryo acquires the same compendium of cell types, organized into the same anatomical structures. It is unclear, however, whether the same reproducibility is achievable outside the embryo.

Prior studies have shown that a large variety of cell types characteristic of defined brain regions, including the cerebral cortex, can be generated in vitro, within human brain organoid and spheroid models derived from pluripotent stem cells. However, few studies have attempted to quantify cellular composition across individual organoids, limiting our understanding of the degrees of reproducibility achievable within different organoid models.

Experimental models of the human brain are needed for basic understanding of its development and disease. Human brain organoids hold unprecedented promise for this purpose; however, they are plagued by high organoid-to-organoid variability. This has raised doubts as to whether developmental processes of the human brain can occur outside the context of embryogenesis with a degree of reproducibility comparable to the endogenous tissue.

### SUMMARY OF THE INVENTION

It is shown herein that an organoid model of the dorsal forebrain (i.e. dorsal forebrain organoid) can achieve reproducible generation of a rich diversity of cell types appropriate for the human cerebral cortex. Using single-cell RNA sequencing of 166,242 cells isolated from 21 individual organoids, it is shown herein that 95% of the organoids generate a virtually indistinguishable compendium of cell types, through the same developmental trajectories, and with organoid-to-organoid variability comparable to that of individual endogenous brains. Furthermore, organoids derived from different stem cell lines show consistent reproducibility in the cell types produced. The data disclosed herein demonstrate that reproducible development of complex central nervous system cellular diversity does not require the context of the embryo, and that establishment of terminal cell identity is a highly constrained process that can emerge from diverse stem cell origins and growth environments. The findings disclosed herein are of great value for generation of reproducible organoids for study of human disease, and for screening for neurologically active agents.

Some aspects of the present disclosure are directed to a dorsal forebrain organoid having a core, wherein the core comprises less than 25% apoptotic or hypoxic cells. In some embodiments, the core comprises less than 20% apoptotic or hypoxic cells. In some embodiments, the core comprises less than 15% apoptotic or hypoxic cells. In some embodiments, the core comprises less than 10% apoptotic or hypoxic cells. In some embodiments, the core comprises less than 5% apoptotic or hypoxic cells. In some embodiments, the core comprises less than 1% apoptotic or hypoxic cells. In some embodiments, the core comprises less than 0.1% apoptotic or hypoxic cells.

In some embodiments, the organoid has been cultured for about 1-3 months. In some embodiments, the organoid (e.g., organoid cultured for 1-3 months) comprises one or more of corticofugal projection neurons, callosal projection neurons, cycling progenitors, immature corticofugal projection neurons, immature callosal projection neurons, immature projection neurons, immature interneurons, intermediate progenitor cells, outer radial glia, Cajal-Retzius neurons, and radial glia.

In some embodiments, the organoid has been cultured for about 3 months and the organoid comprises about 17%-28% corticofugal projection neurons, about 40%-50% callosal projection neurons, about 4%-7% cycling progenitors, about 2% or less (including 0%) immature interneurons, about 3%-15% immature projection neurons, about 3%-6% intermediate progenitor cells, about 9%-14% radial glia, and about 0.5% or less (including 0%) of Cajal-Retzius neurons. In some embodiments, the organoid comprises substantially no astroglia or cycling interneuron precursors.

In some embodiments, the organoid has been cultured for about 6 months or more. In some embodiments, the organoid has been cultured for about 6 months or more (e.g., about 6 months, about 9 months, about 12 months, or longer) and comprises one or more of astroglia, callosal projection neurons, cycling progenitors, immature callosal projection neurons, immature interneurons (e.g., immature inhibitory neurons), immature projection neurons, intermediate progenitor cells, outer radial glia, radial glia, ventral precursors, outer radial glia/astroglia, and cycling interneuron precursors (e.g., cycling inhibitory interneuron precursors). In some embodiments, the organoid comprises substantially no corticofugal projection neurons or immature corticofugal projection neurons.

In some embodiments, the dorsal forebrain organoid is a human dorsal forebrain organoid. In some embodiments, the dorsal forebrain organoid comprises cells having one or more mutations associated with a neurological disease or condition.

Some aspects of the disclosure are related to a method of producing a dorsal forebrain organoid, comprising obtaining a dorsal forebrain marker-positive organoid by a first step comprising culturing an aggregate of pluripotent stem cells in suspension in the presence of a Wnt signal inhibitor and a TGFβ signal inhibitor, and a second step comprising culturing the dorsal forebrain progenitor marker-positive aggregate in a spinner flask at about 20% oxygen and 5% CO₂. In some embodiments, the first step is performed for about 18 days. In some embodiments, the second step is performed for about 35 days or more. In some embodiments, the obtained dorsal forebrain organoid cultured for 1-3 months comprises one or more of corticofugal projection neurons, callosal projection neurons, cycling progenitors, immature corticofugal projection neurons, immature callosal projection neurons, immature projection neurons, intermediate progenitor cells, outer radial glia, and radial glia.

In some embodiments, the obtained dorsal forebrain organoid cultured for 6 months or more comprises one or more of astroglia, callosal projection neurons, cycling progenitors, immature callosal projection neurons, immature interneurons, immature projection neurons, intermediate progenitor cells, outer radial glia, radial glia, ventral precursors, outer radial glia/astroglia, and cycling interneuron precursors.

In some embodiments, the obtained dorsal forebrain organoid (e.g., cultured for 1, 3, 6 months or more) has a core comprising less than 25%, 20%, 15%, 10%, 5%, 1%, or 0.1% apoptotic or hypoxic cells.

In some embodiments, the first step comprises culturing the aggregate of pluripotent stem cells in suspension in the presence of a Wnt signal inhibitor and a TGFβ signal inhibitor for about 18 days and then culturing the aggregate for about 17 days without the presence of a Wnt signal inhibitor and a TGFβ signal inhibitor.

In some embodiments, the pluripotent stem cells are human pluripotent stem cells. In some embodiments, the pluripotent stem cells are derived or obtained from a subject having a neurological condition or disease.

Some aspects of the present disclosure are related to a dorsal forebrain organoid obtained by the methods disclosed herein.

Some aspects of the present disclosure are related to a method of screening for a candidate neurologically active agent, comprising contacting a dorsal forebrain organoid as described herein with a test agent, and assessing changes to the organoid, wherein the test agent is identified as a candidate neurologically active agent when contact with the test agent causes a change to the organoid as compared to a control organoid. In some embodiments, the change is a modulation of stimulus induced activity or spontaneous activity of the organoid.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIGS. 1A-1D—shows brain organoids cultured for 3 months generate cellular diversity of the human cerebral cortex with high organoid-to-organoid reproducibility. FIG. 1A—Protocol schematic. FIG. 1B—3 month PGP1 (batch 1: b1) organoids. FIG. 1C—IHC of 1 month PGP1 (b1) organoids for neuronal (MAP2) and dorsal forebrain progenitor (EMX1) markers, and of 3 month PGP1 (b1) organoids for corticofugal projection neuron (CTIP2) and callosal projection neuron (SATB2) markers. Top, entire organoids (scale bar, 200 μm); bottom, high-magnification views of three different organoids per timepoint (scale bar, 50 μm). FIG. 1D—T-distributed stochastic neighbor embedding (t-SNE) plots of scRNA-seq data from 3 month organoids after canonical correlation analysis (CCA) batch correction and alignment (PGP1: two batches, b1, b2; HUES66: one batch, n=3 organoids per batch). Left column, combined organoids from each batch, colored by cell types; right, individual organoids. Number of cells per plot are indicated. PNs, projection neurons; CPNs, callosal PNs; IPCs, intermediate progenitor cells, CFuPNs, corticofugal PNs; INs, interneurons; RG, radial glia; oRG, outer radial glia; Imm., immature; Inhib., inhibitory. Information on replicates for all figures is reported in the Methods under “Statistics and Reproducibility”.

FIGS. 2A-2C—shows brain organoids cultured for 6 months show increased cortical cell diversity while maintaining high organoid-to-organoid reproducibility. FIG. 2A—T-SNE plots of combined 6 month organoids after batch correction (11a: one batch; GM08330: one batch; PGP1: two independent batches, b1, b3; n=3 organoids per batch). Left column, combined organoids from each batch, colored by cell type; right, individual organoids. Abbreviations as in FIG. 1D. FIG. 2B—Left, t-SNE plots of PGP1 (b1) organoids at 3 and 6 months (n=3 organoids per timepoint) after batch correction. Right, percent distribution of cell types at each timepoint. FIG. 2C—IHC for astroglial markers GFAP and S100B in a PGP1 (b1) organoid at 6 months (scale bar: whole organoid, 500 μm; high-magnification, 100 μm).

FIGS. 3A-3E—shows cells in organoids are generated following a precise and reproducible trajectory and are transcriptionally similar to cells of the human fetal cortex. FIGS. 1A-1B show pseudotime t-SNE plots for the 3 (FIG. 3A) and 6 (FIG. 3B) month organoids shown in FIG. 1D and FIG. 2A, colored according to cell type (far left) and pseudotime trajectory (center left; yellow-early to green-late). Right: contribution of each batch of organoids to the pseudotime plots. (n=3 organoids per batch, downsampled to 35,000 cells per timepoint.) FIG. 3C—IHC for neuronal (MAP2) and excitatory presynaptic (VGluT1) and postsynaptic (PSD95) markers in 3 month PGP1 and 11a dorsally patterned organoids; co-localization is shown in white (arrows) (scale bars: 20 μm). Lower panels: VGluT1 and PSD95 staining alone; insets: enlargements of boxed areas. FIG. 3D—Agreement between cell type classifications in the human fetal cortex¹⁸ and in cell populations of organoids at 3 months (n=3 organoids per batch). Dot size and color intensity indicate the percent of cells in each organoid cell type assigned to each human cell type by a Random Forest classifier. Abbreviations as in FIG. 1D. FIG. 3E—Spearman correlation coefficients of variable gene expression in human fetal cortex¹⁸ with equivalent cell types found in dorsally patterned forebrain organoids (n as in FIGS. 1D and 2A) and self-patterned whole-brain organoids³ (n=19 individual organoids, 4 independent batches).

FIGS. 4A-4E show dorsally patterned organoids show sample-to-sample reproducibility similar to endogenous brains. Percent of cells from each individual organoid or endogenous cortex belonging to each cell cluster in identically processed datasets from FIG. 4A—dorsally patterned forebrain organoids at 3 months (3 batches, n=9 organoids) and 6 months (4 batches, n=11 organoids), FIG. 4B—self-patterned whole-brain organoids at 6 months³ (n=4), FIG. 4C—fetal human cortex²⁹ (n=2), FIG. 4D—adult human cortex²⁸ (n=7), and FIG. 4E—adult mouse cortex³⁰ (n=5). Top: t-SNE plots of the cell clusters for each dataset. Mutual information (MI) scores represent the dependence between the cluster and the individual (scores range from 0: individuals have the same cluster makeup, to 1: individuals cluster separately). Z-scores represent the divergence of the MI score from the mean MI score expected at random. All datasets downsampled to n=659 cells per sample.

FIGS. 5A-5B are a comparison of organoids and spheroids generated by different protocols. FIG. 5A—From left: self-patterned whole-brain organoids³, dorsally patterned forebrain organoids (protocol modified from Kadoshima et al.⁵), and dorsal and ventral forebrain spheroids (protocol modified from Rigamonti et al.¹⁵). All models are generated from the PGP1 line and cultured for 6 months. FIG. 5B—IHC for neuronal (MAP2), dorsal forebrain progenitor (EMX1 and PAX6), CFuPN (CTIP2), and CPN (SATB2) markers, across a time course from 1 to 6 months [scale bars: whole organoids (Whole Org), left column, 200 μm; close-up images, 50 μm].

FIGS. 6A-6D are an analysis of cell type-specific markers in dorsally patterned forebrain organoids derived from different lines. FIG. 6A—Expression of selected marker genes used in cell type identification. Violin plots show distribution of normalized expression in cells from CCA-aligned organoids at 3 months (n=9 organoids from 3 batches). CFuPNS: n=15,866 cells; CPNs, n=18,905 cells; Cycling, n=4,035 cells; Immature INs, n=353 cells; Immature PNs, n=6,727 cells; IPCs, 4,276 cells; oRG, n=5,436 cells; and RG, n=3,318 cells. Scale: normalized read counts. FIG. 6B—IHC for neuronal (MAP2), dorsal forebrain progenitor (EMX1 and PAX6), CFuPN (CTIP2), CPN (SATB2), radial glia (SOX2), and proliferation (Ki67) markers in PGP1 (b2), 11a, GM08330, and HUES66 organoids at 3 months. FIG. 6C—In situ RNA hybridization for the IPC (EOMES a.k.a. TBR2), Cajal-Retzius (Reelin), and post-mitotic PN (TBR1) markers in 3 month PGP1 (b2), 11a, GM08330, and HUES66 organoids. FIG. 6D—IHC for the forebrain progenitor (FOXG1), outer radial glia (HOPX), post-mitotic PN (TBR1), and IPC (TBR2) markers in PGP1 (b2) organoids at 3 and 6 months (scale bars for B, C, D: 50 μm). Abbreviations as in FIG. 1D.

FIG. 7A-7F shows an evaluation of apoptosis, hypoxia, and doublets in organoid scRNA-seq data. FIGS. 7A-7B show T-SNE plots showing average scaled expression of all genes from the FIG. 7A, apoptosis, and FIG. 7B, hypoxia, mSigDB Hallmark genesets in PGP1 (b1) organoids at 3 (left) and 6 (right) months. Abbreviations as in FIG. 1D. n=3 organoids per timepoint. FIGS. 7C-7D show histograms showing number of cells expressing FIG. 7C apoptosis markers and FIG. 7D hypoxia markers in PGP1 organoids at 3 and 6 months. X-axis indicates average scaled expression of all genes in the corresponding mSigDB Hallmark geneset. The similarity in markers of hypoxia and apoptosis between the three and six month single-cell data indicates that the growth conditions of this protocol preserve the health of the tissue over many months in culture. FIG. 7E—IHC of a 6 month PGP1 organoid for the apoptotic marker activated caspase 3, on the perimeter (Box 1) and in the center of an organoid (Box 2) (scale bar: whole organoid section, 500 μm; close-up images, 100 μm). FIG. 7F—Multiplet detection using the Scrublet³⁹ program. Scores represent the probability that the “cell” represents a droplet containing more than one cell. From left, t-SNE plots of PGP1 (two batches: b1, b2; n=3 organoids for b1, and n=3 organoids for b2) and HUES66 (n=3 organoids) organoids at 3 months; and 11a (n=3 organoids), GM08330 (n=3 organoids), and PGP1 (two batches: b1, b3; n=3 organoids for b1, and n=2 organoids for b3) organoids at 6 months. Overall, 2-8% of cells were predicted to be multiplets, which is consistent with the expected multiplet rate (˜5%) given our loadings of approximately 10,000 cells per channel³⁵.

FIGS. 8A-8B show that cell types in individual organoids are generated following a precise and reproducible temporal order and are transcriptionally similar to human fetal cortex cell types. FIG. 8A—T-SNE plots produced by Monocle2 showing the contribution of cells from individual PGP1 (b1) organoids at 3 (n=2,665, 3,094, and 2,264 cells from Orgs 1-3) and 6 months (n=3,959, 2,971, and 3,042 cells from Orgs 16-18) to plots in FIGS. 3A-3B. FIG. 8B—Agreement between cell type classifications in cell populations of 11a, GM08330, and PGP1 organoids (two batches: b1, b3) at 6 months with cell types described in a previously published single-cell RNA-seq dataset of the human fetal cortex¹⁸. Dot size and color intensity indicate the percent of organoid cells in each cell cluster assigned to each human cortex cell type by a Random Forest classifier. Abbreviations as in FIG. 1D.

FIGS. 9A-9E demonstrate that dorsally patterned forebrain organoids show reproducibility similar to that of endogenous brain, as compared to self-patterned whole-brain organoids. FIGS. 9A-9B—Percent distribution of cell types in FIG. 9A, (left) individual 3 month PGP1 (two batches: b1, b2) and HUES66 (n=3 per batch) dorsally patterned forebrain organoids, and (right) individual 6 month 11a, GM08330, and PGP1 (two batches: b1, b3, n=3 per batch) dorsally patterned forebrain organoids, vs. FIG. 9B, individual 6 month self-patterned whole-brain organoids³. FIGS. 9C-9E—Distribution of cell types as assigned in the original publication across FIG. 9C, individual samples of fetal human cortex²⁹, FIG. 9D, adult human cortex samples from distinct individuals²⁸, and FIG. 9E, adult mouse cortex samples from distinct individuals³⁰. Abbreviations as in FIG. 1D. DG, dentate gyrus; CGE, caudal ganglionic eminence; MGE, medial ganglionic eminence.

FIGS. 10A-10D show that correcting for ambient RNA contamination improves co-clustering of organoids in the 3 month HUES66 and PGP1 batch 2 datasets. FIG. 10A—Co-clustering of the three organoids in the 3 month PGP1 Batch 2 dataset before (top) and after (bottom) removal of 15 mesodermal genes identified as contributing to ambient RNA contamination from the list of variable genes used for clustering. FIG. 10B—Expression calls for the MYLPF gene in cells from Orgs 4-6 before (top) and after (bottom) ambient RNA correction. FIG. 10C—Co-clustering of the three organoids in the 3 month HUES66 dataset before (top) and after (bottom) removal of 17 genes identified as contributing to ambient RNA contamination from the list of variable genes used for clustering. FIG. 10D—Expression calls for the BASP1 gene in cells from Org 9 before (top) and after (bottom) ambient RNA correction.

FIG. 11 shows long-term culture of reproducible brain organoids as described and claimed herein. Top, schematic of the protocol for the generation of dorsal forebrain organoids. On Day 0, dissociated human induced pluripotent stem cells (iPSCs) are seeded in CDM I medium containing TGF-β and WNT inhibitors (TGF-βi, WNTi), into V-bottom 96-well plates to allow embryoid body (EB) formation. ROCK inhibitor is added from day 0 to day 6 to increase single cell survival. On Day 18, EBs are transferred to 100 mm ultra-low attachment dishes in CDM II medium. From day 35, EBs are cultured in spinner flasks containing CDM III medium, and from Day 70, CDM III is replaced with CDM IV. CDM, Cortical Differentiation Medium. Bottom, bright field images of EBs and organoids derived from the iPSC 11a line. EBs at Day 3, 9, 18, 27 and 35 were imaged by using an EVOS FL microscope (ThermoFisher Scientific; scale bar: 400 uM); organoids at day 90, by using a SMZ1500 stereoscope (Nikon; scale bar: 2 mm); and organoids at 180, by using a M60 stereoscope (Leica; scale bar: 2 mm).

FIG. 12 shows an evaluation of apoptosis across distinct organoid and spheroid models. Top, Images of 3 month organoids derived from the same iPSC line (PGP1). Bottom, Immunohistochemistry analysis of 3 month PGP1 organoids and spheroids for the apoptotic marker activated caspase 3 (CASP3). Expression of CASP3 is low in the new Dorsal forebrain organoid model, as compared to the other 3D models (Whole-brain organoids were generate according to Quadrato et al. Nature 2017; Dorsal forebrain spheroids were generated according to Rigamonti et al. Cell Reports 2016, Dorsal forebrain spheroids-mod and Ventral forebrain spheroids-mod are modifications of the Dorsal forebrain spheroid model (scale bar: 100 μm). Distance (μm) from the outer edge is indicated for each model. Activated Caspase 3 is barely detected in organoids generated with the new protocol (2), neither in their most inner core of the organoids. Other models instead show different levels of expression of this apoptotic marker.

FIG. 13 shows brain organoids cultured for 1 month generate cellular diversity of the human cerebral cortex with high organoid-to-organoid reproducibility. T-distributed stochastic neighbor embedding (t-SNE) plots of scRNA-seq data from 1 month organoids (GM08330: one batch, Mito 210: three batches, b2, b3, b4; n=3 organoids per batch). Left column, combined organoids from each batch, colored by cell types; right, individual organoids. Number of cells per plot are indicated. PNs, projection neurons; IPCs, intermediate progenitor cells; EN, excitatory neurons; RG, radial glia.

FIG. 14 shows brain organoids cultured for 3 months generate cellular diversity of the human cerebral cortex with high organoid-to-organoid reproducibility. T-distributed stochastic neighbor embedding (t-SNE) plots of scRNA-seq data from 3 month organoids (Mito 210, HUES66, and GM08330: one batch each, n=3 organoids per batch). Left column, combined organoids from each batch, colored by cell types; right, individual organoids. Number of cells per plot are indicated. PNs, projection neurons; CPNs, callosal PNs; IPCs, intermediate progenitor cells; CFuPNs, corticofugal PNs; INs, interneurons; RG, radial glia; oRG, outer radial glia; Imm., immature; Inhib., inhibitory.

### DETAILED DESCRIPTION OF THE INVENTION

Dorsal Forebrain Organoids

Some aspects of the present disclosure are directed to a dorsal forebrain organoid (also sometimes referred to herein as “dorsally patterned organoid,” “organoid,” or “DFO”) or a composition containing DFOs. As used herein, the term “organoid” refers to a three-dimensional organ-bud grown in vitro and in isolation from an intact organism. Organoids may be derived from stem cells (e.g., embryonic stem cells, induced pluripotent stem cells, etc.). In some embodiments, a dorsal forebrain organoid expresses PAX6 and MAP2 (e.g., at 1, 3, and/or 6 months of culturing). In some embodiments, the dorsal forebrain organoid express EMX1. In some embodiments, a dorsal forebrain organoid expresses EMX1, PAX6 and MAP2 (e.g., at 1, 3, and/or 6 months of culturing).

In some embodiments, the DFO has a core. In some embodiments, the core comprises the cells of the DFO that are at least 100 μM from an exterior surface of the DFO. In some embodiments, the core comprises the cells of the DFO that are at least 125 μM from an exterior surface of the DFO. In some embodiments, the core comprises the cells of the DFO that are at least 150 μM from an exterior surface of the DFO. In some embodiments, the core comprises the cells of the DFO that are at least 175 μM from an exterior surface of the DFO. In some embodiments, the core comprises the cells of the DFO that are at least 200 μM from an exterior surface of the DFO. In some embodiments, the core comprises the cells of the DFO that are at least 225 μM from an exterior surface of the DFO. In some embodiments, the core comprises the cells of the DFO that are at least 250 μM from an exterior surface of the DFO. Cells in the core tend to have reduced access to dissolved gases (e.g., oxygen) and nutrients present in culturing media. Previously, it has been found that cells located in an organoid core have a higher likelihood of apoptosis or hypoxia (e.g., expressing mSigDB hallmark geneset for apoptosis or hypoxia). However, the inventors of the present application have surprisingly discovered that the present methods, which utilizes a spinner flask for long term culture, provides organoids that have been cultured for 1 month, 3 months, or 6 months or more, exhibiting very low apoptosis or hypoxia in cells located in the core.

In some embodiments, the DFO has been cultured for 1 month and the core comprises less than about 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, 0.05%, 0.01% or less apoptotic or hypoxic cells. In some embodiments, the DFO has been cultured for 1 month and the core comprises less than about 25% apoptotic or hypoxic cells. In some embodiments, the DFO has been cultured for 1 month and the core comprises less than about 20% apoptotic or hypoxic cells. In some embodiments, the DFO has been cultured for 1 month and the core comprises less than about 15% apoptotic or hypoxic cells. In some embodiments, the DFO has been cultured for 1 month and the core comprises less than about 10% apoptotic or hypoxic cells. In some embodiments, the DFO has been cultured for 1 month and the core comprises less than about 5% apoptotic or hypoxic cells. In some embodiments, the DFO has been cultured for 1 month and the core comprises less than about 1% apoptotic or hypoxic cells. In some embodiments, the DFO has been cultured for 1 month and the core comprises less than about 0.1% apoptotic or hypoxic cells.

In some embodiments, the DFO has been cultured for 3 months and the core comprises less than about 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, 0.05%, 0.01%, or less apoptotic or hypoxic cells. In some embodiments, the DFO has been cultured for 3 months and the core comprises less than about 25% apoptotic or hypoxic cells. In some embodiments, the DFO has been cultured for 3 months and the core comprises less than about 20% apoptotic or hypoxic cells. In some embodiments, the DFO has been cultured for 3 months and the core comprises less than about 15% apoptotic or hypoxic cells. In some embodiments, the DFO has been cultured for 3 months and the core comprises less than about 10% apoptotic or hypoxic cells. In some embodiments, the DFO has been cultured for 3 months and the core comprises less than about 5% apoptotic or hypoxic cells. In some embodiments, the DFO has been cultured for 3 months and the core comprises less than about 1% apoptotic or hypoxic cells. In some embodiments, the DFO has been cultured for 3 months and the core comprises less than about 0.1% apoptotic or hypoxic cells.

In some embodiments, the DFO has been cultured for 6 months or more and the core comprises less than about 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, 0.05%, 0.01%, or less apoptotic or hypoxic cells. In some embodiments, the DFO has been cultured for 6 months and the core comprises less than about 25% apoptotic or hypoxic cells. In some embodiments, the DFO has been cultured for 6 months and the core comprises less than about 20% apoptotic or hypoxic cells. In some embodiments, the DFO has been cultured for 6 months and the core comprises less than about 15% apoptotic or hypoxic cells. In some embodiments, the DFO has been cultured for 6 months and the core comprises less than about 10% apoptotic or hypoxic cells. In some embodiments, the DFO has been cultured for 6 months and the core comprises less than about 5% apoptotic or hypoxic cells. In some embodiments, the DFO has been cultured for 6 months and the core comprises less than about 1% apoptotic or hypoxic cells. In some embodiments, the DFO has been cultured for 6 months and the core comprises less than about 0.1% apoptotic or hypoxic cells.

In some embodiments, the DFO has been cultured for 9 months or more and the core comprises less than about 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, 0.05%, 0.01%, or less apoptotic or hypoxic cells. In some embodiments, the DFO has been cultured for 9 months and the core comprises less than about 25% apoptotic or hypoxic cells. In some embodiments, the DFO has been cultured for 9 months and the core comprises less than about 20% apoptotic or hypoxic cells. In some embodiments, the DFO has been cultured for 9 months and the core comprises less than about 15% apoptotic or hypoxic cells. In some embodiments, the DFO has been cultured for 9 months and the core comprises less than about 10% apoptotic or hypoxic cells. In some embodiments, the DFO has been cultured for 9 months and the core comprises less than about 5% apoptotic or hypoxic cells. In some embodiments, the DFO has been cultured for 9 months and the core comprises less than about 1% apoptotic or hypoxic cells. In some embodiments, the DFO has been cultured for 9 months and the core comprises less than about 0.1% apoptotic or hypoxic cells.

In some embodiments, the DFO has been cultured for 1 year or more and the core comprises less than about 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, 0.05%, 0.01%, or less apoptotic or hypoxic cells. In some embodiments, the DFO has been cultured for 1 year and the core comprises less than about 25% apoptotic or hypoxic cells. In some embodiments, the DFO has been cultured for 1 year and the core comprises less than about 20% apoptotic or hypoxic cells. In some embodiments, the DFO has been cultured for 1 year and the core comprises less than about 15% apoptotic or hypoxic cells. In some embodiments, the DFO has been cultured for 1 year and the core comprises less than about 10% apoptotic or hypoxic cells. In some embodiments, the DFO has been cultured for 1 year and the core comprises less than about 5% apoptotic or hypoxic cells. In some embodiments, the DFO has been cultured for 1 year and the core comprises less than about 1% apoptotic or hypoxic cells. In some embodiments, the DFO has been cultured for 1 year and the core comprises less than about 0.1% apoptotic or hypoxic cells.

Methods of measuring the percent of apoptotic or hypoxic cells are known in the art and are not limited. In some embodiments, apoptosis and hypoxia are measured using the mSigDB hallmark geneset for apoptosis or hypoxia. In some embodiments, apoptosis is measured by detecting CASP3 (e.g., via immunohistochemistry). In some embodiments, apoptosis and hypoxia are measured using immunohistochemistry for relevant apoptosis or hypoxia markers.

In some embodiments, the DFO comprises cells expressing one or more dorsal forebrain markers, dorsal forebrain progenitor markers, early pan-neuronal markers, neuronal markers, and/or cortical markers. In some embodiments, the DFO comprises cells expressing one or more markers selected from MAP2, EMX1, PAX6, CTIP2, SATB2, SOX2, Ki67, FOXG1, HOPX, TBR1, VGluT1, PSD95, and TBR2. In some embodiments, the DFO comprises cells expressing at least 1, at least 2, at least 3, at least 4, at least 5, at least 6, at least 7, at least 8, at least 9, at least 10, at least 11, at least 12, or all 13 of these markers. In some embodiments, the DFO comprises cells expressing MAP2 and PAX6 markers. In some embodiments, the DFO comprises cells expressing MAP2, PAX6, and EMX1 markers. In some embodiments, the DFO comprises cells expressing CTIP2 and SATB2 markers. In some embodiments, the DFO comprises cells expressing MAP2, PAX6, EMX1, CTIP2, and SATB2 markers. In some embodiments, the DFO expressing the noted markers has been cultured for at least one month, at least three months, at least six months, at least 9 months, at least a year, or longer. In some embodiments, the DFO expresses one, two, or all three of TBR2, Reelin, and TBR1. In some embodiments, TBR2, Reelin, and TBR1 are detected by in situ RNA hybridization.

In some embodiments, the organoid has been cultured for about 1 month to about 3 months, and the organoid comprises one or more of corticofugal projection neurons, callosal projection neurons, cycling progenitors, immature corticofugal projection neurons, immature callosal projection neurons, immature projection neurons, immature interneurons (e.g., immature inhibitory interneurons), intermediate progenitor cells, outer radial glia, Cajal-Retzius neurons, and radial glia. In some embodiments, the organoid has been cultured for about 1 month to about 3 months, and the organoid comprises one or more of immature projection neurons, callosal projection neurons, intermediate progenitor cells, radial glia, and cycling progenitors. In some embodiments, the organoid has been cultured for about 1 month to about 3 months, and the organoid comprises immature projection neurons, callosal projection neurons, intermediate progenitor cells, radial glia, cycling progenitors, and immature interneurons.

In some embodiments, the organoid has been cultured for about 1 month to about 3 months, and the organoid comprises about 17%-28% corticofugal projection neurons. In some embodiments, the organoid has been cultured for about 1 month to about 3 months, and the organoid comprises about 40%-50% callosal projection neurons. In some embodiments, the organoid has been cultured for about 1 month to about 3 months, and the organoid comprises about 4%-7% cycling progenitors. In some embodiments, the organoid has been cultured for about 1 month to about 3 months, and the organoid comprises about 2% or less (including 0%) immature interneurons. In some embodiments, the organoid has been cultured for about 1 month to about 3 months, and the organoid comprises about 3%-15% immature projection neurons. In some embodiments, the organoid has been cultured for about 1 month to about 3 months, and the organoid comprises about 3%-6% intermediate progenitor cells. In some embodiments, the organoid has been cultured for about 1 month to about 3 months, and the organoid comprises about 9%-14% radial glia. In some embodiments, the organoid has been cultured for about 1 month to about 3 months, and the organoid comprises about 0.5% or less (including 0%) of Cajal-Retzius neurons.

In some embodiments, the organoid has been cultured for about 1 month and the organoid comprises about 17%-28% corticofugal projection neurons, about 40%-50% callosal projection neurons, about 4%-7% cycling progenitors, about 2% or less (including 0%) immature interneurons, about 3%-15% immature projection neurons, about 3%-6% intermediate progenitor cells, about 9%-14% radial glia, and about 0.5% or less (including 0%) of Cajal-Retzius neurons.

In some embodiments, the organoid has been cultured for about 3 months and the organoid comprises about 17%-28% corticofugal projection neurons, about 40%-50% callosal projection neurons, about 4%-7% cycling progenitors, about 2% or less (including 0%) immature interneurons, about 3%-15% immature projection neurons, about 3%-6% intermediate progenitor cells, about 9%-14% radial glia, and about 0.5% or less (including 0%) of Cajal-Retzius neurons. In some embodiments, the organoid has been cultured for about 1 month to about 3 months and comprises substantially no astroglia or cycling interneuron precursors. In some embodiments, the organoid has been cultured for about 1 month to about 3 months and substantially no astroglia, immature interneurons, or cycling interneuron precursors.

In some embodiments of the organoids described herein, an immature projection neuron in an organoid cultured for about 3 months is characterized as an organoid cell that overexpresses, as compared to the rest of the organoid cells, at least about the first 5, 10, 20, 30, 40, 50, 75, or all 85 of the following genes: BASP1, TUBB2B, MAP1B, TUBA1A, MLLT11, PCSK1N, PGK1, GAP43, CRMP1, HILPDA, CD24, ARMCX3, TAGLN3, NRN1, MARCKS, UCHL1, GSTA4, ENO2, STMN4, HMP19, TMSB15A, APP, TMEM132A, NCAM1, HES4, NCALD, GPR162, RUNX1T1, RCN1, INA, GPC2, EGR1, KCNQ1OT1, FAM213A, DNER, NEFL, MYL6, CADM3, SCG2, MIAT, CLU, NDN, ATF3, TM7SF2, CHGA, LRRN3, CXXC5, ETFB, SYP, KLC1, LDHA, RCN2, SCG5, CHD4, GNG3, ID4, ANK3, CNTNAP2, ARMCX1, NOVA1, APLP1, ARID5B, RNF5, LGALS3BP, MAP6, CA11, INSM1, CELF4, TMEM14C, OLFM1, FAM57B, CITED2, HACD3, BLCAP, ISYNA1, LSAMP, MDK, SYT5, AP1S2, RSRC1, BSDC1, DUT, SLF1, SEMA6A, and CHD7.

In some embodiments of the organoids described herein, an immature callosal projection neuron in an organoid cultured for about 3 months is characterized as an organoid cell that overexpresses, as compared to the rest of the organoid cells, at least about the first 5, 10, 20, 30, 40, 50, or all 55 of the following genes: SOX11, SLA, CLMP, ARHGAP21, TCF4, MT-ND3, GADD45G, FNBP1L, MEIS2, DCX, NFIB, MIAT, CADM2, ARL4C, MN1, DDAH2, LINC01102, TPGS2, CHD3, RND3, TTC28, MEX3B, DNER, GSE1, C14orf132, DPYSL4, NEDD4L, FAM60A, NUP93, RERE, SERINC5, TMSB15A, AUTS2, STARD4-AS1, MUM1, LIMD2, PHLDA1, FLRT2, KCNQ2, SERP2, SUN2, PLXNA4, ZNF300, RNF182, LRRC7, ZNF195, BAZ2B, PLPPR5, HS3ST1, ACOT7, INHBA, ZNF627, EPHA4, CAMK2B, and INSM1.

In some embodiments of the organoids described herein, a callosal projection neuron in an organoid cultured for about 3 months is characterized as an organoid cell that overexpresses, as compared to the rest of the organoid cells, at least about the first 5, 10, 20, 30, 40, 50, 75, 100, 125, 150, 175, 200, or all 237 of the following genes: EXOC4, GPR85, STMN2, INHBA, RNF182, NELL2, NEUROD6, SATB2, MEF2C, NHSL1, SNX7, SERPINI1, NREP, NCALD, NEUROD2, CAMKV, BHLHE22, DCX, DACT1, HSPA8, BASP1, MCUR1, CD24, FABP7, RTN4, FAM49A, NEFM, RAB3A, PLXNA4, INA, OLFM1, PTPN2, MT-CO2, MAP1B, GNAI1, MN1, DEAF1, PRKACB, MT-ATP6, PKIA, PEBP1, NSG1, NCAM1, SRGAP1, MAPT, RASL11B, SHTN1, ZEB2, FATS, TUBA1A, RAC3, ATAT1, DSTN, TMEM14A, JAKMIP1, RBFOX2, CRMP1, LRRC7, PPFIA2, ATP1A3, ST3GAL1, SLC8A1, MYT1L, CSRNP3, STMN4, TSPO, SCD5, SQLE, PAK7, CAMK2B, ATP2B1, ADCY1, COTL1, MT-CYB, SYBU, NUDT3, CSRP2, GFOD1, ELAVL3, TMEM160, HMGCS1, PIK3R1, AKAP7, CHCHD6, MPPED1, CDK5R1, AP3S1, GDAP1L1, DPYSL3, BCL7A, DNER, GNG3, DUSP23, APLP1, MEAF6, NAV1, PTPRD, ANK2, ANKRD46, SBK1, MMD, PHACTR3, NME1, BOP1, ADD2, MAP4, CTXN1, GNAO1, C20orf27, RAP1GDS1, HS3ST1, SH3GL3, STARD4-AS1, NOL4, SPTAN1, TMEM35, PCLO, SMAP2, AMN1, CELF3, MAP4K4, SSBP4, C2orf80, TBC1D14, RBFOX1, CHGB, PARP6, STRBP, RGS17, GRIN2B, KLHL8, ATP1B1, JPH4, SERP2, FKBP1A, MYCBP2, HMGCR, EML1, MT-ND5, PLPPR5, FARP1, FLRT2, PGD, LRRN3, NEO1, ACTN2, ATP6V0E2, FOXP1, ACAT2, CELF1, DAB1, MAPRE3, SPIN1, RRM2B, LDB2, TUSC3, ZWILCH, FAM84A, SV2A, PWAR6, ODF2L, PRKCZ, CMIP, PPP1R14C, RUNDC3B, FSD1, PSD3, ELOVL6, PAK1, RUNDC3A, CACNG8, SRD5A1, GRIA1, RP11-490M8.1, NPB, RNF219, TUBB4A, NLRP1, SSX2IP, HIVEP2, RP11-660L16.2, HSD11B1L, GFOD2, AFF3, SEC61A2, JAKMIP2, UBE2E3, BEX5, SYT5, TSPYL1, ARHGEF9, MAPRE2, PTPRO, FASN, GNAZ, HOMER1, STC1, FAM127A, RUNX1T1, DYRK2, BIVM, FBXL2, PSD, ELMO1, ATP9A, DLG2, LINC01503, TCEAL7, TMEM150C, SCG2, SNN, BOLA3-AS1, HEBP2, MGLL, ARHGAP33, MT-ND4L, CCDC184, DDX25, MYO5A, CCSAP, BAD, RASGRP2, FBXL15, BRINP1, LYPD1, SNX32, KATNB1, MASP1, ROGDI, DACH2, B4GALNT1, TCEAL1, RPRM, PDE4DIP, PGP, ULK3, and CHN2.

In some embodiments of the organoids described herein, an immature corticofugal projection neuron in an organoid cultured for about 3 months is characterized as an organoid cell that overexpresses, as compared to the rest of the organoid cells, at least about the first 5, 10, 20, 30, 40, 50, 75, 100, 125, 150, 175, 200, 250, 300, 350, 400 or all 416 of the following genes: CTA-29F11.1, RNF165, ILF3-AS1, ZNF436-AS1, RP11-51J9.5, IER5, PRR7, BRD2, ATP6V0B, RP11-356J5.12, GPR22, RP1-39G22.7, MLLT4-AS1, RRAGA, EID2B, RP4-798A10.7, BBC3, RP11-352M15.2, NAA38, VAMP2, RFK, GABPB1-AS1, NCBP2-AS2, NSMCE3, PDRG1, FBXL15, RP11-395G23.3, TAF7, POP7, HIST3H2A, TMA7, SNHG7, ZNF830, RP11-1094M14.11, IMP3, SPINT2, H1F0, BLOC1S4, MAPKAPK5-AS1, SAC3D1, MESP1, KCNQ1OT1, LINC00526, SNHG15, TRMT10C, HIST1H1C, EPC1, PHLDA3, FBXW7, PSMG3, CSTF3, EPM2AIP1, PET117, EPB41L4A-AS1, C16orf91, LINC00685, AMD1, NEFL, MAGEH1, AC093323.3, TXNIP, KBTBD7, MOAP1, MED19, BLOC1S2, EFNA3, MRPL34, PCF11, RAB33A, RP11-410L14.2, C19orf53, RP11-660L16.2, NAP1L3, C19orf25, C9orf78, NR2F6, NGDN, RP11-792A8.4, MRPL44, CHD2, PPID, ARPC5L, GSPT2, TUSC2, CAMLG, PEX13, ACYP1, POLR1C, DLL3, CDKN2AIPNL, UQCC3, DGUOK, F12, TBCC, C15orf61, PFDN2, ATG101, SLC16A1-AS1, SCNM1, LINC01315, SCOC, SLBP, TRIM32, EMC6, TAF9, TSC22D3, MRFAP1L1, TCEAL5, NPM3, LINC01006, ANKRD54, LINC01560, SELM, ZNF821, NUDC, IMP4, DOHH, RGS2, ALDOA, INTS6, C11orf71, ZSCAN16-AS1, RNF113A, HIST1H2BG, PITHD1, NFKBIA, COX17, IMMP1L, ERV3-1, CHAMP1, DDX24, CYCS, TMEM11, FAM103A1, PRKAG2-AS1, TMEM251, TYW5, PPFIA3, BOLA3-AS1, TIMM17A, FEM1A, RBM4, HIRIP3, SRSF8, LINC00662, PLK3, ZCCHC7, RDH14, ATP5G1, EIF4A2, MAGEF1, OAT, DACH2, RRS1, CCDC184, TIGD1, ASB8, CDKN2D, THAP2, UTP3, C6orf120, ZNF622, IP6K2, THAP9-AS1, EIF2B2, TM2D3, ATXN3, NDUFAF4, ZNF281, WDR74, MRPL32, CNOT8, RASL10A, PPP1R8, MKRN1, DPM3, ANKRA2, KBTBD6, PTS, SNHG8, RNASEH1, CHMP1B, GLRX5, SPIN2B, PRRT1, RCHY1, CTSL, SNAP47, CFAP20, MPHOSPH10, BOLA1, LARP6, PAK1IP1, TIPRL, TRAPPC4, ZFAS1, TMED9, HIST2H2BE, ZNF574, FAM110A, WBP5, PPP4R2, NRBF2, AHSA1, C12orf73, RP9, NUDCD2, THAP11, C2orf69, C1orf35, CCDC115, LYPLA2, ALAS1, RP11-83A24.2, TMEM167B, THAP5, LINC00667, PELO, GTF2B, TSPYL2, MEDT, PCYOX1, CNPPD1, SNX10, CSGALNACT2, GRPEL1, ING2, FUT11, PRPF4, RBM22, PPP1R2, SURF6, WBP11, SURF2, THAP3, TAF12, MED6, ZBTB43, KIAA0907, RANBP6, SAMD8, SS18L2, SDHAF1, LINC01003, C17orf58, CDKN2AIP, DUSP12, ZNF791, SDHAF2, TMEM55B, TMUB1, MAD2L1BP, BEX5, TAF1D, CCDC51, ZFPL1, ARRDC3, PDK1, CBR1, CDC37L1, MPHOSPH8, ELOVL4, PRPF38A, PPM1A, ZNF397, DAXX, ADPRHL2, ING1, MMADHC, EBP, METTL2A, RPA2, DUSP18, MRPL10, TOPORS, MAP9, G3BP2, FUCA1, MRPL49, CMBL, SIKE1, TMEM87A, TMEM183A, FKBP7, CEP57, AAR2, NXT1, RNF41, RASSF1, ATP6V1G2, PNRC2, BAGS, SCO1, DNTTIP2, RBM4B, SIRT6, CITED2, SLC39A1, CLN5, MRPS14, CWC25, LRRC59, NABP2, FDFT1, DDX21, TTC9C, P4HB, TMEM205, GGNBP2, TMEM199, CCND3, TMEM70, SCAMP3, FTSJ2, ZNF667-AS1, PARP2, ZNF131, DIS3, YIPF4, EIF2B5, PI4 KB, STIM2, LETMD1, THUMPD1, HIST1H2AC, RNF4, CLK4, ZNF274, SIRT7, CDK19, KANSL2, SEC11C, CEBPZ, NECAP1, CLK1, ZCCHC10, EED, GSKIP, FRG1, CSTF1, CCDC130, TAF13, ZMAT3, CDC40, PDCD2L, TCTN3, DEXI, C1orf174, AKT1S1, PIM3, GOT1, RNF13, C1orf109, ELP5, BRIX1, SLC35A4, RIOK2, RPL39L, FEN1, FEM1B, ZNF430, DYRK4, NKIRAS2, ELOVL6, EBLN3, ANKRD49, TMED3, GORASP2, NBR1, POLE3, PREB, DEDD2, USP15, SUN1, TRAPPC2P1, NUP50, FAM126B, CTDSPL2, C22orf29, MTHFD2, NOLC1, YIPF5, OSER1, MUL1, HSD17B7, CCDC174, VCPKMT, PDP1, AKAP17A, DNAJB4, RGS16, GEMIN2, CRIPT, CXXC1, CCP110, GPN3, RAB39B, RBBP5, ZNF581, C1orf131, BNIP1, CXorf40B, ZNF331, TNRC6C, RPP30, PRKAB1, RFC4, GAR1, ARID3A, ANKRD37, TMEM136, PIM1, PNO1, MYNN, MPPE1, and UTP6.

In some embodiments of the organoids described herein, a corticofugal projection neuron in an organoid cultured for about 3 months is characterized as an organoid cell that overexpresses, as compared to the rest of the organoid cells, at least about the first 5, 10, 20, 30, 40, 50, 75, 100, 125, 150, 175, 200, 250, or all 273 of the following genes: KAZN, PDE1A, GPR22, ETV1, FEZF2, IGSF21, BRINP1, TLE4, CELF4, SNAP25, CTNND2, SYT1, SCD5, SSBP2, OLFM1, NELL2, CXADR, MAP1B, MAPT, NBEA, VAMP2, RALYL, GRIA2, SPINT2, HMGCS1, NFIA, CPE, VRK1, PBX1, NEUROD2, RBFOX2, RBFOX1, DYNC1I1, LINC00461, SQLE, LINGO1, AGAP1, NFIB, DOK6, RP11-356J5.12, SLC26A4-AS1, KIDINS220, RTN4, PPP3CB, PPP3CA, SEZ6, INA, SESN3, CLSTN1, ITSN1, PNMA1, TMEM108, RFK, PHACTR3, DPP6, FKBP1B, PRKACB, SHTN1, NLGN1, CCDC107, NDN, MSRA, TMEM35, NSMF, TUSC3, JAKMIP2, APP, SULT4A1, FXYD6, RGS17, GNAO1, EFNA3, ANKS1B, H1F0, GABPB1-AS1, SCAMPI, NETO2, RP11-660L16.2, IER5, DCLK1, BCL11A, KIF3A, RAB33A, ITFG1, DEAF1, RPRM, NTRK3, DSEL, REEP1, H2AFJ, NFIX, ENOPH1, PRR7, NCAM1, SRRM4, ANKMY2, SCAI, WIPF3, DACH2, PHYHIP, RASL10A, DUSP5, PSD3, STT3B, ARL6IP5, GALNT11, ARL4D, CAMK2G, KCNQ1OT1, F12, SESTD1, RP11-25K19.1, HK1, FDFT1, TNIK, CMB9-22P13.1, JAKMIP1, TMEM132A, IDS, ENO2, SH3KBP1, KIFAP3, BZW2, NOV, CCDC184, CEP19, THSD7A, KCTD13, MOAP1, GTF2H5, CAMK1, SLC25A4, TMEM63B, IDH3G, CADPS, PRNP, C14orf1, DKK3, CDC40, DBP, FABP5, ALAS1, CADM1, STXBP1, LINC00685, CELF5, MYCBP2, LINC00632, PSMD1, WAC-AS1, ARL2, MT-ND6, PPID, CITED2, PNMAL1, IDH1, DAB1, ING2, THOC3, TRIB1, ROGDI, FASN, PICALM, ABR, SEMA3A, ACAP1, POLR3GL, LGALSL, PFKFB3, MESDC1, ACLY, ATP13A2, RP11-511P7.5, POMGNT2, POLD2, SLC16A1-AS1, TIPRL, BOLA3-AS1, FARSB, RP1-39G22.7, INTS12, ELOVL6, SEC61A2, GRIA3, PIGP, CYFIP2, GAL3ST3, THAP5, MYO5A, NFASC, RNF41, DNAJC12, CRK, TRIM32, RP11-68606.2, DUSP18, RUVBL1, IGF2BP2, PITHD1, PDHA1, AC093323.3, PGK1, OXCT1, ENHO, KIFC2, PCSK7, SDCCAG8, TMEM106B, FGF13, GNB5, THAP1, COQ7, SCAPER, CA11, CDK19, G2E3, MCTS1, LINC00936, GSK3B, TRIM9, GPR137, AP001372.2, MAP1A, PPCS, POLR3K, GFOD2, RAD50, ING1, PCAT6, PRPSAP2, EID2B, RP11-127B20.2, TMEM121, ACOT13, CYB5D2, C6orf136, LINC00094, PTDSS1, DZIP3, CTC-241N9.1, HDHD3, TAF12, EPB41L3, CCP110, ZNF529, EBLN3, PKIB, ARRDC1-AS1, RP11-83A24.2, PTPRF, TTPAL, IFT22, ADAL, TNNI3, LRRC49, TRAM1L1, ABHD8, PAXIP1-AS1, FAM220A, ERRFI1, MECR, COQ3, STK16, MYLIP, KBTBD4, RP6-65G23.3, SPIN2B, RP11-115C21.2, TMEM5, TNIP1, RNASEH1-AS1, CUTC, and NIT2.

In some embodiments of the organoids described herein, an intermediate progenitor cell in an organoid cultured for about 3 months is characterized as an organoid cell that overexpresses, as compared to the rest of the organoid cells, at least about the first 5, 10, 20, 30, 40, 50, 75, 100, 125, 150, or all 167 of the following genes: NFIA, PRDX1, MARCKS, SOX4, CALD1, CORO1C, HMGN2, C1orf6l, SSTR2, TMEM123, PAX6, CMC1, UBE2E3, EEF1D, SOX11, SYNE2, EZR, H3F3B, RPS6, ZBTB20, HLA-A, RCN2, AP1S2, NAP1L1, PHLDA1, B2M, MEIS2, TMEM98, PGAP1, MDK, SRSF6, TFDP2, ITGB1, MYO6, HPCAL1, NKAIN3, ROBO2, KCNQ2, GLTSCR2, SORBS2, LYPD1, BAZ2B, ADGRG1, CCND2, MDFI, MPST, CXXC5, RND3, STK17A, GADD45G, NR2F1, TCF4, MRPL42, HDAC9, MSI1, GOLIM4, RBPJ, FZD3, POU3F2, SPAG9, PGRMC2, RPS27L, BBX, HLA-B, DECR1, PRKX, MLLT4, BICD1, EBPL, USP3, HLA-C, BTG1, PHYHIPL, MSI2, TMX1, NME4, H2AFV, ASCL1, PNRC1, FYN, ATP6V0E1, BTG2, TANK, FEM1C, SKA2, FAM60A, NRN1, SEPT9, PDXK, CNN2, JAM2, PNKD, TBL1XR1, DBN1, CDK4, PNRC2, FBLN1, PTTG1IP, BAZ1A, DHRS7, KDM1A, DSEL, REC8, IFI27L2, SERINC2, C14orf132, EHBP1, DNAJC4, EZH2, LIMD2, GLUL, SMARCA5, NUDT5, GCA, USP47, RAB13, LEPROT, NFIC, LIMS1, CBFA2T2, AAMDC, CPLX2, ROCK1, AMOTL2, HADHB, LHX2, SETBP1, CHGA, TSPAN6, FOXN3, TMTC4, LMNB1, ACTL6A, POU3F3, CNR1, EMX2, RPA1, MARCH1, NDUFA7, CLIC1, BTG3, MESDC2, CLMP, ALDH7A1, TRIM24, ECI2, GNG4, HMG20B, LIMA1, TMPO, FUBP3, PAG1, SZRD1, ZFAND3, TLE3, LITAF, DAP, DDR1, PAM, FRMD4A, RIT1, MAPK10, STAT3, TECPR1, MEST, MIR124-2HG, and CNTNAP2.

In some embodiments of the organoids described herein, an radial glia in an organoid cultured for about 3 months is characterized as an organoid cell that overexpresses, as compared to the rest of the organoid cells, at least about the first 5, 10, 20, 30, 40, 50, 75, 100, 125, 150, 175, 200, or all 226 of the following genes: VIM, FTH1, BNIP3, FTL, GAPDH, ENO1, EIF1, CD9, SLC3A2, CLU, SOX2, DDIT3, NEAT1, RCN1, CD63, TCEA1, HSPB1, IGFBP2, MT2A, GADD45A, TGIF1, RPS27L, ALDOA, RPL41, SERPINH1, ANXA5, ADM, BCAN, RPL36, PHGDH, RPS20, SHMT2, PSAT1, SLC16A1, ZFP36L1, PGK1, CD99, P4HA1, SYPL1, SAT1, HSPA5, ATF4, RPS27, CXCR4, HES1, NFE2L2, CCNG1, SERPINE2, GNB2L1, SLC16A3, RGS16, HSD17B14, DARS, TPT1, RPL30, BLVRB, ATF3, SDCBP, FAM162A, HILPDA, TTYH1, EEF1D, DDIT4, PON2, SOX9, VEGFA, ATRAID, NPC2, SLC2A3, CD164, EMP3, PDLIM4, PNRC1, TMEM123, CANX, MT1X, RPL21, WSB1, LITAF, BTG3, HOPX, CTSD, GNG5, RP11-395G23.3, SCD, CRYAB, PGM1, DNAJC1, HADHB, QKI, ATP6V0E1, CSTB, GPT2, P4HB, BTG2, RHOC, CNN3, PAX6, BTG1, MID1IP1, TMEM47, XBP1, KCNG1, ID4, CALR, GPI, EMX2, NOV, PPT1, ST13, NTSC, HERPUD1, DNAJB9, ACADVL, PHYH, VKORC1, SPTSSA, ILK, MALAT1, SPG20, PRDX4, CEBPG, ADGRG1, EMD, CYR61, ITM2C, SRI, HLA-A, RPL22L1, ANKRD37, CIB1, TRIM9, B2M, HLA-B, TSC22D4, JAM2, MTHFD2, RPS16, PFKP, HLA-C, SSR3, GLUL, TMEM38B, ETV1, MIF, MYL12A, GBAS, CLNS1A, LMNA, EGLN3, PIM3, SNX2, ACAA2, CYBA, FERMT2, NGLY1, FOS, CNIH1, SNX5, FUBP3, CRYL1, SERF2, ALDH3A2, TAGLN2, GOLIM4, EPHX1, TSPAN6, TRAM1, SRA1, MESDC2, ACTN1, ETV5, ITGB1, TXNRD1, ZFAND3, AK2, PTTG1IP, CFAP36, SERP1, CHPT1, PDIA6, GCSH, ECI2, IRF2BP2, LDHA, BBX, PPIB, RHOA, RNF187, TMED7, SELK, SEPT2, LAPTM4B, ARL6IP6, CMTM6, PDIA4, EGR1, UBXN4, PAICS, CDK2AP2, C5orf28, PEX2, RAB13, RER1, ANP32B, GPX1, KDSR, TULP3, FAM84A, HBP1, FXR1, BAGS, GHITM, TMEM179B, RAB9A, SPNS1, DNPEP, RAP1A, TMEM230, TMEM263, MIF4GD, USO1, HIST1H1C, NHSL2, TMEM14C, ARRDC3, and TMX1.

In some embodiments of the organoids described herein, an outer radial glia in an organoid cultured for about 3 months is characterized as an organoid cell that overexpresses, as compared to the rest of the organoid cells, at least about the first 5, 10, 20, 30, 40, 50, 75, 100, 125, 150, 175, 200, 250, 300, or all 308 of the following genes: GFAP, ID3, HOPX, BCAN, PON2, SPARC, CLU, ID4, HES1, SOX2, PTN, ZFP36L1, TTYH1, SOX9, SCRG1, CST3, LRRC3B, DBI, RHOC, QKI, PEA15, DDAH1, SFRP1, VIM, HSPB1, ANXA5, C1orf6l, GPM6B, CNN3, SH3BGRL, HMGN3, B2M, FABP7, SRI, CD63, CKB, LIMA1, GNG5, NCAN, TAGLN2, CRYAB, LITAF, MT2A, PTPRZ1, SEPT9, PSAT1, GSTP1, PAX6, ITM2C, SEPT2, RCN1, SERF2, CD9, RPS27L, NDRG2, RHOA, ANXA6, EMP3, CYBA, PDLIM4, EZR, TSC22D4, SAT1, TMEM98, TGIF1, IFI6, GLUL, TMEM123, AP1S2, NME4, SYNE2, NFE2L2, MDK, MYL6, PHLDA1, DECR1, HADHB, CALD1, DNAJC1, NPC2, DKK3, PFN1, EEF1D, SDCBP, TMEM47, CAMTA1, ECI2, SPTSSA, C1orf122, RPS6, PPDPF, PSME1, POLR2L, CLIC1, SLC35F1, NTSC, DOK5, SEPT11, DNPH1, GPC4, MSI1, LINC00998, PDLIM7, TSPAN6, TSPAN3, SYPL1, HES4, RAB13, CCDC109B, H2AFV, PHGDH, MYL12A, SLC25A26, GBAS, ITGB1, PCBD1, SNX5, BAALC, C12orf75, PRDX6, AAMDC, PGM1, DHRS7, NKAIN3, PHYHIPL, ZBTB20, ID1, CRYL1, HMGN2, SLC25A6, MDFI, NDUFA11, ACAA2, TRIM9, HEY1, ABCD3, TMA7, TMEM132B, ADGRG1, OST4, FEZ2, CSTB, GOLIM4, ALDH7A1, FERMT2, BLOC1S1, NAP1L1, MAGED2, RDX, PXMP2, RCN2, PEX2, CD164, ATP6V0E1, CLNS1A, CXXC5, CDK4, C17orf89, ASPH, DDR1, PGLS, REEP3, ALDH9A1, KLHDC8A, HDDC2, DCXR, EFNB1, PTTG1, LHX2, C7orf50, FUBP3, EMX2, BTG3, NDUFA13, ARL6IP6, ADK, CNP, GOLM1, HIBCH, KTN1, GNAS, SEC11A, HMGN1, PSME2, HMG20B, MCL1, GPX1, KIAA0101, COMT, ACADVL, PTTG1IP, BBX, RP3-525N10.2, PHIP, SNX17, NUDT4, ROBO1, PLEKHO1, GCA, URM1, NUDT5, CD151, EGR1, HAT1, RNASEH2C, PPP1CA, UBE2E1, MGMT, CTNNBIP1, SCCPDH, POLR2J, ACTN1, APOA1BP, ILK, AKR7A2, PDIA6, ASCL1, TMEM230, PNKD, CHCHD10, TXNRD1, HADHA, LMNA, EIF2AK2, NME3, KLF6, ACADM, ETFA, CFL2, GPSM2, IDH2, JUNB, PDCD4, SMC4, NEAT1, PMF1, RHOBTB3, GADD45A, ANP32B, ABAT, HSD17B12, ZFAND3, CLDND1, TMBIM4, PEPD, TIMP2, RAB9A, DBNL, COMMD4, UQCC3, ROMO1, WDR1, TCF25, SESN3, COA4, NUTF2, UBXN4, MIF4GD, BLVRB, SNRPD3, MPPED2, C11orf3l, MMP24-AS1, NRCAM, PAICS, AHCY, COPRS, SHISA4, ANGPTL4, CNTFR, PHYH, NFIC, PRCP, CTSD, WDR6, KLHL5, SMDT1, TLK1, NDRG4, GPT2, SMARCA1, ADSL, FKBP3, RNF130, CTSL, CTBP2, SRSF2, MRPL23, CYB5R3, HADH, PRSS23, REPS1, CNDP2, DGCR6L, ALDH3A2, JPX, SERINC2, LRRFIP1, REPIN1, AC004556.1, HYI, LTBP3, ENSA, EHBP1, LYPLAL1, MCMI, TYMS, and NASP.

In some embodiments of the organoids described herein, a cycling progenitor in an organoid cultured for about 3 months is characterized as an organoid cell that overexpresses, as compared to the rest of the organoid cells, at least about the first 5, 10, 20, 30, 40, 50, 75, 100, 125, 150, 175, 200, 250, 300, 350, 400, 450, or all 472 of the following genes: PTTG1, KIAA0101, HMGB2, SMC4, H2AFX, CKAP2, CENPW, CKS1B, CKS2, HMGN2, SOX2, TUBA1B, H2AFV, UBE2T, UBE2S, HMGB3, TUBB4B, HMGB1, CKB, HSPB1, PHGDH, HNRNPA2B1, KIF22, SFRP1, DHFR, HMGN3, PTN, KPNA2, PAX6, KIF20B, CENPH, LMNB1, GNG5, MZT2B, EZH2, B2M, ANP32B, NME4, DBI, ANXA5, CLIC1, RANBP1, GPSM2, RAN, CD99, VIM, SYNE2, DUT, TAGLN2, IDH2, TMEM98, NKAIN3, DCXR, HES1, SFPQ, KNSTRN, GSTP1, FBLN1, QKI, PXMP2, SRSF2, TGIF1, WDR34, RNASEH2A, EEF1D, LITAF, RDX, HOPX, C12orf75, ID4, SNRPB, RAB13, HADH, ZFP36L1, RHOA, PON2, CLU, TMSB15A, COX8A, GPX4, GINS2, TMEM106C, EZR, SCRG1, SPARC, LIMA1, CARHSP1, UCP2, H2AFY, DNAJC9, AAMDC, SKA2, HNRNPA3, ECI2, PSME2, EMP3, SOX9, COMMD4, SPTSSA, RHOC, SEPT9, PSAT1, ORC6, LSM4, CNN3, CAMTA1, SYPL1, NUDT1, PFN1, DDAH1, STK17A, DECR1, CBX5, ALDH7A1, HNRNPM, VRK1, ITGB3BP, ACAA2, CKAP5, TMEM237, PMF1, HMG20B, ASRGL1, RNASEH2B, MDK, SH3BGRL, NENF, CYBA, ANXA6, PNRC2, MZT1, NFIA, EMC9, NASP, RNASEH2C, ACTL6A, SRI, SLC25A5, NUDT5, RHNO1, GGH, MSI1, PFN2, SEPT11, HDGF, PPP1CA, UQCC2, ACADM, HNRNPD, PHLDA1, PSME1, FUS, CALD1, GSTO1, HADHB, PEA15, MARCKS, SAE1, TPM4, GPM6B, GPC4, MYL6, BLOC1S1, LHX2, LRRC3B, CDK4, EXOSC8, GBAS, CD63, PPDPF, SNX5, GOLIM4, SERF2, NAA38, MPPED2, NFIC, DNMT1, ELAVL1, PAM, CXXC5, TIMM10, NT5C, PGM1, H3F3A, GLUL, HES6, DHRS7, RALY, SNRPD1, PAICS, CCDC14, ASPH, FUZ, HP1BP3, TMSB4X, CYR61, TPGS2, PIN1, RFC2, ID3, PDLIM7, BCAN, IFI27L2, PDLIM4, RPS27L, NDUFA11, VEZF1, FKBP3, HIBCH, GAPDH, JADE1, ANAPC11, BBX, ROBO1, MIDI, RPS6, SMARCA1, UBE2E3, MTHFD1, TUBG1, HIGD1A, ATRAID, HSD17B10, TRIM24, LSM14A, KCNG1, FAM96A, CXCR4, PDIA6, POU3F2, HINT2, NPC2, GMPS, CCT5, SHMT2, PFKL, SLC25A6, SEC11A, JAM2, CFL2, PDIA4, APOA1BP, HYI, PRDX6, FUBP1, MAT2A, TTYH1, BAZ1A, PGP, SUZ12, MAZ, EIF4EBP2, CORO1C, CHCHD5, RFC4, PNKD, ITGAE, UQCC3, C1orf6l, FERMT2, TMTC4, PTGES3, POLR2E, ETV1, POLR2L, FIBP, PLEKHO1, AHCY, MRPL11, TSPAN6, MYEF2, CALM3, CCBL2, PPIA, PEX2, LRRC58, TXNRD1, HLA-B, IMMP1L, MYH10, TFDP1, CTNNBIP1, HIST1H1C, HSP90B1, UQCRC1, TSEN34, NAA10, CD151, LSM3, TULP3, LSM2, CNTFR, HNRNPUL1, TMBIM4, HLA-C, COPRS, NELFE, NDUFAF3, FUT8, MPST, PRDX3, BAALC, CCNG1, SRSF6, LMAN2, ENSA, MKKS, PRADC1, SUGP2, SCRN1, FZD3, RANGRF, PPIF, FAM92A1, ADH5, RPN2, CNP, SLC35F1, PPP4C, EHBP1, HNRNPAB, MACROD1, ACYP1, SMS, ATP1B3, ZNF738, COX17, MAPK11P1L, H2AFY2, LRRFIP1, TMEM107, MINOS1, BCKDK, TUBB, MRPS16, MRPL23, ILK, MED30, SSNA1, SNX17, PTCD3, CTBP2, PSMD14, UBE2L3, RRP7A, DPM3, RPL39L, RABL6, MSI2, DGCR6L, CALR, RFXANK, GINM1, SAT1, TMA7, WDR1, SCCPDH, PA2G4, ANAPC15, STX10, C17orf89, GFAP, CHD4, MPDU1, AK2, GPAA1, UBXN4, CYB5R3, HACD3, NDUFS6, TRAM1, CCDC88A, 1P09, ACAT2, PPM1G, CTCF, TFDP2, MYL12A, NUTF2, NELFCD, HOOKS, DARS, CTDSPL2, FDX1, PCBD1, MRPS6, SPAG9, NDRG2, CENPV, GNAI2, CSTB, ARL4A, VRK3, TMEM132B, TIA1, SLC35B2, FN3KRP, CDK2AP2, SNRNP25, DNPH1, NSRP1, HRSP12, AC004556.1, UBE2E1, SLC16A1, SLF1, SUMO3, ARHGAP33, SRGAP2B, RP3-525N10.2, IFT81, GOLM1, C7orf55, ELP6, EXTL2, COMMD10, ID1, CISD2, XRCC6, ISYNA1, PDCL3, SRR, RECQL, CASP3, LPCAT1, TRIM36, STRA13, SESN3, CCND2, PSMA4, DHX15, RNF168, MIF4GD, RIT1, DNAJB1, DHX9, CNPY3, GPT2, TMEM141, REEP3, CST3, ABHD12, CTPS1, SLC39A1, APOO, KCNQ2, PIGX, SLC25A1, PEX10, CUL1, EXOSC3, TAF9B, IQCB1, JPX, NGLY1, PLOD2, CENPT, SRPK1, TUBA1C, TIMP2, PEX19, KLHDC8A, EFTUD2, SGCE, PHYH, TSC22D4, NRCAM, SNAPC1, TOR3A, SPATA33, TMEM38B, and HES4.

In some embodiments, the organoid has been cultured for about 6 months or more (e.g., about 6 months, about 9 months, about 12 months, or longer) and comprises one or more of astroglia, callosal projection neurons, cycling progenitors, immature callosal projection neurons, immature interneurons (e.g., immature inhibitory neurons), immature projection neurons, intermediate progenitor cells, outer radial glia, radial glia, and cycling interneuron precursors (e.g., cycling inhibitory interneuron precursors).

In some embodiments, the organoid has been cultured for about 6 months or more and comprises about 6%-16% astroglia. In some embodiments, the organoid has been cultured for about 6 months or more and comprises about 7%-22% callosal projection neurons. In some embodiments, the organoid has been cultured for about 6 months or more and comprises about 5%-8% cycling progenitors. In some embodiments, the organoid has been cultured for about 6 months or more and comprises about 10%-31% immature interneurons. In some embodiments, the organoid has been cultured for about 6 months or more and comprises about 2%-10% immature projection neurons. In some embodiments, the organoid has been cultured for about 6 months or more and comprises about 1%-7% intermediate progenitor cells. In some embodiments, the organoid has been cultured for about 6 months or more and comprises about 22%-39% radial glia. In some embodiments, the organoid has been cultured for about 6 months or more and comprises about 4%-8% ventral precursors. In some embodiments, the organoid has been cultured for about 6 months or more and comprises substantially no corticofugal projection neurons or immature corticofugal projection neurons.

In some embodiments, the organoid has been cultured for about 6 months or more and comprises about 6%-16% astroglia, about 7%-22% callosal projection neurons, about 5%-8% cycling progenitors, about 10%-31% immature interneurons, about 2%-10% immature projection neurons, about 1%-7% intermediate progenitor cells, about 22%-39% radial glia, and about 4%-8% ventral precursors. In some embodiments, the organoid has been cultured for about 9 months or more and comprises about 6%-16% astroglia, about 7%-22% callosal projection neurons, about 5%-8% cycling progenitors, about 10%-31% immature interneurons, about 2%-10% immature projection neurons, about 1%-7% intermediate progenitor cells, about 22%-39% radial glia, and about 4%-8% ventral precursors. In some embodiments, the organoid has been cultured for about 12 months or more and comprises about 6%-16% astroglia, about 7%-22% callosal projection neurons, about 5%-8% cycling progenitors, about 10%-31% immature interneurons, about 2%-10% immature projection neurons, about 1%-7% intermediate progenitor cells, about 22%-39% radial glia, and about 4%-8% ventral precursors.

In some embodiments of the organoids described herein, an immature projection neuron in an organoid cultured for about 6 months or more is characterized as an organoid cell that overexpresses, as compared to the rest of the organoid cells, at least about the first 5, 10, 20, 30, 40, 50, 75, 100, 125, or all 136 of the following genes: ARF4, DDIT4, SEC61G, EIF1, HERPUD1, PGK1, BNIP3, MORF4L2, ALDOA, IGFBP2, ILF3-AS1, ALKBH5, FAM162A, NPM1, ARF1, SERP1, EGLN3, DDX18, H1F0, ENO1, HILPDA, TMED9, KDELR2, P4HB, HSPA5, SLC3A2, KCNQ1OT1, LDHA, SRP54, TMED2, MYDGF, RPS5, ZFAS1, VIMP, CA9, PDK1, P4HA1, ADM, NRN1, SLC16A3, MIF, RNMT, DNAJB9, SRPRB, INSIG2, HSPA9, NANS, PGAM1, DCAF13, GNL3, GORASP2, BNIP3L, EPB41L4A-AS1, ENO2, ATF4, EIF2S2, TXNIP, XBP1, ZCCHC7, UFM1, WDR45B, RSL1D1, COPB2, ANKRD37, SEC13, ST13, TRIB3, CCDC107, WSB1, PRDX4, BOD1, BET1, EIF2A, DNAJC3, TMEM263, RPF2, RP11-798M19.6, SSR3, TAF1D, SUCO, COPB1, SLC39A7, SEC61A1, TPI1, SURF4, MPHOSPH10, HM13, SEC31A, GOLGA3, IGFBP5, PFKFB3, DNAJB11, GPI, MIR210HG, UAP1, SIAH2, FUT11, EPRS, GOLGA4, MTHFD2, DNAJB2, TMF1, SARS, MXI1, GARS, COPG1, NARF, TNIP1, PPIL3, TATDN1, CCDC47, RPA2, WDR54, EGLN1, PGM3, KIAA0907, ALDOC, SHMT2, AARS, MLEC, SND1, KDM3A, PRPF6, LONP1, EBLN3, EIF4EBP1, EIF2B1, RSBN1, VEGFA, SERPINH1, TET1, FAM210A, ELP2, IARS, ASNS, and RGS16.

In some embodiments of the organoids described herein, an immature callosal projection neuron in an organoid cultured for about 6 months or more is characterized as an organoid cell that overexpresses, as compared to the rest of the organoid cells, at least about the first 5, 10, 20, 30, 40, 50, 75, 100, 125, 150, 200, 250, 300, 350, 400, 450, 500, or all 547 of the following genes: PALMD, NEUROD2, BHLHE22, CLMP, CSRP2, SLA, ELAVL2, NEUROD6, CADM2, SEZ6L2, SNX7, CXADR, SNCA, RBFOX2, PPP2R2B, NSG1, CD24, EIF1B, MIAT, GRIA2, RAB3A, ATP6V1G2, ZBTB18, STMN2, SOX11, TSC22D1, NREP, CONI, GNG3, CPE, MEIS2, SRM, BEX1, THRA, CRMP1, APP, BASP1, RTN1, TMSB10, HN1, PTMA, EIF4A2, SSTR2, ZNF704, BEX2, ATAT1, POU3F3, APLP1, POU3F2, SEMA3C, DUSP1, PLXNA2, ZNF462, VAMP2, SVBP, TTC3, TERF2IP, PODXL2, PHLDA1, LMO3, CAMKV, LMO4, SHTN1, GAP43, MN1, ENC1, FOXG1, TBR1, KLC1, AP3S1, FRMD4B, FAM49B, NRP1, SNAP25, LRRC7, TBPL1, ETFB, CNOT2, TXNIP, EPHA4, CDC42EP3, NELL2, RPAIN, VCAN, HSP90AB1, CNR1, PBX1, CAMK4, AUTS2, IP6K2, IFRD1, TTC28, DOK6, PPP1R14C, SMARCD3, ZC2HC1A, DDX24, CCDC28B, SMIM15, GNAI1, MARCH6, CDK5R1, FAM126A, UBE2D1, HPCA, GABPB1-AS1, CCNG2, CELF2, TM2D3, VDAC3, MAP1LC3A, ENO2, AP1S1, SPTAN1, COX7A2L, PLPPR5, HS3ST1, LINC01102, GNAL, NR2F1, MAPT, PCSK1N, TTC9B, TSPAN5, TNRC6B, CAMLG, NDUFAF2, ITFG1, ARID5B, NUP93, MLLT3, APLP2, TCEAL7, CRYZL1, DAAM1, FAM215B, BAIAP2-AS1, HMP19, YWHAG, FAM13A, MKRN1, NPB, ZNF608, KIFSC, PFKM, RASGRP1, POLR1D, SARS, SCG3, FUT9, BEX4, C1orf216, NRXN1, CMAS, MMADHC, AKAP9, AKR1A1, RRAGA, RPL7L1, TRIM2, NHSL1, UCHL1, NME1, WHSC1L1, GRB2, HSPA8, DEAF1, PTCHD2, ZNF292, TMEM108, IGSF8, RNF24, YWHAH, MAP4, CHD3, EEF1B2, SRGAP1, STMN4, KCTD6, TMEM59L, SLF1, ANP32A, ATP5G1, PID1, SMIM8, FAM57B, SMARCA2, MEX3B, LRRTM2, NTM, BLCAP, CCDC112, DACT1, NUDT3, DDX1, PHF20, RP11-192H23.6, ST3GAL6, WDR47, GPR162, ELAVL3, GNAO1, EPB41L4A-AS1, ARMCX3, MRPL32, GNG2, CCNB1IP1, SPATS2, PWAR6, CEP170, ZEB2, NFASC, GNL3, C1orf52, TRAP1, ZHX1, TIPRL, PHF20L1, CAMK2B, SSX2IP, TULP4, LHX2, IDS, TMEM167B, CLASP2, TBCC, EPHB1, LDOC1, CELSR2, C5orf24, APBB1, STARD4-AS1, FAM107B, HK1, GPM6A, EML1, PLEKHA1, OCIAD2, FAM171B, PLPPR2, GALNT11, ANAPC5, CHGB, KNOP1, MPHOSPH8, SPINT2, ZNF148, SERGEF, TSPYL1, AMER2, HSF2, GRIA3, LY6H, MCTS1, DCTPP1, IRF2BPL, IFT20, C14orf132, NT5C3A, ORC4, PGAP1, LEO1, PEBP1, AC004158.3, CHCHD6, CCDC115, RP11-83A24.2, PTPN4, NEO1, APBA2, FSD1, KRR1, ACYP1, ZNF131, EBPL, CMSS1, CNOT4, CD200, PJA1, NIPA2, PRPSAP2, HARS, GPR85, SMAD2, SLC35E3, MAGEH1, FBXL15, PLXNA4, SBK1, CECR5, FARSB, BTBD10, MRPL44, ANKRD46, STXBP1, TACC2, RIC3, C3orf14, ARMCX1, TMEM35, RUFY2, SRSF8, POLR2B, TMED3, AMN1, KBTBD6, FKBP4, TTLL7, FMNL2, TBC1D14, CCDC136, SHOC2, ATL1, ZNF821, RAP1GDS1, ZNF91, BLOC1S6, RSBN1L, TRMT10C, LARP1, COPS3, JPH4, ASNS, CLIP1, PKIA, CES2, F2R, RAC3, SH3RF3, SBNO1, RNF165, ATP6V0A1, PRR7, ACTR1B, CEP57, ZPR1, RAMP2, ATXN7L3B, ZNF397, KIF3A, KIFAP3, SLC4A7, RIMKLB, MYT1L, NIPSNAP1, NDUFAF4, PPP3CB, FKBP1B, LMO1, NFKBIL1, SF3A3, HSDL1, NPM3, LETMD1, RIF1, NAA15, TAF1D, RP11-436D23.1, HDAC5, SRD5A1, PARP2, MRPL48, IGSF3, HINT3, MPZL1, EFNB2, YPEL1, RAP2A, ILF3-AS1, HMGXB4, DERL1, ARRB2, EPM2AIP1, TPT1-AS1, PAK1IP1, PLEKHA5, CDKN1B, CNKSR2, RPS6KA5, PTPRG, WDR33, GOPC, UBQLN2, GTF2B, ASGR1, FNBP1, LRIF1, ZC3H6, WDR82, ZNF766, RNF14, AAK1, ZFAND1, CELF3, XBP1, SERP2, ZNF770, KDM6B, THAP9-AS1, EXOC4, VPS37A, ING4, LINC00667, EIF4EBP1, COIL, SIAH2, BZW2, GARS, KMT2A, SLC35E2B, SH3BP5, CHST12, EIF3J-AS1, C2orf69, R3HDM2, NSMCE3, DIXDC1, EEF1A2, SCAMPI, SORBS1, UXS1, MCMBP, SNHG8, CHMP7, FRMD4A, VPS53, CAMK1D, RP11-1094M14.11, SLC8A1, ZNF622, CUL1, ELP2, NUDT11, MBTPS1, RFPL1S, C12orf65, FAM131A, ZNF7, PPID, ZC3H11A, NOB1, PUS7L, KAT8, CLK1, PPP1R10, MRPS2, FBXO22, PAK1, SLC35A1, ACOT7, MYCBP2, NOL11, THUMPD1, ITSN1, TMF1, FBXO44, PEX13, CBFA2T2, FAM217B, CLK3, ERAL1, RABIF, TUBGCP4, ATCAY, B4GALT3, GDAP1L1, RSBN1, KBTBD7, ARMC8, SYP, FSD1L, GADD45G, SNAP47, KLHL23, CSAD, TTF1, GNB5, CELF5, PHF1, BORCS8, SNHG15, ZMYND8, CDKN2D, GDAP1, PPP2R5B, HOOK2, ZFP90, MPHOSPH10, TCAF1, ZNF512, LIN7B, NOC2L, PGM2L1, PCGF2, OGFOD1, IGDCC3, NECAP1, G3BP2, SFSWAP, ACTL6B, FAM49A, FAM126B, NUDCD3, B4GALNT1, EXOSC5, SEZ6L, BBC3, SDAD1, ERICH1, REEP1, CASC3, MTPAP, C9orf72, YDJC, PURB, THAP3, RUNDC3A, BENDS, ARIH1, HPRT1, RP11-352M15.2, RPAP2, RIOK1, DPH7, WDR74, KLHL28, WASF1, ATP1A3, LARP6, DYRK2, INAFM1, CELF4, CCP110, ZNF652, NRBF2, NPRL2, NAT9, TMEM57, NETO2, GSK3B, GFOD2, FNIP2, PIK3R1, KCNQ1OT1, ARPP21, PLK2, and INA.

In some embodiments of the organoids described herein, a callosal projection neuron in an organoid cultured for about 6 months or more is characterized as an organoid cell that overexpresses, as compared to the rest of the organoid cells, at least about the first 5, 10, 20, 30, 40, 50, 75, 100, 125, 150, 200, 250, 300, 350, 400, 450, 500, 600, 700, 800, 900, or all 914 of the following genes: FGF12, MEF2C, LINC00643, TSPAN13, SYT4, GRIN2B, ARPP21, SYBU, MPPED1, PAK7, SH3GL3, NEFM, RBFOX1, JAKMIP1, SEMA7A, CAMKV, INA, TTC9B, PIK3R1, LINGO1, NELL2, R3HDM1, CCBE1, CAMK2B, HPCA, DUSP23, CELF4, MMD, FAM49A, CXADR, NSG1, GNAI1, HMP19, SYT1, SPINT2, SHTN1, SLA, SNCA, ENC1, STMN2, DACT1, RAB3A, CDKN2D, STMN4, BHLHE22, LY6H, SEZ6L2, LMO4, ZBTB18, RAC3, ATP6V1G2, NEUROD2, CD24, TSC22D1, YWHAH, DOK5, UCHL1, GAP43, MAP1B, CRMP1, STMN1, TUBB2A, BEX2, VAMP2, BASP1, GNG3, RTN1, MLLT11, PCSK1N, HN1, SCN3B, PTPN2, CADM2, INAFM1, BEX5, PGM2L1, ATP2B1, FABP7, SULT4A1, CADM3, SSTR2, BEX1, GPR85, SYT13, CDC42EP3, SATB2, ADCY1, RASL10A, MIAT, PCLO, TAGLN3, MYT1L, DEAF1, ATP6V0B, AKAP7, FKBP1B, YWHAG, GPM6A, PPP1R14C, APLP1, DLG2, CALM1, NEUROD6, RGS17, DAB1, SCG3, GABBR2, CDC42, TUBA1A, HOMER1, PLPPR5, BEX4, SERP2, TMOD1, DSTN, C1orf216, PAFAH1B3, OCIAD2, SYT5, ATP1B1, HBQ1, MAP1LC3A, PPP3CB, FAM49B, PLK2, KLC1, GNAZ, FJX1, EIF4A2, TBCB, GABRB3, TPM3, RBFOX2, DYNC1I1, DPF1, PRR7, RBFOX3, HS3ST1, WASF1, ACOT7, SNAP25, ATL1, CDK5R1, CHGA, CELF5, NREP, HSP90AB1, RUNDC3A, C1QTNF4, TUBB, RNF165, PEBP1, VSTM2B, AASDHPPT, SNX7, CLMP, ARPC2, GPRIN1, ETFB, YWHAZ, FAM57B, CSRP2, RP11-356J5.12, F12, DNAJB6, GDAP1L1, CLTB, TMEM59L, TUBB2B, DOK4, ATP1A3, SCN2A, CORO1A, LY6E, KIAA1107, PFDN2, PPP2R2B, GNAL, CELF3, SLC8A1, TMEM14A, SLC38A1, LRRC7, PPFIA2, LIN7B, PRKCZ, REEP1, TMEFF2, PPP3CA, PCMT1, BZW2, PODXL2, SH3BP5, ATCAY, AP1S1, L1CAM, PHACTR3, HS6ST3, STX1A, CAMK4, HIVEP2, HSPA8, ASPHD1, EPHA4, YWHAB, ARPC5, CSRNP3, ELAVL3, RNF187, EXOC4, EFNA3, TAF9, MIR124-2HG, PPP2R5B, ATAT1, DLL3, YPEL3, LDLRAD4, CALM3, PLPPR2, SRM, MAPT, TXNIP, GRB2, NPB, COTL1, BCL7A, RAB33A, NUDT3, NRXN1, DRAP1, MYCN, GFOD1, THY1, NTRK3, CHGB, RFPL1S, ACTL6B, TCEAL2, ADD2, NDUFA5, PTPRO, ANKRD46, TM2D3, C6orf1, ANKRD12, CSNK1A1, AFF3, RAMP2, ATP5G1, CHD5, ARG2, TMEM160, DAAM1, NAP1L3, NUDT11, SMAP2, POU3F1, STXBP1, RNF182, DISP2, KIF3C, PRKAR2B, LINC00599, CDK5, SSX2IP, STOML1, OLFM1, BLCAP, RFK, PNMA1, CMAS, NDRG1, MAPK8, RAP1GDS1, TSPYL1, HCFC1R1, TULP4, UBE2V2, PSD, DDX24, SLC25A4, SERINC1, NECAP1, PLPPR1, SYP, CTXN1, TNNT1, COMTD1, FAXC, ILF3-AS1, GTF3A, FBXL15, MARCH4, AUTS2, MPP6, FLRT2, NME1, CYCS, ENO2, PTPRD, NDUFAF2, PRKACB, CA11, MAPRE3, EML1, RUNX1T1, VPS29, CD200, NAPB, NUDCD3, ANK3, ACTR3B, ST6GAL2, NMNAT2, CHCHD6, AP3S1, ARID4A, TCTEX1D2, ZBTB38, ST3GAL6, CCDC112, SRD5A1, CDKL5, CELF2, GRAMD1A, SOBP, GFOD2, HSDL1, KIF3A, NUDT14, TMOD2, AGTPBP1, DIRAS1, TTC9, GABRB2, H1FX, TUBB4A, KIF5A, ATOX1, TMEM35, CACNA2D1, C10orf35, TMEM150C, THRA, FGF13, BID, CDKN2AIPNL, APBB1, WAC-AS1, C5orf24, C2orf69, RIC3, C9orf16, SBK1, FNDC4, SRRM4, TTLL7, SLBP, MKRN1, YDJC, IDS, ZC3H15, AKT3, KLHL8, MORF4L2, NEO1, SNAP91, ZEB2, TCEB1, DPYSL5, FSD1L, EIF4EBP1, NDN, STRBP, PARP6, RASSF2, KIF5C, FSD1, C12orf76, MPC2, PARD6A, RGS7, FAM134A, ST3GAL1, ATP6V1B2, NBEA, RPS6KL1, GNB5, TMEM57, KCTD13, NDEL1, PPFIA3, PAK1, DEF8, GNAO1, ASXL3, CAMLG, RELL2, MEAF6, CAMK1, KIF3B, HK1, COX7A2L, HIVEP3, SPTBN1, CACNB3, JPH4, ELOVL4, CCDC184, TBC1D14, MEX3B, CDH11, TIPRL, KIDINS220, BAIAP2-AS1, BTBD10, DTX3, TMEM151B, TMEM108, TCEAL7, DCAF6, MYCBP2, KIAA0895L, SLC12A5, GABPB1-AS1, ANKS1B, CPE, PEX13, FNIP2, FAM126B, PTBP2, NOL4, PLXNA4, HDAC5, DLGAP1, POP7, RNF11, PPP3R1, CELF1, LHX2, BORCS8, KBTBD6, PLPPR4, SCAMPI, KLC2, KIFC2, AMD1, MAST1, DCTN3, KIFAP3, SEC11C, ZNF821, PPID, FARSB, RP11-127B20.2, GOT2, NCOA1, NTM, FAM126A, ARL10, HSD11B1L, RAB2A, CNR1, GRIA1, ANK2, RABEP1, GNL3, SV2A, MAP4, PPP2R1A, MRPL18, VTI1B, RUFY3, SCAMPS, GNB1, RP11-352M15.2, ACYP1, PHAX, YPEL1, WDR47, ATP13A2, ROGDI, GNG2, PHACTR1, CCDC90B, HINTS, C17orf58, USP11, RAPGEF2, BBC3, IGSF3, SEPT6, AFAP1, PITHD1, GIT1, PRDM2, FRMD4B, SMIM8, FAM117B, CRK, FAM188A, SLC35E3, TSPAN14, ODF2L, SLC44A5, PKIA, FAM155A, DDX25, RIMKLB, GPR162, RP11-382A20.3, SBNO1, ATP6V0D1, MAP6, CLASP2, EPHA5, MPZL1, ARHGEF7, KLHL23, PDIK1L, PCDH7, ZMAT2, MAPRE2, RNF219, C16orf45, TNRC6B, ARF3, FAT3, CMIP, SPOCK1, AK1, RRAGA, ZNF302, NRXN2, CDK19, CAMKK2, KIF2A, ATXN7L3B, ITFG1, LINC00657, DYRK2, C9orf78, ARHGAP33, PBX1, PAIP1, AMN1, TRIM3, RUSC1, CCSAP, MICAL3, PJA1, TMEM178B, SSBP4, PRKAR1B, ATXN10, MSRA, SHOC2, SPIN1, PSMG4, PTP4A1, ZBTB44, ZNF148, ZWILCH, DTNBP1, PNMA2, OPTN, DTD1, FRMD3, B4GALT5, MAP7D1, CEP126, DUSP8, MYO5A, ZNF622, CACNG8, NAP1L5, SPTAN1, TSPO, ST8SIA2, MAGEF1, TRAPPC4, TBRG1, SESTD1, UBQLN1, FAM131A, TCAF1, SLC16A14, LINC00632, RABEPK, UBL4A, ARMC1, SERINC3, ITSN1, FAM89B, ZC3H6, PLPPR3, MRPL44, ATP9A, SORBS2, VPS4A, CDC37L1, PAK1IP1, LDOC1, DYNC1LI1, HOOK2, RAB14, RNF113A, ASNS, SNHG15, ZNF793, TNFRSF21, PAFAH1B2, TOMM70A, RIMKLA, KALRN, MCUR1, ENDOG, C1orf52, RNF146, RP11-83A24.2, TMOD3, TCP1, C12orf73, PPP1R9A, CTTNBP2NL, ZNF74, DYNLRB1, IRF2BPL, GALNT11, ALAS1, CCP110, CNIH2, SMARCD3, LINC00667, LSM10, CCDC136, SS18L2, RNF145, TSPYL4, NT5C3B, SRPK2, CACYBP, B4GALNT1, KATNB1, BRSK1, RABL2B, AGAP3, FAM217B, MIR181A1HG, BOP1, IGSF8, FARP1, AHSA1, SH2B2, PDZD4, FKBP4, PAFAH1B1, HARS, PCGF3, PRR3, NETO2, LONRF2, HEBP2, DIXDC1, ENTPD6, SCAI, RALA, PRKAR1A, AAK1, RNASEH1, PIP4K2B, TRAPPC6B, ZNF281, ATP2C1, TRIM2, CLIP1, KIAA1549, SEPT3, PSD3, ZNF566, GPR161, RP11-192H23.6, DUSP6, EXOSC6, MAPKAPK5-AS1, LTBP4, NIPSNAP3A, COPS7B, GOPC, COMMD9, STMN3, ELOVL6, STOX2, G3BP2, ACVR1B, ADGRL1, SRCIN1, BDP1, GRIA3, PKN1, PARP2, TIMM17B, SEC14L1, RBM15B, ERC1, CSNK1E, EPM2AIP1, MED13L, TMEM167B, PIANP, ATP1A1, TRUB1, MORN4, HMOX2, KNOP1, THAP9-AS1, PCSK7, KIAA2022, MAP9, ZYG11B, SGSM3, IFT20, MED19, NOLC1, AMER2, FOXJ3, AC004158.3, LETM1, MAP1A, GPRASP2, ATP6V1A, KMT2A, MAX, DPYSL3, EGLN1, GRIN2A, GNAQ, BAG4, BOLA3-AS1, LIN7C, PTDSS1, MAP1S, EPN1, TAF6, UBALD1, SLC25A17, ATP6V0A1, WDR82, MRFAP1L1, UBE2O, SLC25A36, XXYLT1, SH3RF3, DNAJC12, RPAP2, NFASC, CHORDC1, UGCG, ZNF652, TSSC1, UCHL5, PPP1R2, IFNGR2, UCHL3, SFXN3, BLOC1S6, CHRNB1, FEM1B, SPPL3, DNM1L, CAMK2G, TRAPPC2, ZMYND8, FAM228B, TMEM192, SNAP47, INPP4A, PTPN1, CHN1, RHOB, FAM177A1, UBQLN2, IRGQ, NOVA2, LRRC49, EIF2AK4, BRD2, RPUSD3, C15orf57, NR2C2AP, CCDC107, HERC1, CAMK1D, EXOSC5, MTMR4, CAMSAP1, UBE2Z, RALGDS, ZFAND2A, CCDC186, FBXO45, GPATCH2L, DCAF10, NAV3, C1orf2l, ARRB2, TSPAN5, DDX51, TSPAN7, OGFOD1, ZMYND11, DUSP12, FEM1A, HSPH1, CENPT, SEH1L, NAA15, LRP12, GAREM1, LRRC40, ZC3H8, ZNRF1, ZNF445, ISYNA1, MTMR9, RALGAPA1, AKAP11, KIAA0930, ZBTB37, CAMSAP2, C11orf95, CSRNP2, SLC35B4, TRAP1, RAB6A, PPP1R18, JARID2, C16orf72, ANKRD13D, GGT7, HCG18, GMEB1, R3HCC1, SLC22A17, GABPB1, PDHA1, GFPT1, CC2D1A, LARP1, DCTN5, BMPR2, DCTN1, AKIP1, CCZ1, DCAF7, ZNF32, RP11-660L16.2, LRP3, MLXIP, ATP6V1H, NUTM2B-AS1, MSL1, ATAD1, CAND1, CAP2, ABL2, VPS53, MTURN, CLIP3, TRIO, R3HDM2, ZFAND2B, SECISBP2L, FAM219B, ASGR1, SMARCA2, PPIL2, DERL1, ABR, ADGRB3, NCOA6, JOSD1, ABHD6, ARHGAP35, PRCC, EIF2B2, MYO9A, CUL2, FAM98A, USP7, BAIAP2, HECTD4, ANKRD36C, WDR20, MLX, SAMD14, SIGMAR1, FHOD3, NAV1, ISG20L2, POGK, PDXDC1, SBF2, YY1, ARHGEF12, ZNF639, SHISA5, ARHGEF9, ATMIN, GATAD2B, EXOSC10, ZNF512, and PANK3.

In some embodiments of the organoids described herein, an intermediate progenitor cell in an organoid cultured for about 6 months or more is characterized as an organoid cell that overexpresses, as compared to the rest of the organoid cells, at least about the first 5, 10, 20, 30, 40, 50, 75, 100, 125, 150, 200, or all 235 of the following genes: EOMES, CPE, TMEM158, CLMP, MLLT3, RASGRP1, SMARCD3, SEZ6L, SERPINF1, UNC5D, MARCKSL1, CXCL12, PPP2R2B, CNR1, GNG3, CYB5A, CNTNAP2, SOX4, TBR1, CDC42EP3, MEIS2, CORO1C, GPM6A, SSTR2, LYPD1, GAP43, DOK6, TFAP2C, CSRP2, MLLT11, UBE2E3, EEF2, IER2, RPAIN, TMEM108, ASCL1, ZFHX4, MAPRE1, EPHA3, CALD1, MN1, POU3F3, ZBTB20, FMNL2, MIR99AHG, EBPL, PGRMC2, KIAA1715, POU3F2, MYO6, RP11-436D23.1, NR2F1, IGSF10, RP11-553L6.5, MAGED2, FRMD4A, SCARB2, PTPRS, TMSB10, MTCL1, ATP1B3, DAAM1, SYNE2, UBE2D1, FIGN, LSAMP, PBX1, CMC1, DDAH2, RBPJ, LUC7L3, LRP8, SEZ6, SRGAP3, EPHB1, GPRC5B, CIRBP, STMN4, DLL3, C12orf49, CCNG2, ZHX1, FUT9, BLCAP, PHYHIPL, RAB3A, MAP2, BTG2, GULP1, BBX, TERF2IP, OSBPL6, GADD45G, MEX3B, TRIM2, FAM126A, BAZ2B, GTF2I, SETD7, INSM1, EML1, ABRACL, ZC2HC1A, ARID5B, BICD1, GRIA3, ATP6V1G2, C1orf54, TFDP2, MPPED2, PRMT1, RPN2, NUP93, EMX2, VCAN, SRGAP1, WIPF3, HEBP2, IGFBP2, FZD3, TIMP3, MDK, PCBP4, NFKBIA, MLLT4, SCRN1, MLLT4-AS1, ELAVL2, FGF13, DLEU1, SPIRE1, KNOP1, C14orf132, MIDN, ATAT1, LPCAT1, NFIX, AIP, BEX1, TCAF1, TANK, KDM5B, CYTH1, MDFI, ITGB1, HDAC9, DTD1, APLP1, EVL, GSTA4, HDAC5, TP53RK, SEMA6A, MBTPS1, BMPR1A, PJA1, ARL4C, ZMIZ1, LDOC1, LHX2, PCMTD2, SPATS2, CDK5, CPNE1, LTA4H, ELMO1, NARF, INTS6, TNRC6B, IP6K2, IVNS1ABP, ZBTB18, ZKSCAN1, RPA1, RP5-1085F17.3, GPC2, RP11-76114.1, NTM, POU2F1, TBPL1, HERC2, FNBP1, CALCOCO1, PLPPR1, BEX2, LINC01102, SOBP, CXXC5, NIPSNAP1, HPCAL1, SENP6, RBFOX2, KDM6B, STARD4-AS1, QSER1, NF2, CAMK2G, C15orf61, ING4, AC013461.1, BCAR1, MEX3A, APBA2, CBFA2T2, IFI44, SLC39A10, HSDL1, LIN7B, GRAMD1A, SMIM8, USP3, PLEKHA1, TBCC, R3HDM2, ATP6V0A1, AUTS2, RAB8B, IRF2BPL, GDAP1L1, TMTC2, FOXN2, SYP, USP46, FAM217B, KLF3, CPT1C, AC004158.3, HSD17B11, ADNP, CCSAP, PCDHB2, UBALD1, SOGA1, SBK1, and FAM60A.

In some embodiments of the organoids described herein, an immature interneuron in an organoid cultured for about 6 months or more is characterized as an organoid cell that overexpresses, as compared to the rest of the organoid cells, at least about the first 5, 10, 20, 30, 40, 50, 75, 100, 125, 150, or all 155 of the following genes: DLX6-AS1, DLX5, SP9, PLS3, ARL4D, GAD2, TAC3, DLX1, DLX2, MEST, ARX, RASD1, ELAVL4, RND3, TMEM123, CCDC109B, DCX, PFN2, TCF4, SOX4, TMEM161B-AS1, ENAH, TMSB10, HMGN2, ACTG1, HNRNPK, DDX5, TUBA1A, ACTB, H3F3A, SH3BGRL3, RPS11, DCLK2, DPYSL3, DYNC1I2, SLC25A6, AES, ST18, HNRNPA1, DBN1, SMARCB1, HDAC2, CADM1, OLA1, PAIP2, PFDN4, DLX6, ARL4C, FXYD6, TRIM13, CCDC88A, TMSB15A, UBE2I, MSI2, NME6, H2AFY2, MAP2, CITED2, RBBP4, GAD1, KLHDC8A, SMARCA4, ROBO2, CRIP2, NFIA, PRKX, BCL11A, CHD7, SUB1, HTATSF1, TSC22D2, FSCN1, DST, SMARCE1, PAK2, CENPV, PTS, TOX3, PNRC1, BCL11B, MGEA5, NAP1L4, DLGAP4, SRSF6, CBX1, KCNQ2, ARL6IP6, FAM89B, RPA1, CHD4, RNASEH2B, POU2F1, CORO1C, SMARCD1, KLF7, MLLT4, KAT6B, PHF14, ATP2B4, LRRN3, FOXO3, ANAPC15, TDG, SERINC5, CREB1, PAK3, GPC2, PEG10, FAM210B, CERS6, SPATS2, XRN2, ASAP1, INSM1, RBP1, TIA1, LRRC40, SECISBP2, ACIN1, GSE1, CHD3, SP8, BAZ1A, FOXN3, CELF1, CASC15, MEX3A, SMARCC1, CDCA7, RAB8B, SP3, RARS2, MAGI1, LIMD2, VEZF1, GADD45G, CCDC112, DPYSL4, TCF12, PLK2, ERV3-1, HMGB3, USP3, MED17, RBM4B, CMIP, ZNF3, RAB3IP, PHACTR4, SMOC1, TIAM2, FAM60A, SEZ6, GLCCI1, and LINC01315.

In some embodiments of the organoids described herein, a ventral precursor in an organoid cultured for about 6 months or more is characterized as an organoid cell that overexpresses, as compared to the rest of the organoid cells, at least about the first 5, 10, 20, 30, 40, 50, 75, 100, 125, 150, 175, 200, 250, 300, 350, 400, 450, 500, 550, 600, or all 605 of the following genes: DLGAP5, ASPM, UBE2C, CCNB2, TROAP, FAM64A, TTK, NUF2, CDCA3, CENPF, MKI67, GTSE1, CDCA8, KIF23, KIF2C, PTTG1, TPX2, CDKN3, CCNA2, NUSAP1, BRCS, KIF4A, SGOL2, TOP2A, AURKB, PBK, HJURP, PRC1, TACC3, CASC5, SGOL1, ECT2, CKAP2L, KIF11, NDC80, MXD3, CDK1, ARHGAP11A, DEPDC1B, HMGB2, CRNDE, KIFC1, CKS2, KNSTRN, KPNA2, SPC25, RACGAP1, MIS18BP1, CKAP2, MAD2L1, CDC25B, KIF20B, SMC4, UBE2S, CENPW, CENPN, TUBB6, KIF22, TUBB4B, UBE2T, CDKN2C, CKS1B, H2AFX, ZWINT, HMGB3, MZT1, SMC2, LMNB1, TMPO, TUBA1B, BUB3, H2AFZ, H2AFV, RAD21, ANP32E, HMGN2, LSM5, HMGB1, NUCKS1, HNRNPA2B1, RAN, YBX1, RBMX, DCXR, BUB1B, DTYMK, SPC24, NCAPG, CENPU, RTKN2, EMC9, SFPQ, 01P5, SPAG5, DBF4, KIF15, TYMS, GPSM2, FOXM1, KIAA0101, MELK, MND1, FBXO5, DDX39A, HIST1H4C, EZH2, PSRC1, ILF2, LBR, CENPK, POC1A, RRM2, SKA3, STMN1, TMSB15A, HNRNPM, PARPBP, SRSF3, CENPM, SHCBP1, RAD51AP1, SKA2, HMGN1, KIAA1524, DEK, H3F3A, MARCKS, CCDC34, NCAPH, HNRNPA3, SPDL1, TUBB, CENPH, C21orf58, ESCO2, DLEU2, SKA1, RHNO1, NCAPD2, HNRNPR, VRK1, KMT5A, CMC2, ASRGL1, HES6, PSIP1, ERH, CDCA5, LRR1, GPX4, TRIP13, PLK4, CLIC1, EEF1D, ASCL1, HNRNPH3, LSM4, NUDT1, GGH, CCT5, NCAPG2, DAZAP1, PSMA4, ANP32B, USP1, FBLN1, UCP2, NPY, PIN1, CKB, FANCI, ASF1B, RCC1, CBX1, SAE1, PTMA, CDCA4, CEP57L1, DIAPH3, FUS, RAB13, C12orf75, BARD1, EXOSC8, MIS18A, HNRNPA0, GMNN, PCM1, RFC3, HNRNPDL, FUBP1, TPM4, ANLN, LMNB2, DLX1, SNRPG, ACTB, CENPC, SEPHS1, CDK5RAP2, HNRNPU, CHEK2, ORC6, SEPT10, CEP135, BTG3, SNRPB, PHF19, RBM8A, COQ2, PSME2, RANBP1, ACTL6A, DLL1, ZEB1, CENPJ, GSX2, RAB3IP, TXNDC12, TAC3, DESI2, TRA2B, IKBIP, RNASEH2A, GAD2, PIM1, CBX3, JADE1, FANCD2, DCP2, BANF1, PDGFRA, DLX2, GNG4, SMC3, BRCA2, UHRF1, EIF5A, H2AFY, TK1, CHIC2, RPA3, BCHE, NAP1L1, MAD2L2, HNRNPUL1, ZWILCH, HAUS8, CHTF18, SMC1A, SUGP2, RNASEH2B, WEE1, PFN2, PTGES3, TMEM237, SAC3D1, RDX, SRSF7, SRSF2, PKMYT1, NUP35, PPIA, RPL39L, CDK6, ATAD5, CEP97, USP13, DCLRE1C, TPRKB, SYNE2, LCORL, CBX5, DNAJC9, INSM1, RRM1, LINC01224, ANAPC15, CCDC167, NEDD1, TIMM10, SNRPD1, CORO1C, MAGOHB, TUBG1, HNRNPH1, C4orf46, PHGDH, NCAPD3, RALY, NUP37, MPHOSPH9, TFDP2, PCBP2, RHOBTB3, GAS1, ANAPC11, QSER1, ATAD2, ACYP1, C18orf54, ITGB3BP, G3BP1, NONO, GINS1, WDR34, SEPT11, MPST, CSE1L, CCDC109B, CEP152, HDAC2, MAZ, TMEM106C, TCF12, PRADC1, MAGI1, TEX30, TPR, SYNCRIP, ILF3, PSMC3, GMPS, SRSF1, LSM8, PHIP, WHSC1, SSRP1, LSM14A, FANCG, SIVA1, ODC1, CEP131, ITGAE, XRCC6, IDH2, PRIM1, CKLF, ELAVL1, MED30, EGFR, MCMI, SMS, IFI16, PGP, CTCF, SNRPC, BAZ1A, ITGB1BP1, CHD7, TIMELESS, TLE1, ARHGAP33, CBX2, NT5DC2, BRCA1, BCL7C, ENY2, RFWD3, HAT1, PTBP1, HNRNPAB, SNRNP40, SRSF10, TOX3, POLA2, UPF3B, NASP, NUP107, PMF1, RFC5, CCDC14, HNRNPD, SUV39H2, SET, NUP62, CNTLN, NUP50, MYH10, MYBL2, HAUS6, XRCC5, PFN1, FBL, NSMCE4A, SERINC5, LSM3, CPSF6, SNRPD3, FUZ, DKC1, NELFE, DSN1, KDELR2, SNRPA, HN1L, ALG8, CENPQ, FKBP3, HIRIP3, HAUS1, SMARCC1, CACYBP, FAM60A, CAMTA1, VBP1, XPO1, SRPK1, COMMD4, PSMB3, HMGXB4, CA14, ZNF738, TMX1, TUBA1C, FAM136A, RBBP7, CBFB, PPIH, CBR3, LSM6, NFYB, CTDSPL2, MAT2B, CEP57, TULP3, KPNB1, UQCRC1, LUC7L2, GNB4, KATNA1, GLCCI1, UQCC2, TIA1, FEN1, RAB8A, NFATC3, SLBP, TBCD, MAGOH, ANP32A, PAICS, MTFR1, AAAS, TARDBP, H2AFY2, PLXNC1, CTNNBL1, GEMIN2, C16orf87, CPSF3, DCTPP1, TEAD2, HSD17B10, UFD1L, SRRT, NME4, THRAP3, NUDT5, SP8, IGF2BP3, CEP78, CSTF1, FAM76B, CHCHD3, EHBP1, ING3, PA2G4, PPP1CA, OLA1, POLR2D, TMEM97, CDT1, CHAF1A, ZNF714, ARFGAP3, MRE11A, POLR2E, SKP2, HNRNPL, DHX9, NSRP1, SF1, STAG1, CTNNBIP1, SRGAP2B, TOPORS, CDK2, VEZF1, MFGE8, EIF4EBP1, PIP4K2A, DHFR, NKAIN3, TMEM18, MCM4, FAM104B, CASP6, C19orf48, DCPS, TRIM24, ZBED1, SCAF11, LRRCC1, GMCL1, RCC2, GINS2, HADH, DSEL, SUMO3, THAP9-AS1, PRKDC, ZNF680, RNF168, TOPBP1, TP53I13, PKNOX1, NUDT21, RBM14, ZNF273, CHRAC1, MMS22L, CCP110, RSRC1, SLC36A4, PPP2R3C, PSMB9, NCAPH2, RRP7A, INIP, CRB1, STT3B, CDCA7L, CTPS1, CEP89, ING5, EXOSC9, ALDH9A1, EMP2, TCERG1, NUP54, BAZ1B, PPIL1, DHX15, PDS5B, RBM25, BRD7, LARP1B, ECI1, CERS6, ASAP2, EIF1AY, ANKRD10, DNAJC2, ILKAP, RNASEH2C, NIPA2, CHEK1, SMAD9, CCBL2, TNPO3, TFDP1, USP39, KAT7, PAQR4, NUP88, LYAR, TWSG1, CLIC4, ACTN4, AGO2, PRR34-AS1, DHTKD1, NUP155, SPCS3, ASH2L, SF3B3, CDYL, AHCY, MLH1, DHRSX, CMTM6, SAAL1, U2AF2, UBR7, MCRS1, ATG4D, PHTF2, NUP58, PPM1D, PSMG1, MOB1A, SMC5, CHD1, ZNF92, MEST, MRPL23, SMC6, THOP1, ARL13B, ZFP91, KHSRP, C4orf27, MBD4, and MACROD1.

In some embodiments of the organoids described herein, an astroglia in an organoid cultured for about 6 months or more is characterized as an organoid cell that overexpresses, as compared to the rest of the organoid cells, at least about the first 5, 10, 20, 30, 40, 50, 75, 100, 125, 150, 175, 200, 250, 300, 350, 400, 450, 500, 550, 600, 650, 700, 750, 800, 850, or all 893 of the following genes: NTRK2, TPPP3, GJA1, S100A10, AGT, PIFO, ANOS1, GRAMD3, IGFBP7, NMB, CRB2, RARRES3, CRISPLD1, BBOX1, OGFRL1, CD44, CTSH, C1orf194, ITGA6, GADD45B, DCLK1, GFAP, ITM2C, CLU, AQP4, FIBIN, PLP1, HES1, IGFBP5, HEPACAM, KCNN3, B3GAT2, PAQR6, HEPN1, FAM107A, RGMA, TSC22D4, PRDX6, CCDC80, CEBPD, APOE, ZFP36, CD99, ADDS, PLTP, LAMP2, BAALC, EMP3, CDO1, ANXA1, CA2, DTNA, FSTL1, PLA2G16, F3, METRN, ZFP36L1, TSPAN3, PSAT1, SCRG1, CD9, CD81, MLC1, DDAH1, B2M, TTYH1, AP1S2, ENHO, GPR137B, TIMP2, GPM6B, PHGDH, ATP1B2, QKI, PMP22, S100B, ID4, NPC2, CRYAB, BCAN, AK1, SPAG16, NDRG2, VIM, PON2, DNER, NLRP1, HLA-C, CNN3, SOX9, SH3BGRL, MT-ND2, GABARAPL2, MT-ND3, MT-ND1, PMP2, PRDX1, EZR, TNC, ITM2B, SEPW1, MT-001, PSAP, MTRNR2L1, PEA15, CST3, FOS, MT-ND4, MT-CYB, PTPRZ1, GCSH, DBI, LGALS3, MT1F, ANXA5, SSPN, ERBB2IP, CTNNA2, NEAT1, AC015936.3, MT-ATP6, AHNAK, C5orf49, RHOC, CSPG5, RHOA, SNX3, RAB31, TIMP3, HLA-A, HIGD1A, ALDOC, SOX2, SLC1A3, TPST1, MPC1, ACYP2, VCAM1, LAMP1, CD38, PSRC1, TRIM47, TMEM47, GALNT15, SPTBN1, DKK3, HSPE1, ZFP36L2, PTGDS, JUNB, C1orf122, TMED10, OAT, CHPT1, CETN2, MGST1, ATP1A2, GLIS3, CIB1, FBXO32, CTNND2, S100A16, LYRM5, IQGAP2, RNF19A, PLEKHB1, CNRIP1, ADCYAP1R1, UG0898H09, FEZ1, GDPD2, CSTB, FAM198B, AHCYL1, GLIPR2, DDR1, MT-CO2, PAM, DST, ALDH2, CD59, TAGLN2, SERPINB6, ARHGAP5, MORN2, DNPH1, TM7SF2, LINC00998, KLF6, SOD2, GNPTAB, CD63, APC, GPRC5B, FAM181A, COPRS, ZFYVE21, ADGRG1, ANXA6, NFIA, SEMA5A, TMEM9B, ACO2, MGAT4C, PLPP1, MLF1, DCLK2, SFT2D1, SCD, SPARC, SCD5, FERMT2, WLS, OSGIN2, HADHB, ID3, ALDH7A1, PTTG11P, EPB41L3, ARRDC4, CBR1, PBXIP1, TIMP1, BLVRB, HSD17B12, DPP7, SDC3, CAMK2G, FHL1, CRYL1, POLE4, LPAR4, RHEB, PHLDA3, BDH2, ELOVL5, LGALS3BP, MTRNR2L12, LAMTOR4, LIFR, PPP2CB, GNAI2, PFKFB3, PDLIM4, SELENBP1, HLA-E, SORL1, PLPP3, FEZ2, SCN1A, CFI, UBE2E1, COMT, LRRC17, ARL6IP5, ADGRV1, PDLIM2, TMEM255A, KIF9, LRRC3B, ATP6V0E1, CTNNA1, ASAH1, CANX, PRCP, RFX4, MTRNR2L10, UBL3, TMBIM6, ZNF385A, NKAIN3, DOCK7, SEPT2, GBAS, DAAM2, GNG12, TNFRSF1A, PTRF, SQSTM1, PPP1R1C, FAM181B, JAM2, SDHC, ACTN1, SLC7A11, MOXD1, SPTSSA, REEP5, ID2, PDCD6, MAPK8IP1, KLHDC9, TMEM132A, TRIM9, DHRS4L2, AIG1, HINT2, EFEMP2, IL33, C1GALT1, PSME1, PSENEN, NPDC1, PPT1, LRRCC1, FKBP2, SYPL1, CASC4, NFE2L2, NAMPT, CHPF, ABCA1, C1orf54, ADK, SCARA3, SCP2, RAB5A, PTPRA, NDFIP1, LINC00844, EDNRB, ASPH, DAD1, FADS2, SPECC1, EFHD1, MAPK1, MAN1C1, RAB7A, CXXC5, PLEC, PTCHD1, FAM213A, ACAA1, PDPN, UBE2H, ST5, YBX3, NADK2, GAS2L1, DECR1, TP53I3, IRS2, NCAN, PLCD3, MID1IP1, PRUNE2, IFI44L, EPDR1, NUDT4, NDP, EMC2, NDUFB5, ACAA2, HACD3, ADAMS, LRIG1, CYR61, VAMP3, LRP1, TMEM163, DAG1, MLLT1, SIRT2, NME3, SDCBP, RNF13, CTSL, DHRS3, SLC25A18, SBDS, PEPD, SESN3, CH17-189H20.1, GTF2F2, PSME2, TNIK, DPY19L1, STON2, SOX21, SEPT8, PLSCR1, TP53TG1, CDC42EP4, MT-ND4L, PRNP, ELN, ACADVL, SLC25A5, SNX5, LTBP3, PCDH9, B4GAT1, DAZAP2, LIX1, NES, SLC9A3R1, LAMB2, TMEM134, CHCHD5, IGDCC4, MYO10, ENKUR, IGFBP4, OBSL1, PHYHIPL, PPM1K, SEC11A, VMA21, ROM1, AR, CRIPT, NPAS3, APC2, GNA13, RAP1A, NAV1, RCN1, LRP10, SPCS1, ITPR2, EFHC1, PKIG, DDX3X, SEC22C, ANXA7, RP11-620J15.3, C2orf72, RHOQ, PRPS1, ITGB8, SH3BP2, MAP3K5, PPFIA1, PLXNB1, TMEM205, ARNT2, LRPAP1, PITPNC1, MSRB2, BCKDHB, CARD19, FLNA, HRSP12, ITPKB, SLC16A9, MRPS14, TAPBP, IQCK, SDCCAG8, TKT, MAPKAPK3, NINJ1, PPIC, MARVELD1, WASF2, TRIP6, GRN, DENND5A, GLUD1, HMGCS1, GNPTG, PDLIM3, NSMF, PPA2, UROD, NRBP2, IFT22, SAP30BP, ABAT, GAB1, MSN, MIF4GD, AKR7A2, ATF3, TIMMDC1, IL6ST, SYNM, C16orf74, RFTN2, OSBPL11, CTSB, STAT3, PSMB8, MOCS2, FAM171A1, WDR1, TCTN1, SLCO1C1, FGFR3, C1QL1, GALK1, PSMB9, ARHGAP12, ITGA7, SNCAIP, TMEM179B, WWC1, MRPS28, APOA1BP, HIBCH, DNALI1, GYG1, CREM, PALLD, FAM134B, CTD-233602.1, GAN, CD151, STXBP3, SEPTI, HSD17B8, CNP, MPV17, GSTK1, TMED7, TRAPPC6A, ACOT13, SAR1B, RHBDD2, PHYH, ZDHHC2, CPNE2, NNT-AS1, ARL8B, RAB9A, MRC2, CCNL1, AXL, IFT43, NIPSNAP3A, BCAP31, FIGN, HIPK2, MRPS6, PIR, RPL22L1, AP006222.2, CHCHD10, FMN2, LRTOMT, MSMO1, ARHGEF10L, AKTIP, SMOX, SORBS1, SPON1, SSFA2, RIT1, LYPLAL1, KLHL5, LHFP, OXA1L, G6PC3, NACC2, SAMD8, PRSS23, CBY1, TRPS1, EVI5, SFXN5, RSU1, CYHR1, SLC25A26, CAPN2, SALL2, DHRS4-AS1, RBM38, CCS, CH17-340M24.3, MARCH2, MTSS1L, TMEM107, PRAF2, PEX2, RMDN3, PDXK, RASSF4, YAP1, CASK, FAM69C, ALG14, CPEB2, SLC6A8, ROBO3, SH3GLB1, MBNL2, PSPH, SPRY2, TMEM170A, TAB2, CD58, PCBD1, NECAP2, TSPAN6, RHPN1, C11orf49, ERBB2, DPCD, PRTFDC1, UBXN11, CTSF, EMID1, LINC00116, HSDL2, VCL, LAP3, STARD7, IMPA1, RP11-263K19.4, LPP, BMPR1B, GPR37L1, ASTN1, FMNL2, P4HTM, BBS2, SMAD1, AP2B1, SPG20, NEK6, SLC40A1, DYNC2LI1, FBXO30, ARL8A, EEPD1, YIF1B, MAGT1, TWF1, HSD17B4, WASL, ATP6V1C1, NKAIN4, KCNJ10, NPEPPS, MFHAS1, IFT57, RP3-325F22.5, CDS2, PTPRF, HHLA3, MYL5, FAM199X, SPATA20, SEPN1, TPP1, TTYH2, NMD3, FAT1, COL6A1, SUCLG2, MPDZ, LMBRD1, C5orf56, FOXK1, CAST, HOMERS, RAB29, PAQR8, CTSD, CMBL, AMFR, RNF141, ABCD3, RAB21, HS6ST1, TMED5, RENBP, TMED1, MEGF8, TOM1L2, HMGN5, FBXO8, HEATR5A, RGL2, C2orf76, ARAP2, SWI5, NT5C, LTBP1, ACBD5, SEMA6A, NAV2, S1PR1, SLC12A4, HSCB, PTRH1, FAM174A, B9D1, EFCAB14, VEPH1, TJP1, ZDHHC12, TMEM50B, TFPI, CYB5D2, LIPA, BMP7, AGTRAP, CDC42EP1, IVD, AGGF1, ANAPC10, HABP4, BTBD17, HAGHL, SGSM2, CDK2AP2, CNPY4, DMD, METTL7A, WNK1, PIK3C2A, MTTP, METTL15, CTSA, ARHGEF6, VWA3B, COL11A1, ITGAV, PHYKPL, RNF213, HEG1, GMPR2, NOTCH2, RFX3, DNASE2, RP11-140K17.3, ACP2, ALDH6A1, LRRFIP2, MPP5, TRIL, SNAP23, FAM120A, PRKD1, SALL1, TAF13, ANTXR1, CERS1, TMEM42, NUCB1, UBTD1, RGCC, TMEM189, CERS4, CYFIP1, DENND6B, FBXW9, CABIN1, VEGFB, SDSL, HS2ST1, SHISA4, DNAJC10, REST, CCDC144A, SLC27A5, EEA1, ORMDL2, DLG5, SLC4A4, SC5D, UNC5B, RCAN1, NF1, BHLHE40, LAMA4, FKBP9, LIX1L, DCAKD, GEM, CAMK2D, RHOG, NAT8L, CMTM3, PROS1, LMO2, TANC2, CSRNP1, MAPK4, AGPAT5, VAT1, AGPAT3, SCRN2, SCRIB, ZMAT1, PTGR2, ANKRD9, PAWR, OSBPL1A, COL4A1, LHPP, GSTM4, AGL, C14orf159, PPP1R16A, TMEM131, SNX13, IQGAP1, RB1, DACH1, COL4A5, PDE4B, ATG4C, SLC25A1, ADGRA3, SHROOM3, MMAB, ORAI3, ARHGEF10, TNIP2, SH3PXD2B, PHKG1, RP11-849I19.1, TYRO3, GSTZ1, ALG13, CTDSP1, GNB4, C9orf3, APCDD1, CISD3, APBB2, CASC10, LINC01184, ERF, FBXL7, CAHM, HEY1, KANK1, FAM135A, FRS2, SLC25A23, KAT2B, IMPACT, FZD7, B4GALNT4, SIL1, ARVCF, B3GAT1, TRIM56, RPP25L, C21orf2, PEAK1, GNS, PREX1, KAZN, SPATA6, MAP4K3, PITPNA, HPS1, FASN, MAML2, KIAA1033, TCF7L2, MCCC2, ADCK4, KIAA1958, TMEM150A, SFT2D2, ARHGEF4, BMP2K, PCYT2, CCDC159, ZDHHC24, SNX21, PPP2R5A, ARHGEF40, MFSD1, NXT2, SPARCL1, TIPARP, PTDSS2, KLHDC8B, TEAD1, TMEM170B, ZBTB33, LINC00467, MMS19, BACE1, LRFN4, LSS, SLC11A2, GPC6, PHLPP1, PIPDX, GPC4, RYK, LNPEP, DESI1, NLGN3, and SOAT1.

In some embodiments of the organoids described herein, a radial glia in an organoid cultured for about 6 months or more is characterized as an organoid cell that overexpresses, as compared to the rest of the organoid cells, at least about the first 1, 2, 3, 4, 5, 6, 7, 8, 9, 10, 11, 12, or all 13 of the following genes: ADM, IGFBP2, AK4, IGFBP5, TGIF1, PTPRZ1, PMP2, SFRP1, PRDX4, PGM1, HES1, SERPINE2, and RGS16.

In some embodiments of the organoids described herein, an outer radial glia in an organoid cultured for about 6 months or more is characterized as an organoid cell that overexpresses, as compared to the rest of the organoid cells, at least about the first 5, 10, 20, 30, 40, 50, 75, 100, 125, 150, 175, 200, 250, 300, 350, 400, 450, 500, or all 512 of the following genes: MT3, C8orf4, ATP1A2, CDO1, CA2, TTYH1, APOE, PEA15, LRRC3B, MLC1, REXO2, PTN, PON2, SLC1A3, TRIM9, TNC, BCAN, PTPRZ1, METRN, CST3, CLU, SCRG1, QKI, ITM2C, VIM, HMGN3, GPM6B, TSPAN3, HOPX, MGST3, BAALC, AQP4, B2M, ITM2B, DDAH1, SNX3, INPP1, ADGRV1, ATP1B2, TSC22D4, DOK5, CSPG5, HIGD1A, ID4, HTRA1, BST2, SEPW1, EDNRB, OAT, HSD17B14, ENHO, SDC2, PLPP3, PSAP, CROT, SAT1, GCSH, TFPI, PBXIP1, MGLL, LITAF, NPC2, BDH2, TMEM132B, SPAG16, ZFHX4, PMP22, ADD3, TIMP2, LSAMP, TMEM47, PDGFRB, CHPF, CYSTM1, DNPH1, PAQR8, DDR1, HES5, TP53TG1, ACYP2, HADHB, PLA2G16, IL33, ABHD3, IGFBP7, ANXA6, NDRG4, ANXA5, FZD8, EEPD1, SLC25A18, HLA-A, NFE2L2, LIMCH1, OBSL1, HSPB1, PLEKHB1, LGALS3BP, PRDX6, C1orf122, RHOA, CHCHD10, C1orf6l, LINC00982, CHPT1, IFI27L2, SPATS2L, DTNA, PDLIM3, CD99, HIGD2A, CD58, UQCR11, F3, FAM107A, GPR137B, SARAF, CYBA, LTBP1, BLVRB, PDLIM5, ADGRG1, NOTCH2, LAMP1, GADD45A, SPTSSA, SCRN1, RHOC, LYRM5, SERPINB6, GNG5, OAF, MT-CO1, RAB6B, PAQR6, LAMTOR4, SALL1, C4orf3, NDUFB5, DKK3, GLUD1, TMEM9B, PPT1, POLR2L, QPRT, FAM69C, REEP5, PAM, LHX2, COX6C, DPP7, S1PR1, VAT1L, BMP7, HSD17B12, COMT, CTSL, WASF3, TLE4, PRDM16, LINC00998, FAM198B, CHMP4B, CSTB, PIR, HEBP1, TMEM132A, TIMP1, SFXN5, COL11A1, ELOVL5, TAGLN2, MYO6, AXL, HLA-E, DHRS4L2, CEND1, HLA-B, HRSP12, GLI3, MSRB2, CYR61, FKBP9, APLP2, FAM3C, C1S, GNAS, FGFR3, RAB31, IGFBP4, RP11-263K19.4, SLCO1C1, TIMP3, SNX5, LTBP3, GPX3, FERMT2, MYEOV2, ACADVL, BORCS7, UBL3, MAPKAPK3, PTGFRN, HLA-C, MRC2, CISD1, NEAT1, ACAA1, SLC9A3R1, LRIG1, ANKRD9, CD164, PGM1, SYT11, FOSB, NPAS3, SQSTM1, PLTP, AIG1, SELT, SPATA20, STXBP3, CEBPD, NDUFA13, SLC35F1, NDUFB3, RAMP1, MPP5, PNKD, YAP1, ITGB8, B4GAT1, LAMB2, ARAP2, ZFYVE21, RP3-325F22.5, SPRY2, HDDC2, BCAP29, SDHC, C16orf74, DAG1, LRP4, NDUFB1, FGFR1, LAMP2, TFAP2C, HAGHL, HES4, PCBD1, FAT1, CREM, TMED10, TMEM163, SALL2, LRP10, ATP6V0E1, FOXK1, SEMA5A, CD81, NAA38, HINT2, MYL12A, DAAM2, VAMPS, CRYL1, PDLIM2, OLFM2, ROMO1, GAS2L1, PCGF5, TMED1, KLHDC9, MMP15, TRAPPC6A, TPP1, NME3, GPR37L1, PDHB, SDSL, GSTK1, PPP2CB, FEZ2, GULP1, SEMA6A, KTN1, NAT8L, SFT2D1, C1orf54, LRP1, MMP14, SHROOM3, TM7SF2, RP11-431M7.3, ITPR2, COPRS, CLTC, ST5, ATP1A1, MOXD1, MAPK1, NEK6, CYHR1, LPAR4, SORL1, NRG1, ASAH1, QDPR, C2orf72, P4HTM, CTD-233602.1, CDH4, ZMAT3, ASTN1, GAB1, NT5C, SCARA3, ISCA2, NRBP2, EMID1, LIFR, CNP, EPDR1, TMEM98, TP53I3, TCEAL3, MRPS28, FAM199X, DECR1, RNF213, TMEM59L, GNPTG, GSTO1, TAPBP, THSD1, GEM, CA12, UG0898H09, ITGAV, RIT1, RHPN1, B9D1, CREB5, EFEMP2, ZDHHC2, JAM3, DENND5A, ITGA6, PRCP, PHLPP1, ABCD3, RHPN2, GNG12, GPC6, TSPAN6, CH17-189H20.1, VEGFB, KAZN, PLCD3, METTL7B, MPV17, COL4A5, LPP, MIF4GD, TMEM134, USF2, LIX1L, HEATR5A, PPP2R5A, TRIP6, NQO1, CTD-3252C9.4, CHCHD5, FAM213A, ROM1, SCD, ATP6V1C1, PEX2, TAF13, TMEM179B, DNASE2, GRN, PLCE1, SDC3, MYL5, RARRES3, PRUNE2, TMED5, SPARC, WDR41, NACC2, BICD1, RHOQ, PRKD1, FAM84B, FAM173A, ADAMS, NDP, UBTD1, RENBP, PTPMT1, RFXANK, SGSM2, SSFA2, IMPA1, GRIN2A, ACP2, COA5, TTYH3, RAB9A, REST, S100A16, AHNAK, TMBIM4, PVRL2, MMP24-AS1, CDC42EP1, PDZD11, SOAT1, ADGRB2, MORN2, SLC20A1, CTSD, CTSB, GLIPR2, FADS2, SLC27A1, MAGT1, MOCS2, TMEM205, RP11-410L14.2, C21orf62, CCL2, B3GAT1, PSMB8, ACAT2, AIF1L, ARRDC4, CAST, UROD, DNAJC1, PEPD, PRNP, RP11-140K17.3, CARD19, ACTN1, SCRIB, CAMK2D, HEXB, GLUL, SLC2A8, S100A13, PDXK, IVD, RASSF4, PAWR, PLEC, PLAT, L3HYPDH, SHISA4, PEX10, KRCC1, MSN, ANOS1, TNFRSF1A, NIPSNAP3A, CISD3, SLC16A9, TNFRSF12A, RRAGD, IRS2, COLGALT2, CTSA, WLS, RGS20, SLC27A5, INPPL1, LMO2, SPARCL1, ERF, SLC44A2, NUDT22, SMPD1, NRCAM, RGS3, SWI5, FAM84A, SLC35F5, GLB1, AGTRAP, CFI, RAB29, RGL2, TRIB2, ZDHHC12, HS2ST1, PREX1, ID1, SREBF2, ID3, OSGIN2, SEL1L3, IL6ST, REEP3, CH17-340M24.3, CD44, SIPA1L1, RCAN1, H2AFJ, HABP4, EFHD2, and GLMP.

In some embodiments of the organoids described herein, an outer radial glia/astroglia in an organoid cultured for about 6 months or more is characterized as an organoid cell that overexpresses, as compared to the rest of the organoid cells, at least about the first 5, 10, 20, 30, 40, 50, 75, 100, 125, 150, 175, 200, 250, 300, 350, 400, 450, 500, 550, or all 562 of the following genes: CRYAB, NCAN, BCAN, VIM, HES1, PRDX6, CLU, SAT1, TRIMS, TTYH1, SOX3, AQP4, SOX9, METRN, LIMCH1, HMGN3, TSC22D4, ID4, CDO1, DOK5, RFXANK, ID1, C1orf6l, AIF1L, PEA15, IGFBP5, EZR, LRRC3B, QKI, ZFHX4, ZFP36L1, PTPRZ1, MYO10, GCA, CNN3, NOTCH2, ADGRV1, TIMP2, GLI3, CYBA, GTF2F2, RHOC, PHYHIPL, HSPB1, LIPG, CYR61, CTSH, IFI27L2, DDAH1, GFAP, SLC35F1, ANXA1, SCRN1, PDLIM4, FAM84B, TSPAN3, FSTL1, NPC2, MLC1, HIGD1A, FAM107A, ALDOC, SCRG1, LITAF, SEC11A, PSAT1, GCSH, CSTB, NME4, SOX6, CSPG5, SH3BGRL, CAMTA1, LINC00998, CHCHD5, FEZ2, ATP1B2, TM7SF2, HTRA1, BAALC, COMT, IFITM3, DNPH1, COLGALT2, CREB5, FAM198B, NDRG2, OXA1L, KTN1, SEMA6A, SLC25A5, NINJ1, ANXA6, CYSTM1, HIGD2A, SERF2, TKT, PDLIM2, LHX2, SLC1A3, NKAIN3, S100A16, NDUFA13, SYT11, SESN3, SLC25A18, LRIG1, PSAP, ARHGAP5, HADHB, ITGB8, RPL22L1, CD63, POLR2L, IGFBP4, BMP7, CD151, PON2, NAV1, SDCBP, APC, AKR7A2, SPAG16, TRAPPC6A, FKBP10, URM1, ATP1A2, CNP, ASPH, DAAM2, APRT, TRIP6, TAGLN2, UG0898H09, MMP24-AS1, HINT2, SLC16A9, SEPT9, GRIN2A, OAT, NTSC, GNAI2, PLPP3, C1orf122, GSTK1, OGFRL1, FLNA, PDLIM5, FGFR3, IFI44L, CST3, PAG1, PCBD1, ITM2B, GPR137B, NME3, REEP5, TMEM132B, WDR1, LAMP2, COL11A1, ST5, LSAMP, APOA1BP, CIB1, C8orf4, TP53I3, PMP22, HMGCS1, PDLIM7, GNG12, MSMO1, TMEM47, MSI1, COPRS, UQCR11, DACH1, CAMK2D, TMEM134, TFAP2C, PAQR8, LGALS3BP, BICD1, LINC00982, EEPD1, ALDH3A2, ZFP36, LPAR4, LRRC16A, KCNN3, REXO2, HES5, PRDM16, BLVRB, RP11-126K1.6, IL33, CARD19, EVA1C, UBE2H, SFRP1, TMEM179B, CA2, PALLD, GRN, MTRNR2L1, APOE, LRP10, MTTP, SLC9A3R1, NFE2L2, ENHO, MAPK1, ACAA1, ACAA2, ACOX1, ALDH2, MT-CO2, MINOR, FOXK1, SPRY2, LDB2, TPP1, PBXIP1, TOX, PEX10, LIFR, CYHR1, POLE4, SALL1, CTNND1, SLC25A39, DHRS4L2, MACROD1, PHGDH, RP11-76114.1, MYL5, TMEM131, MSN, PELI2, DNAJC1, NOTCH1, SNX17, BOC, HEY1, CRB2, HEPN1, SNX5, NACC2, MSRB2, KLHL21, FAM69C, PLTP, NDRG4, RAB31, VAMPS, P4HTM, ADD3, MMP15, ACTN1, RAB11B-AS1, ERBB2IP, UBL3, RIT1, ITGA7, REEP3, ARNT2, PDLIM3, VCL, HAGHL, ABAT, ARHGAP12, TAF13, WDR6, FGFR1, TMEM170A, CDC42EP1, STON2, ARRDC4, SFXN5, METTL7A, OSGIN2, CEND1, DKK3, POLR3H, USF2, BST2, GALK1, LTBP1, SLC27A5, IVD, ADAMS, DOCK7, C21orf62, MOXD1, TMEM141, GMPR2, SSFA2, FGFR2, PHLPP1, PLCE1, SOD2, GULP1, PLCD3, GAS2L1, GEM, CTD-233602.1, CBY1, FAM120A, PAM, RAB6B, ROM1, ECI1, LTBP3, TTYH3, NPAS3, PTPN11, WASF3, GLUD1, PLEKHB1, PPT1, OAF, HSD17B14, HRSP12, B9D1, S100A13, MMP14, PDGFD, AXL, CREM, RP11-263K19.4, PAXIP1-AS1, CHPT1, DAG1, ACYP2, MGLL, TP53TG1, RHOQ, FBXO32, RPP25L, HOMER3, FAM134B, GSTZ1, NEK6, DENND5A, NUDT22, MAPK8IP1, HEPACAM, KLHDC8B, SERPINB6, NAT8L, SLCO1C1, RFTN2, FAM84A, IFT57, CD38, GPR37L1, BDH2, S1PR1, HIST2H2BE, ATP2B4, PDCD4, SDSL, PIR, LGALS3, SOX21, C2orf72, CITED1, TCTN1, FGFBP3, GYG1, NRG1, YAP1, FLCN, ALDH6A1, TIMP3, INSIG1, SELENBP1, FZD8, PREX1, AKR1C3, E2F5, SCRN2, TRPS1, SAMD4A, PLA2G16, SLC25A23, PLXNB1, STAT1, CTSA, MGAT4C, NADK2, IQGAP2, TMED1, NMD3, TAPBP, RAI14, CROT, BTBD17, PSMB8, INPP1, HEBP1, DUSP3, RP11-25K19.1, EPHX1, SHISA4, JAM3, OBSL1, TCF7L2, OPHN1, KCNG1, DCAF8, TANC2, KRCC1, SHC1, PPM1K, GNPTG, KCNJ10, BMP2K, KAZN, PAQR6, PTGFRN, FBXO30, FAM199X, ADAM15, ACTR3B, RP11-410L14.2, HSDL2, MIDI, ARAP2, MMAB, ERF, RP11-431M7.3, ZDHHC12, DNASE2, MAPKAPK3, FAT1, PLP1, RNF213, AHNAK, PLEC, PGM1, LRRC1, NR2E1, PAWR, CTD-3252C9.4, MARVELD1, RHPN1, ZDHHC2, RAB9A, B3GALNT2, PIK3C2A, EVI5, HEATR5A, REST, SPATA6, C16orf74, IL6ST, PITPNA, S1PR3, MTSS1L, NAPEPLD, EFHC1, TMEM189, SHROOM3, CLCN7, RP11-140K17.3, LAMB2, MPP5, PVRL2, SLC7A11, PDGFRB, L3HYPDH, MAP4K3, TBC1D1, NEDD9, FKBP9, LSS, CISD3, ITGA6, CD58, SEPN1, HHLA3, SOAT1, SPRED1, MAP3K5, STXBP3, AGTRAP, MRC2, EFEMP2, SLC2A8, ASTN1, RBM38, AP1B1, ANKRD9, ZHX3, WWC1, INPPL1, CXCL12, MAGT1, RP3-325F22.5, MRPS28, ZBTB4, ABHD3, ZMAT1, SIPA1L1, MOB1B, UBTD1, ANOS1, PHF10, CCDC159, PCGF5, PPP2R5A, AEBP1, TFE3, GPC6, SGSM2, CHST3, SNTA1, FAM102A, ABHD17C, RGS3, PHYH, EFHD1, C1orf53, SYNGR1, COL4A5, WLS, SCRIB, AASS, LAMA4, PRKD1, HPS1, C5orf56, ORAI3, TMEM163, ERBB2, MBNL2, AGPAT3, NECAB3, GSTM4, SPATA20, ACP6, NR1D2, KLHDC9, LRRCC1, CTDSP1, PDPN, PHKG1, AAED1, EMID1, LRTOMT, C6orf120, MFSD14B, APBB2, CYBRD1, C1orf194, CNPY4, SNX21, VCAM1, NRBP2, FNDC3B, and TFPI.

In some embodiments corticofugal projection neurons are characterized as cells expressing BCL11B, CRYM, and TLE4 marker genes. In some embodiments, callosal projection neurons are characterized as cells expressing SATB2, INHBA, and FRMD4B marker genes. In some embodiments, interneurons are characterized as cells expressing DLX1, DLX2, and GAD2 marker genes. In some embodiments, outer radial glia are characterized as cells expressing HOPX, TNC and LGALS3 marker genes. In some embodiments, intermediate progenitor cells are characterized as cells expressing EOMES, PPP1R17, and TMEM158 marker genes. In some embodiments, cycling precursors are characterized as cells expressing MKI67, TOP2A, and BIRC5 marker genes.

The organoid may be derived from cells of any suitable species. In some embodiments, the organoid is derived from mammalian cells. In some embodiments, the organoid is derived from human or rodent (e.g., mouse) cells (e.g., human or rodent stem cell or pluripotent cell). In some embodiments, the organoid is derived from human cells. In some embodiments, the organoid is derived from cells (e.g., human cells or rodent cells, human or rodent stem cells or progenitor cells) comprising a mutation associated with a neurological disease or condition.

The neurological disease or condition is not limited. The neurological disease or condition may be any disease affecting synaptic function, neuronal network activity and/or stimulation. As used herein, “neurological disease or condition” refer to neurodegenerative disorders, neuropsychiatric disorders and/or neurodevelopmental disorders. “Neurological disease or condition” also refers to neurological, neuropsychological, neuropsychiatric, neurodegenerative, or neuropsychopharmacological diseases. A neurological disease or condition may be any disease affecting neuronal network connectivity, synaptic function and activity. A neurological disease or condition may be a disease condition involving neural loss mediated or characterized at least partially by at least one of deterioration of neural stem cells and/or progenitor cells. Non-limiting examples of neurodegenerative disorders include polyglutamine expansion disorders (e.g., HD, dentatorubropallidoluysian atrophy, Kennedy's disease (also referred to as spinobulbar muscular atrophy), and spinocerebellar ataxia (e.g., type 1, type 2, type 3 (also referred to as Machado-Joseph disease), type 6, type 7, and type 17), other trinucleotide repeat expansion disorders (e.g., fragile X syndrome, fragile XE mental retardation, Friedreich's ataxia, myotonic dystrophy, spinocerebellar ataxia type 8, and spinocerebellar ataxia type 12), Alexander disease, Alper's disease, Alzheimer disease, amyotrophic lateral sclerosis (ALS), ataxia telangiectasia, Batten disease (also referred to as Spielmeyer-Vogt-Sjogren-Batten disease), Canavan disease, Cockayne syndrome, corticobasal degeneration, Creutzfeldt-Jakob disease, Guillain-Barré syndrome, ischemia stroke, Krabbe disease, kuru, Lewy body dementia, multiple sclerosis, multiple system atrophy, non-Huntingtonian type of Chorea, Parkinson's disease, Pelizaeus-Merzbacher disease, Pick's disease, primary lateral sclerosis, progressive supranuclear palsy, Refsum's disease, Sandhoff disease, Schilder's disease, spinal cord injury, spinal muscular atrophy (SMA), SteeleRichardson-Olszewski disease, and Tabes dorsalis.

In certain contexts, neurodegenerative disorders encompass neurological injuries or damages to the CNS or the PNS associated with physical injury (e.g., head trauma, mild to severe traumatic brain injury (TBI), spinal cord injury, diffuse axonal injury, craniocerebral trauma, cranial nerve injuries, cerebral contusion, intracerebral haemorrhage and acute brain swelling), ischemia (e.g., resulting from spinal cord infarction or ischemia, ischemic infarction, stroke, cardiac insufficiency or arrest, atherosclerotic thrombosis, ruptured aneurysm, embolism or hemorrhage), certain medical procedures or exposure to biological or chemic toxins or poisons (e.g., surgery, coronary artery bypass graft (CABG), electroconvulsive therapy, radiation therapy, chemotherapy, anti-neoplastic drugs, immunosuppressive agents, psychoactive, sedative or hypnotic drugs, alcohol, bacterial or industrial toxins, plant poisons, and venomous bites and stings), tumors (e.g., CNS metastasis, intraaxial tumors, primary CNS lymphomas, germ cell tumors, infiltrating and localized gliomas, fibrillary astrocytomas, oligodendrogliomas, ependymomas, pleomorphic xanthoastrocytomas, pilocytic astrocytomas, extraaxial brain tumors, meningiomas, schwannomas, neurofibromas, pituitary tumors, and mesenchymal tumors of the skull, spine and dura matter), infections (e.g., bacterial, viral, fungal, parasitic or other origin is selected from the group consisting of pyrogenic infections, meningitis, tuberculosis, syphilis, encephalomyelitis and leptomeningitis), metabolic or nutritional disorders (e.g., glycogen storage diseases, acid lipase diseases, Wemicke's or Marchiafava-Bignami's disease, Lesch-Nyhan syndrome, Farber's disease, gangliosidoses, vitamin B12 and folic acid deficiency), cognition or mood disorders (e.g., learning or memory disorder, bipolar disorders and depression), and various medical conditions associated with neural damage or destruction (e.g., asphyxia, prematurity in infants, perinatal distress, gaseous intoxication for instance from carbon monoxide or ammonia, coma, hypoglycaemia, dementia, epilepsy and hypertensive crises).

“Neuropsychiatric disorder” encompasses mental disorders attributable to diseases of the nervous system. Non-limiting examples of neuropsychiatric disorders include addictions, childhood developmental disorders, eating disorders, degenerative diseases, mood disorders, neurotic disorders, psychosis, sleep disorders, depression, obsessive-compulsive disorder, schizophrenia, visual hallucination, auditory hallucination, eating disorder, bipolar disorder, epilepsy, autism spectrum disorder (ASD), and amyotrophic lateral sclerosis (ALS).

Stem cells and progenitor cells used to derive the organoids described herein can be any cells derived from any kind of tissue (for example embryonic tissue such as fetal or pre-fetal tissue, or adult tissue), which stem cells have the characteristic of being capable under appropriate conditions of producing progeny of different cell types, e.g. derivatives of all of at least one of the 3 germinal layers (endoderm, mesoderm, and ectoderm). These cell types may be provided in the form of an established cell line, or they may be obtained directly from primary embryonic tissue and used immediately for differentiation. Included are cells listed in the NIH Human Embryonic Stem Cell Registry, e.g. hESBGN-01, hESBGN-02, hESBGN-03, hESBGN-04 (BresaGen, Inc.); HES-1, HES-2, HES-3, HES-4, HES-5, HES-6 (ES Cell International); Miz-hES1 (MizMedi Hospital-Seoul National University); HSF-1, HSF-6 (University of California at San Francisco); and H1, H7, H9, H13, H14 (Wisconsin Alumni Research Foundation (WiCell Research Institute)).

In some embodiments, the stem cells can be isolated from tissue including solid tissue. In some embodiments, the tissue is skin, fat tissue (e.g. adipose tissue), muscle tissue, heart or cardiac tissue. In other embodiments, the tissue is for example but not limited to, umbilical cord blood, placenta, bone marrow, or chondral.

Stem cells of interest also include embryonic cells of various types, exemplified by human embryonic stem (hES) cells, described by Thomson et al. (1998) Science 282:1145; embryonic stem cells from other primates, such as Rhesus stem cells (Thomson et al. (1995) Proc. Natl. Acad. Sci. USA 92:7844); marmoset stem cells (Thomson et al. (1996) Biol. Reprod. 55:254); and human embryonic germ (hEG) cells (Shambloft et al., Proc. Natl. Acad. Sci. USA 95:13726, 1998). Also of interest are lineage committed stem cells, such as mesodermal stem cells and other early cardiogenic cells (see Reyes et al. (2001) Blood 98:2615-2625; Eisenberg & Bader (1996) Circ Res. 78(2):205-16; etc.) The stem cells may be obtained from any mammalian species, e.g. human, equine, bovine, porcine, canine, feline, rodent, e.g. mice, rats, hamster, primate, etc.

Embryonic stem (ES) cells are considered to be undifferentiated when they have not committed to a specific differentiation lineage. Such cells display morphological characteristics that distinguish them from differentiated cells of embryo or adult origin. Undifferentiated ES cells are easily recognized by those skilled in the art, and typically appear in the two dimensions of a microscopic view in colonies of cells with high nuclear/cytoplasmic ratios and prominent nucleoli. Undifferentiated ES cells express genes that may be used as markers to detect the presence of undifferentiated cells, and whose polypeptide products may be used as markers for negative selection. For example, see U.S. application Ser. No. 2003/0224411 A1; Bhattacharya (2004) Blood 103(8):2956-64; and Thomson (1998), supra., each herein incorporated by reference. Human ES cell lines express cell surface markers that characterize undifferentiated nonhuman primate ES and human EC cells, including stage-specific embryonic antigen (SSEA)-3, SSEA-4, TRA-1-60, TRA-1-81, and alkaline phosphatase. The globo-series glycolipid GL7, which carries the SSEA-4 epitope, is formed by the addition of sialic acid to the globo-series glycolipid GbS, which carries the SSEA-3 epitope. Thus, GL7 reacts with antibodies to both SSEA-3 and SSEA-4. The undifferentiated human ES cell lines did not stain for SSEA-1, but differentiated cells stained strongly for SSEA-I. Methods for proliferating hES cells in the undifferentiated form are described in WO 99/20741, WO 01/51616, and WO 03/020920.

A mixture of cells from a suitable source of endothelial, muscle, and/or neural stem cells can be harvested from a mammalian donor by methods known in the art. A suitable source is the hematopoietic microenvironment. For example, circulating peripheral blood, preferably mobilized (i.e., recruited), may be removed from a subject. Alternatively, bone marrow may be obtained from a mammal, such as a human patient, undergoing an autologous transplant. In some embodiments, stem cells can be obtained from the subject's adipose tissue, for example using the CELUTION™ SYSTEM from Cytori, as disclosed in U.S. Pat. Nos. 7,390,484 and 7,429,488 which are incorporated herein in their entirety by reference.

In some embodiments, the stem cells can be reprogrammed stem cells, such as stem cells derived from somatic or differentiated cells. In such an embodiment, the de-differentiated stem cells can be for example, but not limited to, neoplastic cells, tumor cells and cancer cells or alternatively induced reprogrammed cells such as induced pluripotent stem cells or iPS cells.

In some embodiments, the organoid is derived from PGP1 (Personal Genome Project 1) hiPSC (human induced pluripotent stem cells); HUES66 hESC (human embryonic stem cells); 11a hiPSC; GM08330 hiPSC (also known as GM8330-8); or Mito 210 hiPSC.

### Methods of Manufacturing Dorsal Forebrain Organoids

As discussed in detail in the examples section, the methods of the invention for producing dorsal forebrain organoids surprisingly produced organoids from different HuESCs and iPSCs each having consistent cell types and cell proportions. The ability to produce organoids with highly similar make-up is of great value for study of brain development and for screening for neurologically active agents.

Illustrative methods for molecular genetics and genetic engineering that may be used in the technology described herein may be found, for example, in current editions of Molecular Cloning: A Laboratory Manual, (Sambrook et al., Cold Spring Harbor); Gene Transfer Vectors for Mammalian Cells (Miller & Calos eds.); and Current Protocols in Molecular Biology (F. M. Ausubel et al. eds., Wiley & Sons). Cell biology, protein chemistry, and antibody techniques can be found, for example, in Current Protocols in Protein Science (J. E. Colligan et al. eds., Wiley & Sons); Current Protocols in Cell Biology (J. S. Bonifacino et al., Wiley & Sons) and Current protocols in Immunology (J. E. Colligan et al. eds., Wiley & Sons.). Illustrative reagents, cloning vectors, and kits for genetic manipulation may be commercially obtained, for example, from BioRad, Stratagene, Invitrogen, ClonTech, and Sigma-Aldrich Co.

Suitable cell culture methods may be found, or described generally, in the current edition of Culture of Animal Cells: A Manual of Basic Technique (R. I. Freshney ed., Wiley & Sons); General Techniques of Cell Culture (M. A. Harrison & I. F. Rae, Cambridge Univ. Press), and Embryonic Stem Cells: Methods and Protocols (K. Turksen ed., Humana Press). Suitable tissue culture supplies and reagents are commercially available, for example, from Gibco/BRL, Nalgene-Nunc International, Sigma Chemical Co., and ICN Biomedicals.

Pluripotent stem cells can be propagated by one of ordinary skill in the art and continuously in culture, using culture conditions that promote proliferation without promoting differentiation. Exemplary serum-containing ES medium is made with 80% DMEM (such as Knock-Out DMEM, Gibco), 20% of either defined fetal bovine serum (FBS, Hyclone) or serum replacement (WO 98/30679), 1% non-essential amino acids, 1 mM L-glutamine, and 0.1 mM β-mercaptoethanol. Just before use, human bFGF is added to 4 ng/mL (WO 99/20741, Geron Corp.). Traditionally, ES cells are cultured on a layer of feeder cells, typically fibroblasts derived from embryonic or fetal tissue.

Puripotent SCs can be maintained in an undifferentiated state even without feeder cells. The environment for feeder-free cultures includes a suitable culture substrate, particularly an extracellular matrix such as MATRIGEL® or laminin. Typically, enzymatic digestion is halted before cells become completely dispersed (about 5 min with collagenase IV). Clumps of about 10 to 2,000 cells are then plated directly onto the substrate without further dispersal.

Feeder-free cultures are supported by a nutrient medium containing factors that support proliferation of the cells without differentiation. Such factors may be introduced into the medium by culturing the medium with cells secreting such factors, such as irradiated (about 4,000 rad) primary mouse embryonic fibroblasts, telomerized mouse fibroblasts, or fibroblast-like cells derived from pPS cells. Medium can be conditioned by plating the feeders at a density of about 5-6×10⁴ cm⁻² in a serum free medium such as KO DMEM supplemented with 20% serum replacement and 4 ng/mL bFGF. Medium that has been conditioned for 1-2 days is supplemented with further bFGF, and used to support pluripotent SC culture for 1-2 days. Features of the feeder-free culture method are further discussed in International Patent Publication WO 01/51616; and Xu et al., Nat. Biotechnol. 19:971, 2001.

Under the microscope, ES cells appear with high nuclear/cytoplasmic ratios, prominent nucleoli, and compact colony formation with poorly discernable cell junctions. Primate ES cells express stage-specific embryonic antigens (SSEA) 3 and 4, and markers detectable using antibodies designated Tra-1-60 and Tra-1-81 (Thomson et al., Science 282:1145, 1998). Mouse ES cells can be used as a positive control for SSEA-1, and as a negative control for SSEA-4, Tra-1-60, and Tra-1-81. SSEA-4 is consistently present in human embryonal carcinoma (hEC) cells. Differentiation of pluripotent SCs in vitro results in the loss of SSEA-4, Tra-1-60, and Tra-1-81 expression, and increased expression of SSEA-1, which is also found on undifferentiated hEG cells.

In some embodiments, thawing, maintenance, and passaging of human pluripotent stem cells are performed by the methods described in Arlotta, P. et al. Long-term culture and electrophysiological characterization of human brain organoids, Protocol Exchange https://dx.doi.org/10.1038/protex.2017.049 (2017), incorporated herein by reference.

Some aspects of the present disclosure are related to a method of producing a dorsal forebrain organoid, comprising obtaining a dorsal forebrain marker-positive organoid by a first step comprising culturing an aggregate of pluripotent stem cells in suspension in the presence of a Wnt signal inhibitor and a TGFβ signal inhibitor, and a second step comprising culturing the dorsal forebrain progenitor marker-positive aggregate in a spinner flask at about 20% oxygen (e.g., atmospheric oxygen levels) and 5% CO₂. In some embodiments, the method of producing a dorsal forebrain organoid is the method shown in the “detailed protocol” section shown below.

The pluripotent stem cells are not limited and may be any stem cell or pluripotent cell described herein. In some embodiments, the cells are from cell line PGP1 hiPSC; HUES66 hESC; 11a hiPSC; GM08330 hiPSC (also known as GM8330-8); or Mito 210 hiPSC. In some embodiments, the cells are induced pluripotent stem cells from a subject having a neurological disease or condition as described herein.

The dorsal forebrain markers are not limited and may be any markers described herein. In some embodiments, the markers may be markers described for DFOs cultured for 1 month, 3 months, 6 months, or longer as described herein. In some embodiments, the markers comprise PAX6 and MAP2. In some embodiments, the markers comprise PAX6, MAP2, and EMX1.

Any suitable method may be used to culture an aggregate of pluripotent stem cells in suspension. In some embodiments, stem cells are dissociated into single cells and then cultured in low attachment tissue culture plates, spinner flasks, or aggrewell plates. In some embodiments, the cells are disassociated in the presence of a ROCK inhibitor (e.g., Y-27632). In some embodiments, the dissociated cells are cultured in cortical differentiation medium (e.g., CDM mediums I-II as described in the “detailed protocol” below). In some embodiments, the cortical differentiation medium (CDM) is serum free. In some embodiments, the cortical differentiation medium is further supplemented with a ROCK inhibitor (e.g., Y-27632). In some embodiments, the CDM is supplemented with a ROCK inhibitor for about the first 6 days of culture.

The Wnt signal inhibitor and the TGFβ signal inhibitor are not limited and may be any suitable inhibitors known in the art. In some embodiments, the TGFβ signal inhibitor is SB431542 (e.g., SB431542 to a final concentration of about 5 μM). In some embodiments, the Wnt signal inhibitor IWR1 (e.g., IWR1 to a final concentration of 3 μM).

In some embodiments, the cells are cultured for about 16-20 day (e.g., 18 days) in 96 v-well low attachment plates (e.g., prime surface 96V plates), thereby forming aggregates. In some embodiments, the cells are cultured at a concentration of about 8000-10,000 (e.g., 9000) cells per well in a volume of about 100 μl. In some embodiments, the cells are cultured at 37° C. and 5% CO₂. In some embodiments, the cells are cultured without shaking. During culturing, the CDM media should be changed/replenished as needed (see, e.g., “detailed protocol”). In some embodiments, the CDM media is changed about every three days.

In some embodiments, after culturing for about 16-20 day (e.g., 18 days), the cell aggregates are transferred to 100 mm ultra-low attachment tissue culture plates and further cultured with CDM media (e.g., CDM II media as described herein in the “detailed protocol”). In some embodiments, the CDM media comprises N-2 supplement. During culturing, the CDM media should be changed/replenished as needed (see, e.g., “detailed protocol”). In some embodiments, the CDM media is changed about every three days. In some embodiments, the CDM media does not comprise a Wnt signal inhibitor or a TGFβ signal inhibitor. In some embodiments, about 40-60 (e.g., about 48) aggregates are transferred into a 100 mm ultra-low attachment tissue culture plate with about 15 ml of media. In some embodiments, the aggregates are cultured in the tissue culture plates at 37° C. and 5% CO₂ for about 15-20 days (e.g., 17 days). In some embodiments, the aggregates are cultured with shaking (e.g., on an orbital shaker). In some aspects, the rotation rate of the orbital shaker is about 5 RPM, 10 RPM, 15 RPM, 20 RPM, 25 RPM, 30 RPM, 35 RPM, 40 RPM, 45 RPM, 50 RPM, 55 RPM, 60 RPM, 65 RPM, 70 RPM, 75 RPM, 80 RPM, 85 RPM, 90 RPM, 95 RPM, 100 RPM, 105 RPM, 110 RPM, 115 RPM, 120 RPM, 125 RPM, 130 RPM, 135 RPM, 140 RPM, 145 RPM, or 150 RPM. In some aspects, the rotation rate of the orbital shaker is a rate that allows sufficient oxygen diffusion in the medium and at the same time preserves the integrity of the aggregates. In some aspects, the rotation rate of the orbital shaker that allows enough oxygen diffusion in the medium and at the same time preserves the integrity of the aggregates is about 60-80 rpm, preferably about 70 rpm.

In some embodiments, after culturing for about 30-40 days (e.g., 35 days), the cell aggregates may be transferred to a spinner flask. In some embodiments, culturing cell aggregates for about 30-40 days as detailed herein produces DFOs as described herein (i.e., DFOs cultured for about a month). In some embodiments, about 90-100 cell aggregates (now organoids) are added to a 125 ml spinner flask containing about 100 ml of CDM media. In some embodiments, the CDM media comprises serum (e.g., fetal bovine serum). In some embodiments, the CDM media comprises heparin. In some embodiments, the CDM media comprises N-2 supplement. In some embodiments, the CDM media comprises heparin. In some embodiments, the CDM media is CDM III media as described in the “detailed protocol” below.

In some embodiments, the organoids are cultured in a spinner flask at 37° C. and 5% CO₂ with stirring. In some embodiments, the stirring speed is about 30 RPM, 35 RPM, 40 RPM, 45 RPM, 50 RPM, 51 RPM, 52 RPM, 53 RPM, 54 RPM, 55 RPM, 56 RPM, 57 RPM, 58 RPM, 59 RPM, 60 RPM, 65 RPM, 70 RPM, 75 RPM, or 80 RPM. In some aspects, the stirring is at a speed that allows sufficient oxygen diffusion in the medium and at the same time preserves the integrity of the organoids. In some aspects, the stirring speed that allows enough oxygen diffusion in the medium and at the same time preserves the integrity of the organoids is about 50-60 rpm, preferably about 56 rpm. In some embodiments, the organoids are cultured for about 30-40 days (e.g., 35 days) with media change/replenishment as needed (see, e.g., “detailed protocol”). In some embodiments, the CDM media is changed about every 7 days.

In some embodiments, after about 30-40 days (e.g., 35 days) of culturing in spinner flasks, the formulation of the CDM media is changed (e.g., to CDM IV media as described in “detailed protocol”). In some embodiments, the new CDM media comprises serum (e.g., fetal bovine serum). In some embodiments, the new CDM media comprises heparin. In some embodiments, the new CDM media comprises N-2 supplement. In some embodiments, the new CDM media comprises heparin. In some embodiments, the new CDM media comprises B-27 supplement. In some embodiments, the organoids are cultured in the spinner flask at 37° C. and 5% CO₂ with stirring. The stirring speed is not limited and may be any suitable stirring speed described herein. In some embodiments, the stirring speed is about 56 RPM.

In some embodiments, the organoids may be cultured in a spinner flask for at least about 1, 2, 3, 4, 5, 6, 7, 8, 9, 10, 11, 12, 13, 14, 15, 16, 17, 18, 19, 20, 21, 22, 23, 24 months or more.

In some embodiments, the methods described herein produce the organoids as described throughout. In some embodiments, an organoid produced by culturing via the methods described herein for about between 1 month (e.g., 35 days) and 3 months (e.g., 70 days) comprises one or more of corticofugal projection neurons, callosal projection neurons, cycling progenitors, immature corticofugal projection neurons, immature callosal projection neurons, immature projection neurons, immature interneurons (e.g., immature inhibitory interneurons), intermediate progenitor cells, outer radial glia, Cajal-Retzius neurons, and radial glia. In some embodiments, an organoid produced by culturing via the methods described herein for about between 1 month (e.g., 35 days) and 3 months (e.g., 70 days) comprises immature projection neurons, callosal projection neurons, intermediate progenitor cells, radial glia, and cycling progenitors. In some embodiments, an organoid produced by culturing via the methods described herein for about between 1 month (e.g., 35 days) and 3 months (e.g., 70 days) comprises immature projection neurons, callosal projection neurons, intermediate progenitor cells, radial glia, cycling progenitors, and immature interneurons. The cells comprising the organoid may be characterized by any expression pattern or marker described herein.

In some embodiments, an organoid produced by culturing via the methods described herein for about between 1 month (e.g., 35 days) and 3 months (e.g., 70 days) comprises In some embodiments, an organoid produced by culturing via the methods described herein for about between 1 month (e.g., 35 days) and 3 months (e.g., 70 days) comprises about 17%-28% corticofugal projection neurons, about 40%-50% callosal projection neurons, about 4%-7% cycling progenitors, about 2% or less (including 0%) immature interneurons, about 3%-15% immature projection neurons, about 3%-6% intermediate progenitor cells, about 9%-14% radial glia, and about 0.5% or less (including 0%) of Cajal-Retzius neurons.

In some embodiments, an organoid produced by culturing via the methods described herein for about 6 month or longer comprises one or more of astroglia, callosal projection neurons, cycling progenitors, immature callosal projection neurons, immature interneurons (e.g., immature inhibitory neurons), immature projection neurons, intermediate progenitor cells, outer radial glia, radial glia, outer radial glia/astroglia, ventral precursors, and cycling interneuron precursors (e.g., cycling inhibitory interneuron precursors).

In some embodiments, an organoid produced by culturing via the methods described herein for about 6 months or longer comprises about 6%-16% astroglia. In some embodiments, an organoid produced by culturing via the methods described herein for about 6 months or longer comprises about 7%-22% callosal projection neurons. In some embodiments, an organoid produced by culturing via the methods described herein for about 6 months or longer comprises about 5%-8% cycling progenitors. In some embodiments, an organoid produced by culturing via the methods described herein for about 6 months or longer comprises about 10%-31% immature interneurons. In some embodiments, an organoid produced by culturing via the methods described herein for about 6 months or longer comprises about 2%-10% immature projection neurons. In some embodiments, an organoid produced by culturing via the methods described herein for about 6 months or longer comprises about 1%-7% intermediate progenitor cells. In some embodiments, an organoid produced by culturing via the methods described herein for about 6 months or longer comprises about 22%-39% radial glia. In some embodiments, an organoid produced by culturing via the methods described herein for about 6 months or longer comprises about 4%-8% ventral precursors. In some embodiments, an organoid produced by culturing via the methods described herein for about 6 months or longer comprises substantially no corticofugal projection neurons or immature corticofugal projection neurons.

In some embodiments, an organoid produced by culturing via the methods described herein for about 6 months or longer comprises about 6%-16% astroglia, about 7%-22% callosal projection neurons, about 5%-8% cycling progenitors, about 10%-31% immature interneurons, about 2%-10% immature projection neurons, about 1%-7% intermediate progenitor cells, about 22%-39% radial glia, and about 4%-8% ventral precursors. In some embodiments, an organoid produced by culturing via the methods described herein for about 7, 8, 9, 10, 11, 12, 13, 14, 15, 16, 17, 18, 19, 20, 21, 22, 23, 24 months or longer comprises about 6%-16% astroglia, about 7%-22% callosal projection neurons, about 5%-8% cycling progenitors, about 10%-31% immature interneurons, about 2%-10% immature projection neurons, about 1%-7% intermediate progenitor cells, about 22%-39% radial glia, and about 4%-8% ventral precursors. The cells comprising the organoid may be characterized by any expression pattern or marker described herein.

In some embodiments, the methods described herein produce multiple organoids having highly similar cell types and cell proportions. In some embodiments, the methods described herein produce a plurality of organoids having a mutual information (MI) score of less than about 0.1, less than about 0.09, less than about 0.08, less than about 0.07, less than about 0.06, less than about 0.05, less than about 0.049, less than about 0.045, less than about 0.042, or less than about 0.03. In some embodiments, the MI score for organoids produced after culture for about 3 months is less than about 0.06, 0.05, or 0.049. In some embodiments, the MI score for organoids produced after culture for about 6 months is less than about 0.1, 0.09, or 0.089. In some embodiments, the MI scores have Z-scores (divergence of the MI score for individual organoids from the mean MI score expected at random) of less than 80, less than 70, less than 60, less than 50, less than 50, less than 40, or less than 30. In some embodiments, the z-score for organoids produced after culture for about 3 months is less than about 45.0, 40.0, or 38.0. In some embodiments, the z-score for organoids produced after culture for about 6 months is less than about 85.0, 80.0, or 75.7. In some embodiments, the organoids produced by the methods disclosed herein have an intraclass correlation (ICC) of more than 0.65, more than 0.68, more than 0.70, more than 0.75, more than 0.80, more than 0.85, or more than 0.90. In some embodiments, the ICC for organoids cultured for 3 months by the methods described herein are 0.80 or more (e.g., 0.85 or more). In some embodiments, the ICC for organoids cultured for 6 months or more by the methods described herein are 0.60 or more (e.g., 0.68 or more). Methods of calculating MI, z-score, and ICC are known in the art and are not limited. In some embodiments, the methods of calculating MI, z-score, and ICC are ones described in the examples section.

### Methods of Screening

In some embodiments, the invention provides a method of screening test agents to identify candidate neurologically active agents. In some aspects, dorsal forebrain organoids described herein or generated as described by the methods of the invention are used. In some embodiments, DFOs are generated from cells derived from a subject having a neurological disease or disorder (e.g., a neurodegenerative disorder, such as epilepsy, autism spectrum disorder (ASD), bipolar disorder, schizophrenia or a neurological, neuropsychological, neuropsychiatric, neurodegenerative, or neuropsychopharmacological disease).

Some embodiments are directed to a method of screening for a candidate neurologically active agent, comprising contacting a dorsal forebrain organoid as described herein with a test agent, and assessing changes to the organoid, wherein the test agent is identified as a candidate neurologically active agent when contact with the test agent causes a change to the organoid as compared to a control organoid.

The term “agent” as used herein means any compound or substance such as, but not limited to, a small molecule, nucleic acid, polypeptide, peptide, drug, ion, etc. An “agent” can be any chemical, entity or moiety, including without limitation synthetic and naturally-occurring proteinaceous and non-proteinaceous entities. In some embodiments, an agent is nucleic acid, nucleic acid analogues, proteins, antibodies, peptides, aptamers, oligomer of nucleic acids, amino acids, or carbohydrates including without limitation proteins, oligonucleotides, ribozymes, DNAzymes, glycoproteins, siRNAs, lipoproteins, aptamers, and modifications and combinations thereof etc. In certain embodiments, agents are small molecules having a chemical moiety. For example, chemical moieties include unsubstituted or substituted alkyl, aromatic, or heterocyclyl moieties including macrolides, leptomycins and related natural products or analogues thereof. Compounds can be known to have a desired activity and/or property, or can be selected from a library of diverse compounds.

As used herein, the term “contacting” (i.e., contacting the organoid with an agent) is intended to include incubating the agent and organoid together in vitro. It is understood that the organoid contacted with an agent can also be simultaneously or subsequently contacted with another agent. In some embodiments, the organoid is contacted with at least two, at least three, at least four, at least five, at least six, at least seven, at least eight, at least nine, or at least ten agents.

In some embodiments, one or more parameters of the organoid are measured in response to contact with the test agent. In certain aspects, the one or more parameters include: a) neural (monosynaptic) and/or network (polysnaptic) evoked response; b) individual spike trains (e.g., spike delays, peri-stimulus event histograms, inter spike interval histograms); c) correlations across spike trains and network synchronization; d) network topology (e.g., vertices, edges, path length, clustering, small-worldliness); and/or e) selective pharmacology to study basic physiology (e.g., dissect neural networks) and disease models. For example, spikes (e.g., action potentials) may be isolated in organoids. In some aspects, the presence of distinct firing patterns is identified. In some embodiments, the change is a modulation of stimulus (e.g., contact with the agent) induced activity or spontaneous activity of the organoid. In some embodiments, growth, viability, cellular composition, developmental trajectory, or morphology of the contacted organoid is measured and compared to a control after contact with the test agent.

In some embodiments, the control organoid is an organoid not contacted with the test agent. In some embodiments, the test organoid is derived from a subject having a neurological disease or condition and the control organoid is from a subject without the neurological disease or condition. Thus, in certain embodiments, if contact of the test organoid with the test agent results in the test organoid exhibiting a property more similar to the control organoid, then the agent can be identified as a beneficial agent for the neurological disease or condition.

In some embodiments, a high throughput screen (HTS) is performed. A high throughput screen can utilize the highly reproducible organoids described herein. High throughput screens often involve testing large numbers of compounds with high efficiency, e.g., in parallel. Often such screening is performed in multiwell plates other vessels in which multiple physically separated cavities or depressions are present in a substrate. High throughput screens often involve use of automation, e.g., for liquid handling, imaging, data acquisition and processing, etc. Certain general principles and techniques that may be applied in embodiments of a HTS of the present invention are described in Macarrón R & Hertzberg RP. Design and implementation of high-throughput screening assays. Methods Mol Biol., 565:1-32, 2009 and/or An W F & Tolliday N J., Introduction: cell-based assays for high-throughput screening. Methods Mol Biol. 486:1-12, 2009, and/or references in either of these. Useful methods are also disclosed in High Throughput Screening: Methods and Protocols (Methods in Molecular Biology) by William P. Janzen (2002) and High-Throughput Screening in Drug Discovery (Methods and Principles in Medicinal Chemistry) (2006) by Jorg W\ddot{ν}ser.

The articles “a”, “an” and “the” as used herein, unless clearly indicated to the contrary, should be understood to include the plural referents. Claims or descriptions that include “or” between one or more members of a group are considered satisfied if one, more than one, or all of the group members are present in, employed in, or otherwise relevant to a given product or process unless indicated to the contrary or otherwise evident from the context. It should it be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not in every case been specifically set forth in haec verba herein. It should also be understood that any embodiment of the invention, e.g., any embodiment found within the prior art, can be explicitly excluded from the claims, regardless of whether the specific exclusion is recited in the specification.

As used herein the term “comprising” or “comprises” is used in reference to compositions, methods, and respective component(s) thereof, that are essential to the invention, yet open to the inclusion of unspecified elements, whether essential or not. The term “consisting essentially of” refers to those elements required for a given embodiment. The term permits the presence of additional elements that do not materially affect the basic and novel or functional characteristic(s) of that embodiment of the invention. The term “consisting of” refers to compositions, methods, and respective components thereof as described herein, which are exclusive of any element not recited in that description of the embodiment.

Where ranges are given herein, the invention includes embodiments in which the endpoints are included, embodiments in which both endpoints are excluded, and embodiments in which one endpoint is included and the other is excluded. It should be assumed that both endpoints are included unless indicated otherwise. Furthermore, it is to be understood that unless otherwise indicated or otherwise evident from the context and understanding of one of skill in the art, values that are expressed as ranges can assume any specific value or subrange within the stated ranges in different embodiments of the invention, to the tenth of the unit of the lower limit of the range, unless the context clearly dictates otherwise. It is also understood that where a series of numerical values is stated herein, the invention includes embodiments that relate analogously to any intervening value or range defined by any two values in the series, and that the lowest value may be taken as a minimum and the greatest value may be taken as a maximum. Numerical values, as used herein, include values expressed as percentages. For any embodiment of the invention in which a numerical value is prefaced by “about” or “approximately”, the invention includes an embodiment in which the exact value is recited. For any embodiment of the invention in which a numerical value is not prefaced by “about” or “approximately”, the invention includes an embodiment in which the value is prefaced by “about” or “approximately”. “Approximately” or “about” generally includes numbers that fall within a range of 1% or in some embodiments 5% of a number in either direction (greater than or less than the number) unless otherwise stated or otherwise evident from the context (except where such number would impermissibly exceed 100% of a possible value).

Furthermore, it is to be understood that the invention encompasses all variations, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. Where elements are presented as lists, e.g., in Markush group or similar format, it is to be understood that each subgroup of the elements is also disclosed, and any element(s) can be removed from the group.

Certain claims are presented in dependent form for the sake of convenience, but any dependent claim may be rewritten in independent format to include the limitations of the independent claim and any other claim(s) on which such claim depends, and such rewritten claim is to be considered equivalent in all respects to the dependent claim (either amended or unamended) prior to being rewritten in independent format. It should also be understood that, unless clearly indicated to the contrary, in any methods claimed herein that include more than one act, the order of the acts of the method is not necessarily limited to the order in which the acts of the method are recited, but the invention includes embodiments in which the order is so limited. It is contemplated that all aspects described above are applicable to all different embodiments of the invention. It is also contemplated that any of the above embodiments can be freely combined with one or more other such embodiments whenever appropriate.

All patents, patent applications, and other publications (e.g., scientific articles, books, websites, and databases) mentioned herein are incorporated by reference in their entirety. In case of a conflict between the specification and any of the incorporated references, the specification (including any amendments thereof, which may be based on an incorporated reference), shall control. Standard art-accepted meanings of terms are used herein unless indicated otherwise. Standard abbreviations for various terms are used herein.

The above discussed, and many other features and attendant advantages of the present inventions will become better understood by reference to the following detailed description of the invention.

### EXAMPLES

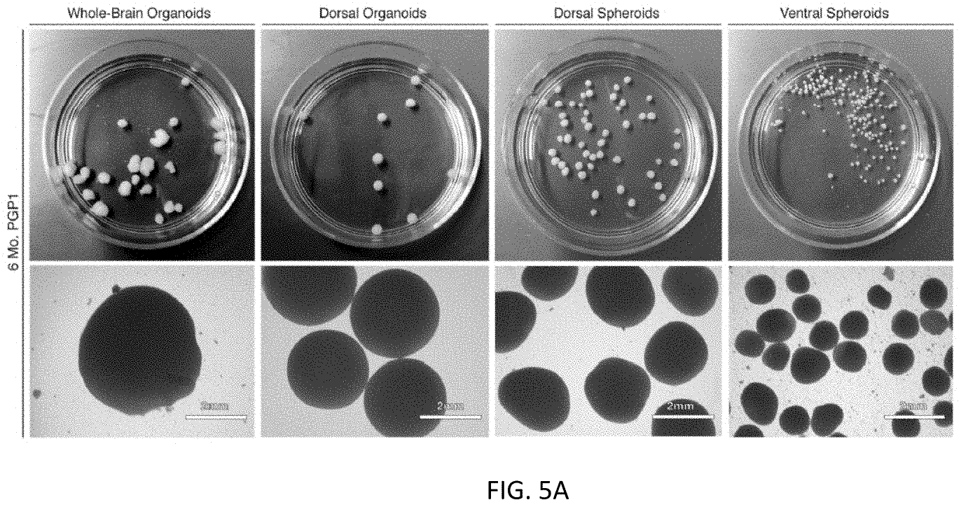

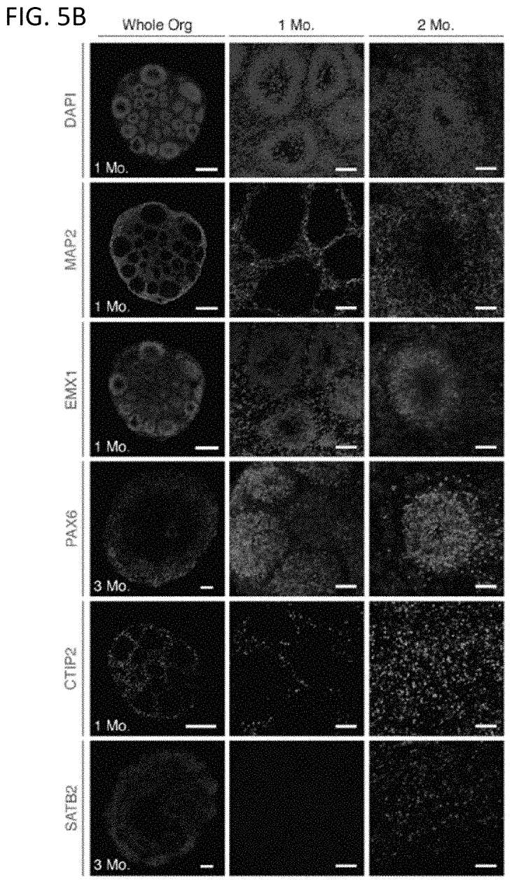

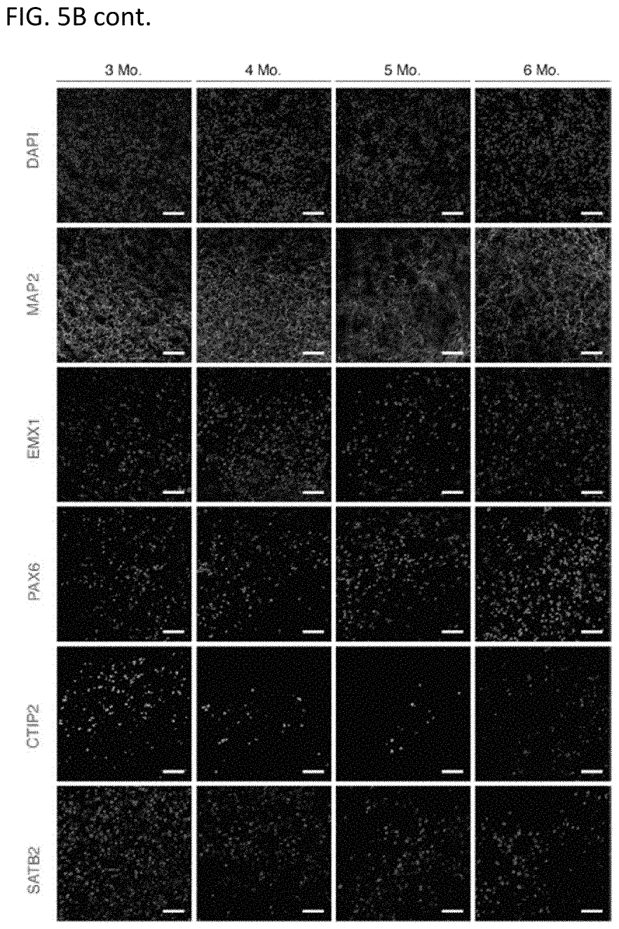

Four distinct protocols for producing 3D brain organoids and spheroids: self-patterned whole-brain organoids³; patterned dorsal forebrain organoids (modified from⁵); and patterned dorsal and ventral spheroids (modified from¹⁵) were established. Each protocol was adapted to growth in spinner-flask bioreactors over durations of at least 6 months, to achieve advanced maturation (FIG. 5A; see Methods for details on protocol modifications).

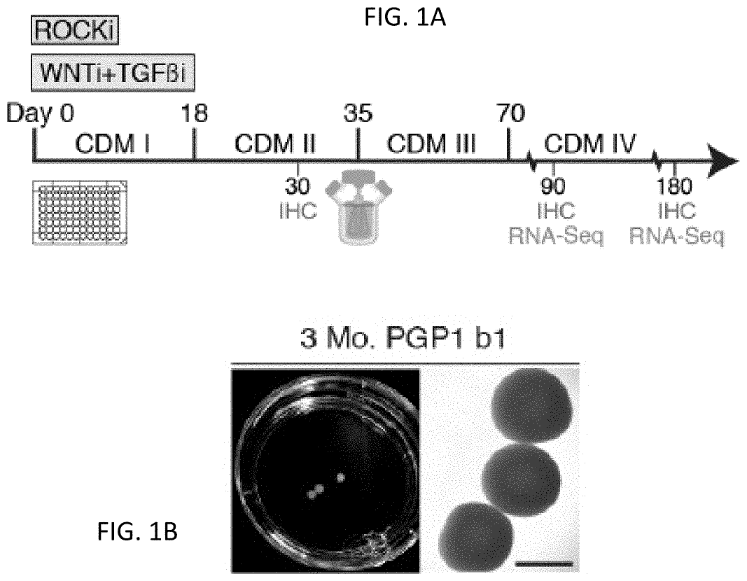

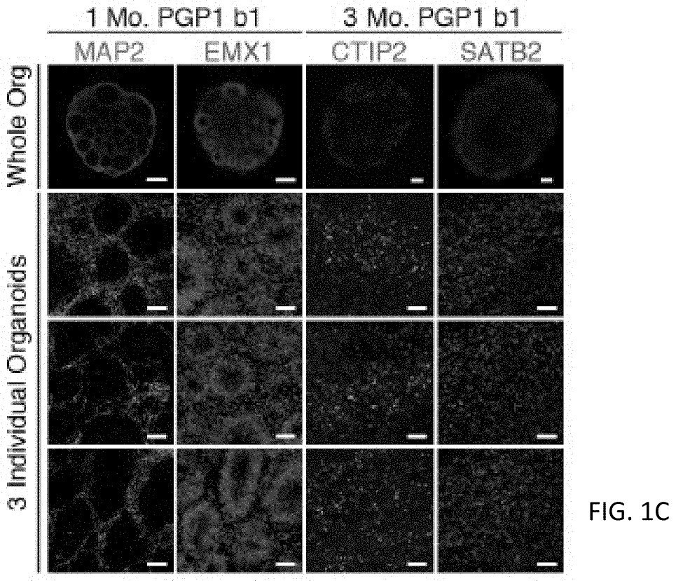

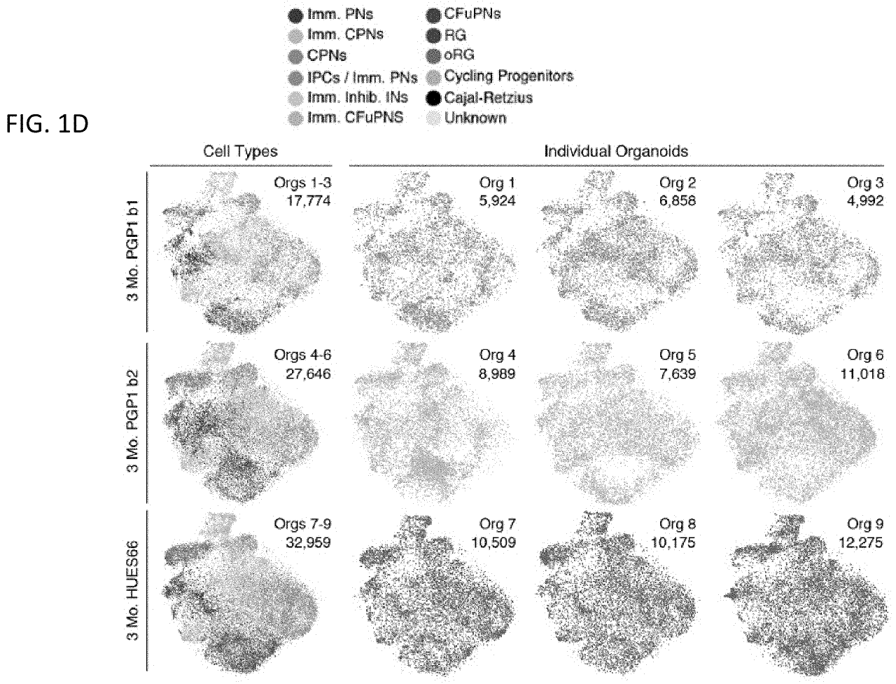

To standardize and compare these four models, cultures of each were derived from the same hiPSC line, PGP1¹⁶. Substantial differences in overall external morphology and size across models (FIG. 5A) were observed. Self-patterned whole-brain organoids showed the greatest variability in shape. The two patterned spheroid models showed more consistent shape but remained substantially smaller. The dorsally patterned organoid model, however, showed desirable features of large size and consistent overall shape (FIGS. 1A-1B and FIG. 5A). For this model, the protocol originally published by Kadoshima et al.⁵ was modified to eliminate the need for interventions to reduce hypoxia by adapting the cultures to growth in spinner-flask bioreactors (FIG. 1A), allowing substantially extended development in vitro (FIG. 5A). Immunohistochemistry (IHC) for the dorsal forebrain progenitor markers EMX1 and PAX6 and for the early pan-neuronal marker MAP2 confirmed the presence of rosette-like structures at one month, when dorsalized progenitors lined ventricle-like cavities. The cortical pyramidal neuron subtype markers CTIP2 and SATB2 were expressed by 3 months and subsequently maintained (FIG. 1C and FIG. 5B). Importantly, these features were observed in the majority of organoids across 5 distinct stem cell lines: PGP1 (male, hiPSC; three independent experimental batches), HUES66 (female, hESC; two independent batches), GM08330 (male, hiPSC), 11a (male, hiPSC), and Mito 210 (male, hiPSC). Across all lines, 100% of organoids expressed PAX6 and MAP2 at 1, 3, and 6 months, and 89% also expressed EMX1 (Extended Data Table 1). Given these promising features, further analysis was concentrated on this model.

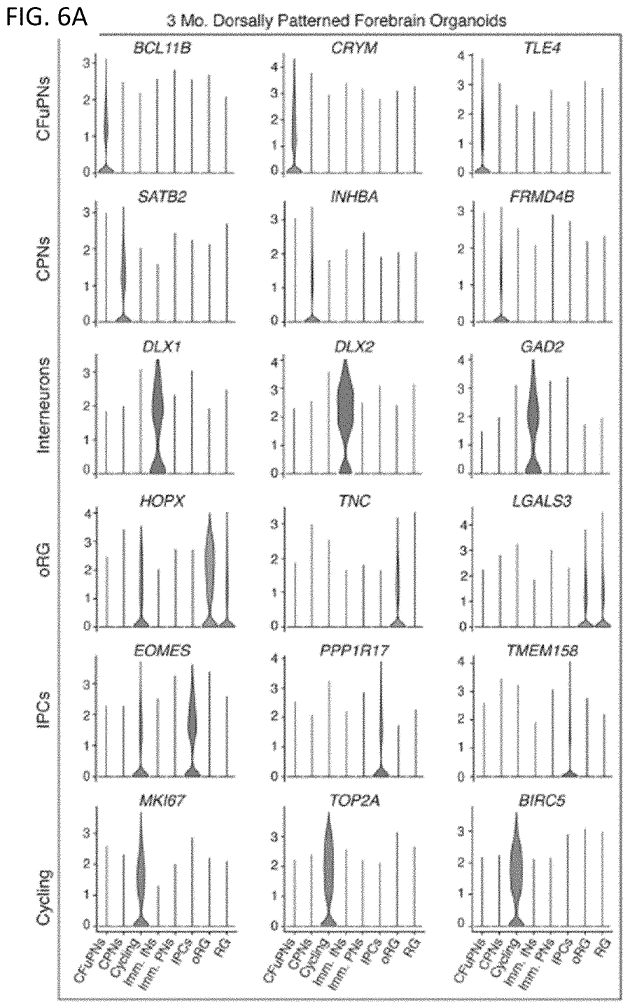

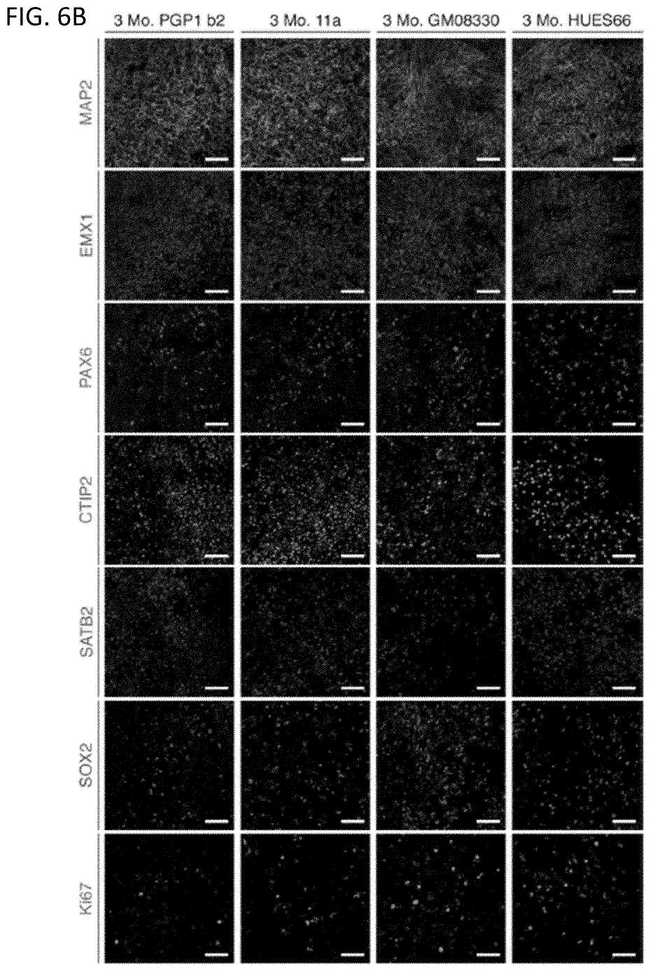

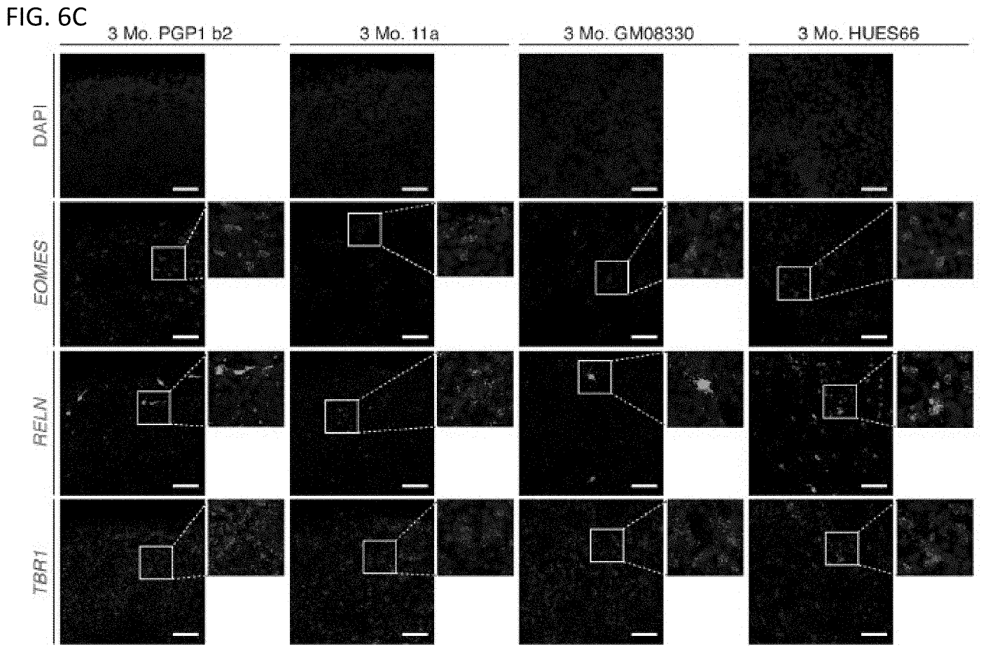

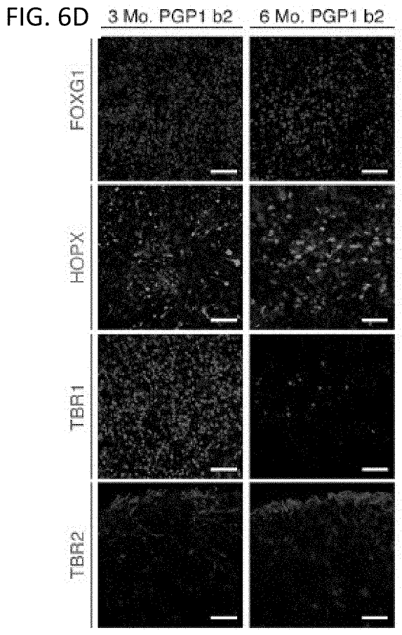

High-throughput single cell RNA-seq (scRNA-seq) was performed on 78,379 cells from 9 individual organoids from two stem cell lines, PGP1 (two independent batches, b1 and b2) and HUES66 (one batch), at 3 months of growth (FIG. 1D). For each batch, cells from all organoids were clustered and systematically classified by comparing signatures of differentially expressed genes (Extended data Table 1) to pre-existing datasets of endogenous cell types^{3,8,17-23} (examples in FIG. 6A). This defined eleven main transcriptionally-distinct cell types across both lines, representing a large diversity of progenitor and neuronal types appropriate for the cerebral cortex (FIG. 1D).

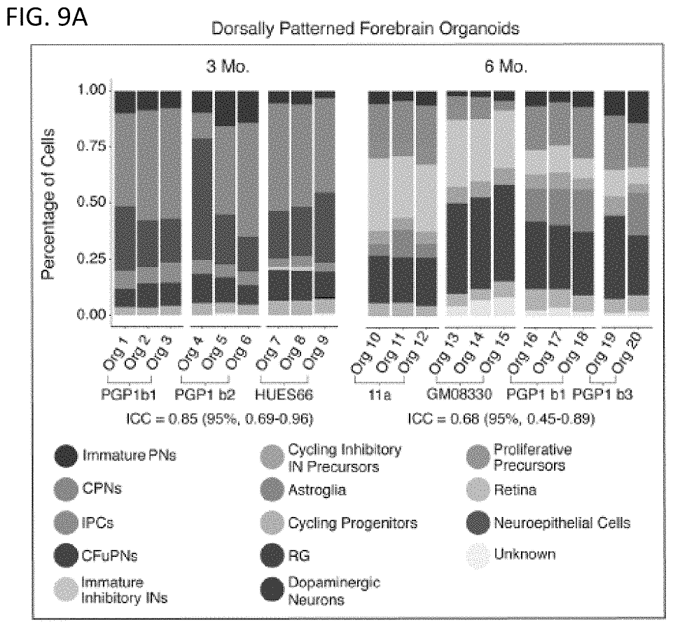

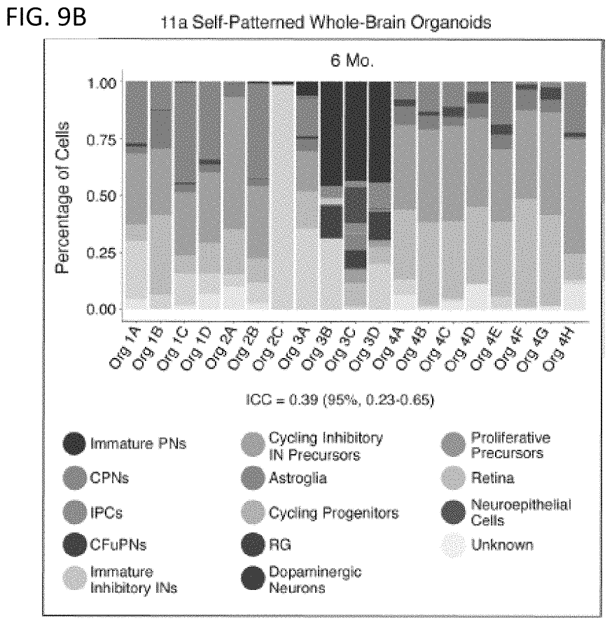

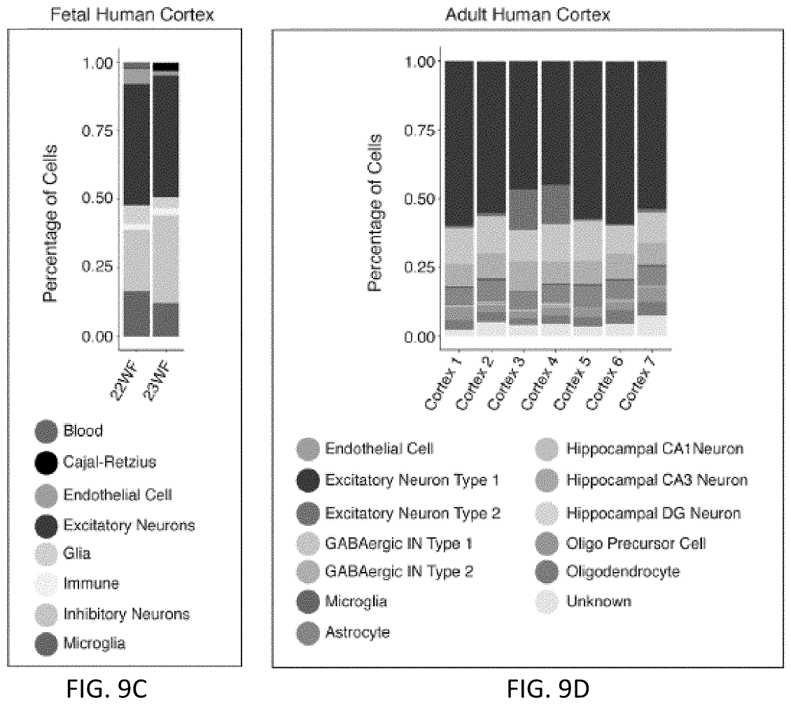

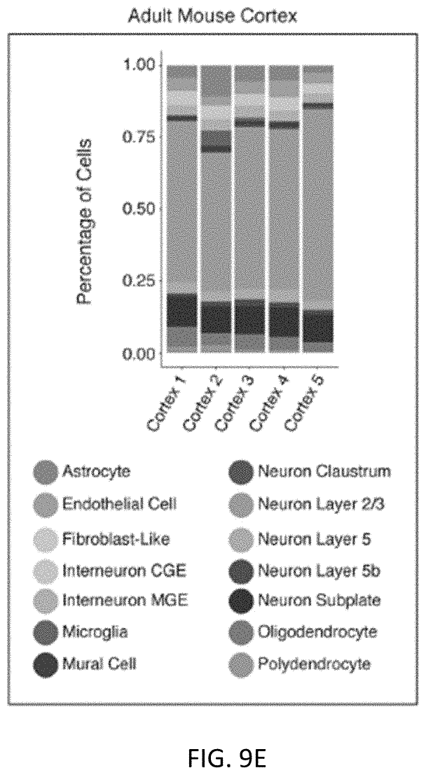

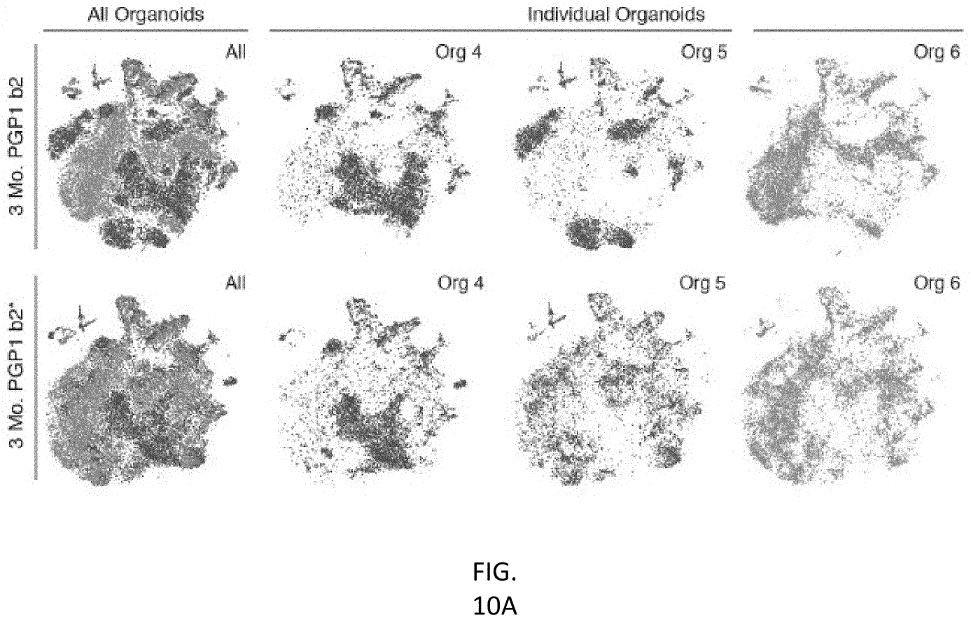

To determine whether each organoid had generated the complete set of cells, the cells from all 9 organoids were aligned and co-clustered (FIG. 1D). Organoids were highly reproducible in cellular composition across different lines and batches (FIG. 1D). Furthermore, the cell type assignments from the batch-by-batch analysis were grouped together by the co-clustering analysis, indicating consistent transcriptional signatures for individual cell types across lines and batches. Although one organoid (Org 4) had an increased number of corticofugal projection neurons, the overall proportions of individual cell types were consistent across cell lines (see also FIG. 9A). Expression of cell type-specific markers assayed by IHC and RNA in situ hybridization showed equally high consistency (FIGS. 6B-6D). These organoids not only produce a large variety of cortical cell types, but cell identity and diversity are reproducible organoid-to-organoid and across experiments.

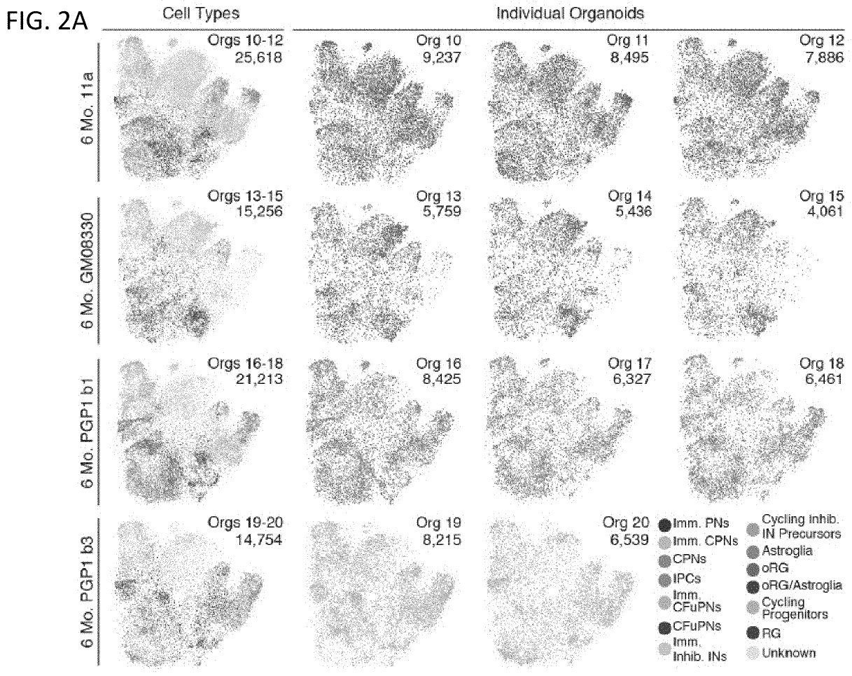

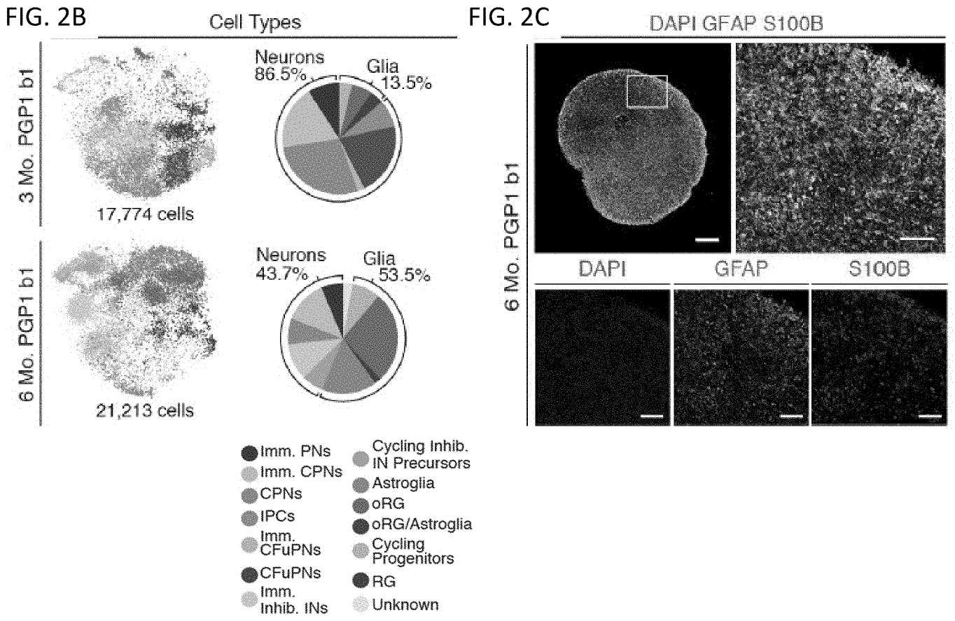

Cellular diversity of the cerebral cortex develops in a specific temporal sequence^{23,24}. Astroglial cells, which are typically produced later in development, were notably under-represented in the 3-month cultures. Therefore, organoids from the PGP1, GM08330, and 11a stem cell lines were grown for six months and scRNA-seq of an additional 87,863 cells from 12 individual organoids (PGP1: two independent batches, b1 and b3; GM08330 and 11a: one batch) (FIG. 2A) was performed. One out of the 12 organoids did not show expression of FOXG1 or any other neuronal marker, suggesting that differentiation had stalled at an early stage. Therefore, we excluded this organoid from further analysis. Co-clustering of all cells in the remaining 11 organoids identified thirteen main cell classes (FIG. 2A). Importantly, organoid-to-organoid variability remained extremely low even after 6 months of culture.

To quantify changes in cellular composition over time, cells from organoids derived from the same differentiation batch (PGP1 b1) but collected at 3 and 6 months (FIG. 2B) were combined and co-clustered. The results are shown in the following table:

| | 3 Months PGP1 batch 1 | | | | |
|---|---|---|---|---|---|
| | Org 1 | Org 2 | Org 3 | Percentage | % |
| Astroglia | 0 | 0 | 0 | 0 | 0 |
| CFuPNs | 1316 | 971 | 625 | 0.16383481 | 16.4 |
| CPNs | 1524 | 2001 | 1424 | 0.27844042 | 27.8 |
| Cycling | 222 | 240 | 201 | 0.03730168 | 3.7 |
| Immature CFuPNs | 394 | 463 | 342 | 0.06745808 | 6.7 |
| Immature CPNs | 921 | 1354 | 1033 | 0.18611455 | 18.6 |
| Immature | 0 | 0 | 0 | 0 | 0 |
| Interneurons | | | | | |
| Immature PNs | 597 | 605 | 396 | 0.08990661 | 9.0 |
| IPCs | 471 | 488 | 444 | 0.07893552 | 7.9 |
| oRG | 319 | 420 | 317 | 0.05941263 | 5.9 |
| RG | 160 | 316 | 210 | 0.0385957 | 3.9 |
| Cycling IN Precursors | 0 | 0 | 0 | 0 | 0 |

| | 6 Months PGP1 batch 1 | | | | |
|---|---|---|---|---|---|
| | Org 16 | Org 17 | Org 18 | Percentage | % |
| Astroglia | 1226 | 1001 | 1211 | 0.16685271 | 16.7 |
| CFuPNs | 0 | 0 | 0 | 0 | 0 |
| CPNs | 534 | 417 | 511 | 0.07095365 | 7.1 |
| Cycling | 787 | 505 | 458 | 0.08493084 | 8.5 |
| Immature CFuPNs | 0 | 0 | 0 | 0 | 0 |
| Immature CPNs | 1033 | 739 | 910 | 0.13016258 | 13.0 |
| Immature | 923 | 768 | 570 | 0.10973065 | 11.0 |
| Interneurons | | | | | |
| Immature PNs | 573 | 321 | 468 | 0.06610046 | 6.6 |
| IPCs | 71 | 56 | 43 | 0.00825042 | 0.8 |
| oRG | 2331 | 1692 | 1710 | 0.27823344 | 27.8 |
| RG | 199 | 113 | 106 | 0.02028634 | 2.0 |
| Cycling IN Precursors | 531 | 463 | 335 | 0.06449891 | 6.4 |

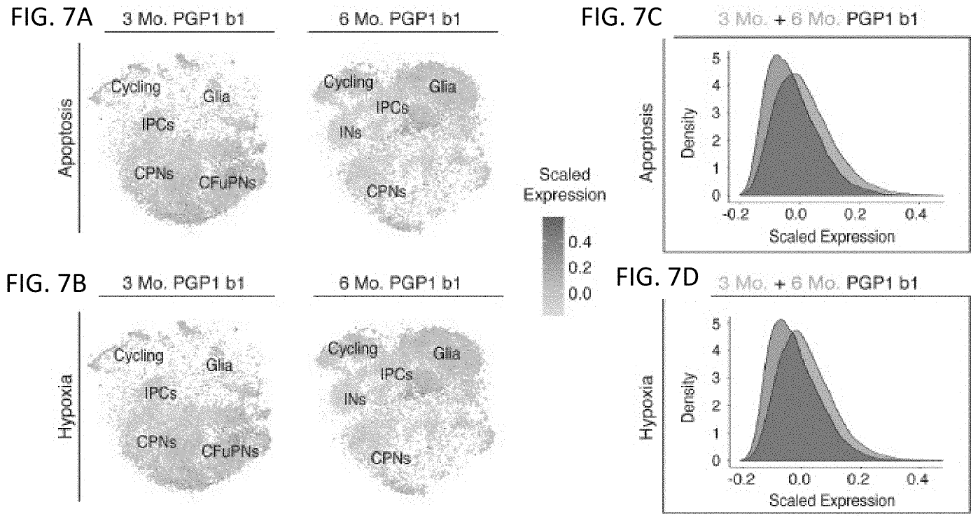

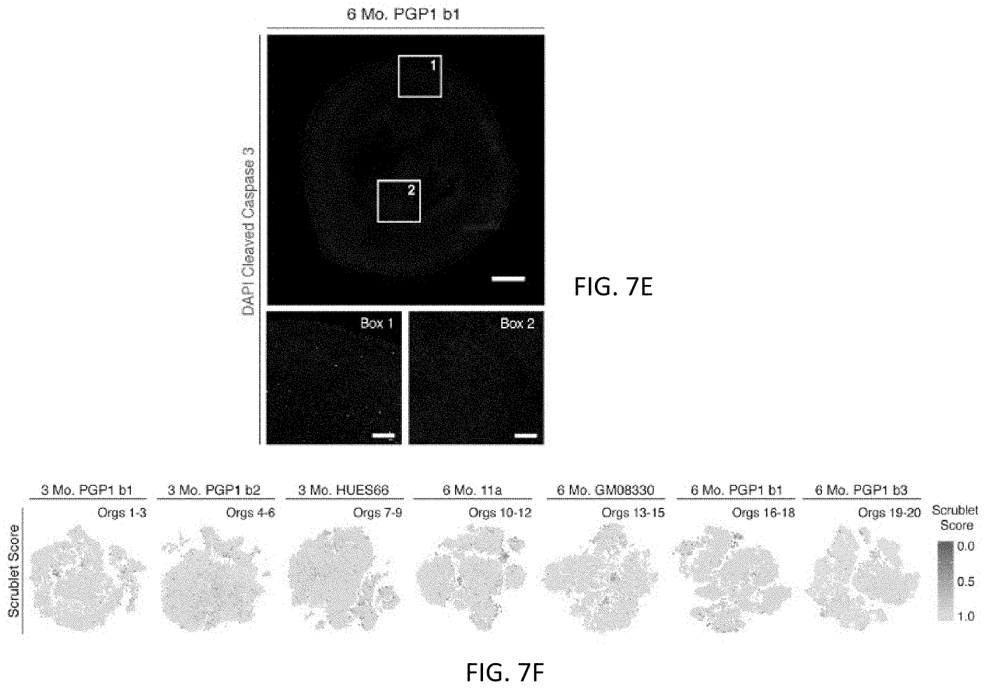

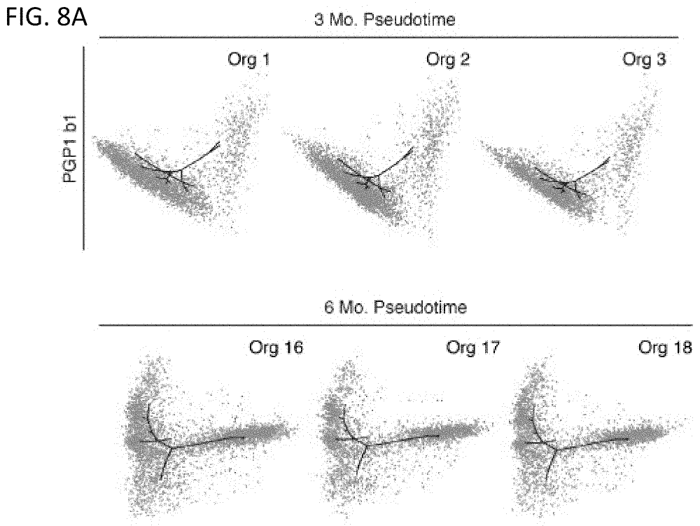

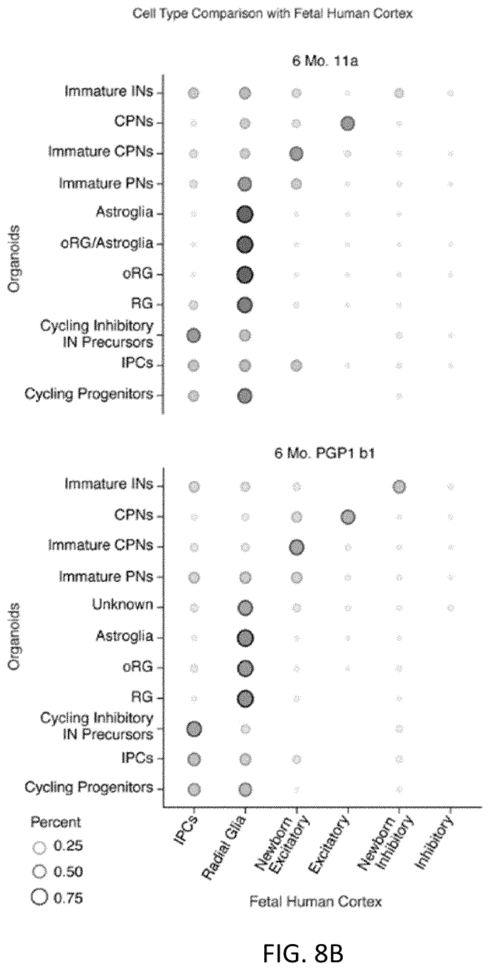

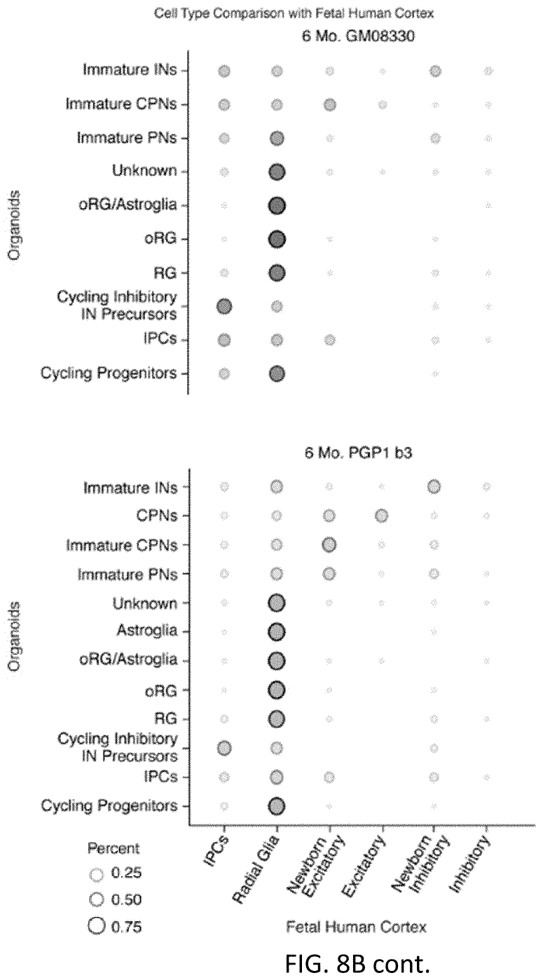

The six-month cultures contained substantial numbers of astrocytes, confirmed by IHC for the astrocytic markers S100B and GFAP (FIG. 2C), as well as cycling inhibitory interneuron precursors and immature inhibitory interneurons (FIGS. 2A-2B). While the origin of these interneurons is unclear, their transcriptional similarity to IPCs (see also FIG. 8B) suggests that they could be interneurons of the olfactory bulb, which originate from subventricular zone progenitors²⁵. The data indicate that appropriate additional cell types develop in these organoids, respecting endogenous temporal sequences. Despite the long culture period, molecular markers of apoptosis and hypoxia remained low (FIGS. 7A-7E).

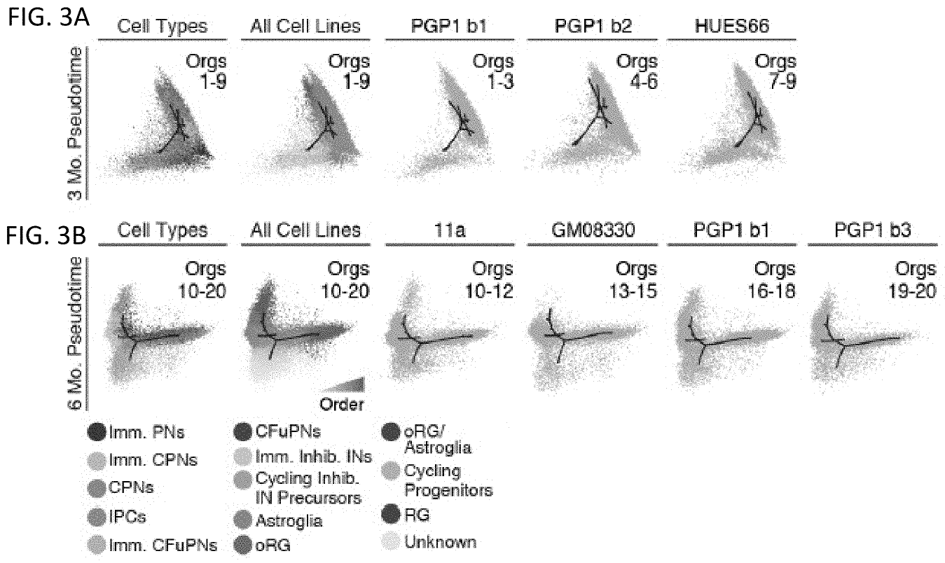

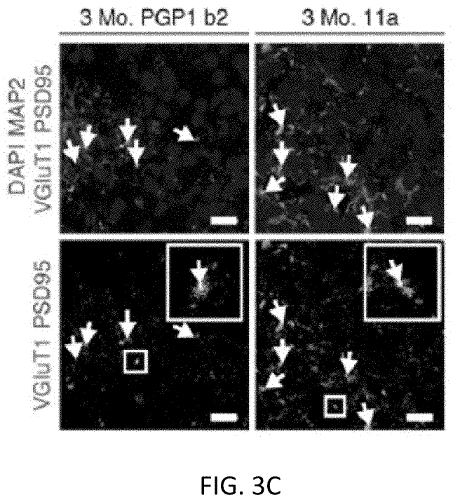

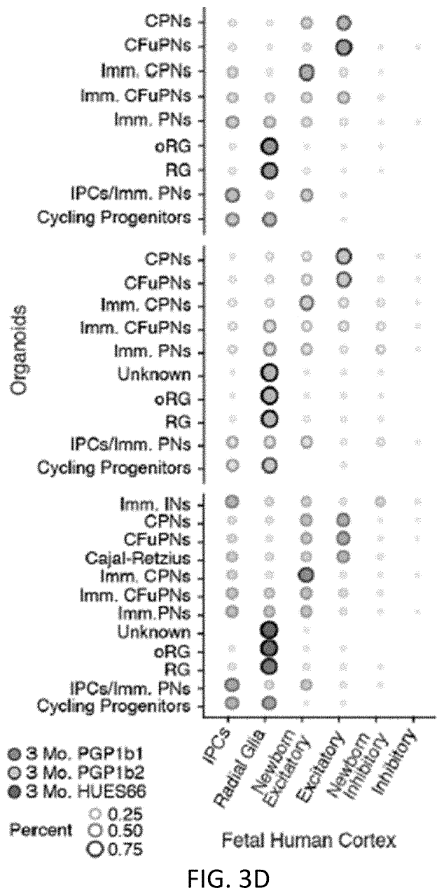

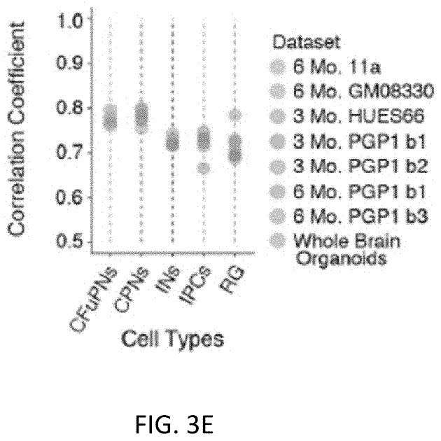

It is possible that, within individual organoids, the same terminal composition of cell types may be generated via distinct developmental trajectories. Therefore, a developmental pseudotime trajectory²⁶ across all cell lines for each timepoint was calculated. Each organoid distributed similarly along the pseudotime trajectory, independent of line or batch, and the pseudotemporal ordering of cell types approximated that of in vivo human development (FIGS. 3A-3B and FIG. 8A). At three months, all organoids followed a trajectory that progressed from radial glia to mature excitatory neurons. Confirming neuronal maturation, colocalization of the pre- and post-synaptic markers VGluT1 and PSD95 suggested the presence of excitatory synapses at this timepoint (FIG. 3C). At six months, the trajectory proceeded along two branches, towards excitatory neurons or mature astrocytes. The data indicate that beyond terminal cellular composition, developmental trajectories of fate specification are reproducibly established in each organoid and therefore can be reliably investigated in this system.

To assess similarity between organoid cell types and those of the endogenous human brain, a published scRNA-seq dataset of human fetal cerebral cortex¹⁸ was employed. Briefly, fetal human cells were used to train a Random Forest classifier, which was then applied to assign organoid cells to the human cortex cell categories²⁷. Organoid cells at both 3 and 6 months were predominantly assigned to the corresponding endogenous cell class, indicating that their transcriptional profiles were similar to endogenous cells (FIG. 3D and FIG. 8B). Importantly, the main cell types present in organoids at 3 months are as similar to the corresponding endogenous cells as those present at 6 months, suggesting that by 3 months organoids are already a valuable tool for modeling human neurodevelopmental processes.

It is unclear whether treatment with exogenous patterning signals may negatively affect the fidelity of terminal cell identities. Therefore, whether transcriptional similarity to endogenous human fetal cortex¹⁸ cells was comparable to that found in self-patterned whole-brain organoids analyzed in a previous study³ (see Methods) was investigated. All of the dorsally patterned organoids and the whole-brain organoids showed similarly high correlation with human cells (correlation coefficients between 0.67 and 0.80; FIG. 3E). Therefore, the use of exogenous patterning signals in this model does not require a trade-off in the fidelity of the cell types generated.

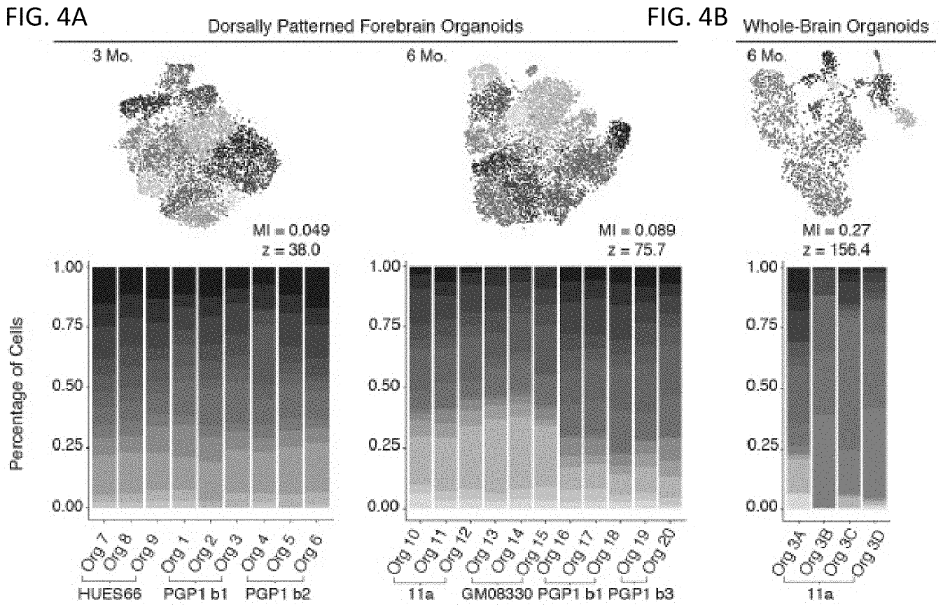

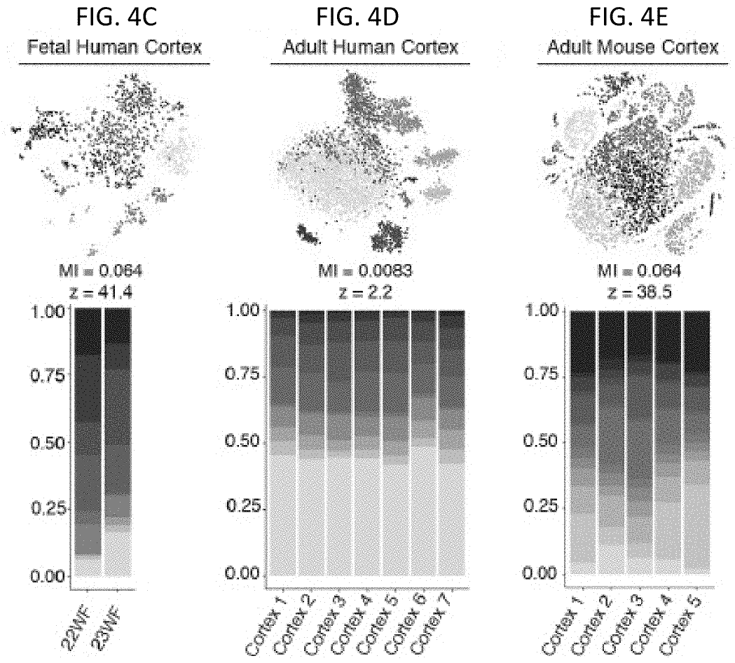

To evaluate the reproducibility of this model relative to prior systems, the variability of dorsally patterned forebrain organoids and self-patterned whole-brain organoids³ (FIGS. 4A-4B and FIGS. 9A-9B) was compared. scRNA-seq data for the 19 individual self-patterned whole-brain organoids in a previous study³ showed high organoid-to-organoid variability, with only a minority of organoids (4/19) generating substantial amounts of forebrain cells (FIG. 9B). Intraclass correlation (ICC), a correlation metric that considers group structure in the data (i.e., cell types), was applied to the proportions of each cell type produced by each organoid; an ICC near 1.0 indicates high agreement. The 19 organoids in the whole-brain organoid cohort had an ICC of 0.39 (95% Confidence Interval [CI] 0.23-0.65). In contrast, the pre-patterned organoids of the present study scored much higher (3 month organoids: ICC 0.85, 95% CI: 0.69-0.96; 6 month organoids: ICC 0.68, 95% CI: 0.45-0.89) (FIG. 9A).

The results of analysis of cell types for Organoids 1-3 and 5-10 cultured for three months (Organoid 4 is omitted as containing large deviations in CFuPNs and CPNs as compared to the other 9 organoids) are:

| | | Standard |
|---|---|---|
| 3 Months | Average | Dev |
| CFuPNs | 0.22459765 | 0.05520868 |
| CPNs | 0.4513659 | 0.04508055 |
| Cajal-Retzius | 0.00236614 | 0.00260036 |
| Cycling | 0.056674 | 0.01195368 |
| IPCs | 0.04749755 | 0.01034576 |
| Immature | 0.00682442 | 0.00905674 |
| Interneurons | | |
| Immature PNs | 0.09087921 | 0.05637404 |
| RG | 0.11590331 | 0.01727356 |
| Unknown | 0.00389184 | 0.00449002 |

The results of analysis of cell types for Organoids 11-20 cultured for 6 months are:

| | | Standard |
|---|---|---|
| 6 months | Average | Dev |
| Astroglia | 0.08407071 | 0.07776454 |
| CPNs | 0.14556994 | 0.07029864 |
| Cycling | 0.06502939 | 0.01377207 |
| IPCs | 0.04044299 | 0.02713488 |
| Immature | 0.20321577 | 0.09919859 |
| Interneurons | | |
| Immature PNs | 0.06434523 | 0.0349891 |
| RG | 0.30597755 | 0.0832174 |
| Unknown | 0.02879936 | 0.02784556 |
| Ventral Precursors | 0.06254906 | 0.01303484 |

The availability of single-cell RNA-seq datasets from individual human^{28,29} and mouse³⁰ cortices allowed investigation of if the reproducibility of cell types generated in dorsally patterned organoids was similar to that of endogenous brains. As existing datasets vary in the terminology and degree of granularity used for cell type assignment (FIG. 9), each dataset was first re-clustered using identical methods. Mutual information (MI) scores were calculated between cluster assignments and sample identity in each dataset (FIGS. 4A-4E); a lower MI score and lower z-score indicate higher reproducibility between individuals. For the self-patterned whole-brain organoid model³, the best performing batch of 4 organoids that produced forebrain cells were specifically examined; these organoids had a high MI score of 0.27 and a z-score of 156.4 (FIG. 4B). Conversely, in the dorsally patterned organoids, reproducibility was consistently high, as reflected in low MI and z-scores (MI 0.049-0.089, z-score 38.0-75.7) (FIG. 4A). Remarkably, the reproducibility with which the different clusters are generated in individual dorsally patterned organoids (FIG. 4A and FIG. 9A) is comparable to that of individual endogenous human or mouse brain samples (MI range 0.008-0.064, z-score range 2.2-41.4) (FIGS. 4C-4E and FIGS. 9C-9E). All datasets, including the human and mouse endogenous brains, showed variation between samples; however, the degree of variation in the dorsally patterned organoid samples was similar to that of the endogenous brain datasets.

Stem cell-based models of the human brain offer an opportunity to investigate processes of human brain development and disease that would not otherwise be experimentally accessible. However, their application has been limited by a variety of factors, prime among which is the lack of developmental reproducibility between individual organoids. Here, through the largest-to-date scRNA-seq dataset in brain organoids, it is demonstrated that extensive cellular diversity of a complex CNS region can be generated outside of the embryo in a highly reproducible and developmentally-constrained manner that transcends individual organoids, experimental batch, genetic background, and sex. These data suggest that dynamic processes of development can be modeled in organoids, and that early patterning of a brain region starts a process of cell specification and maturation that is highly constrained even outside the embryo. This level of reproducibility in vitro informs about basic constraints and principles that control cell diversification in the human brain, and establishes a valuable organoid model amenable to the experimental investigation of developmental abnormalities associated with human neurological disease that would otherwise not be experimentally accessible.

EXTENDED DATA TABLE 1

| | | | Expression of Neuronal and Cortical Markers by IHC | | | | | | |
|---|---|---|---|---|---|---|---|---|---|
| | | | Organoids | | | | | | |
| Timepoint | Cell Line | Batches | Analyzed | MAP2 | | PAX6 | | EMX1 | |
| 1 Mo. | PGP1 | 2 | 7 | 7/7 | 100% | 7/7 | 100% | 7/7 | 100% |
| | 11a | 1 | 4 | 4/4 | 100% | 4/4 | 100% | 4/4 | 100% |
| | GM08830 | 1 | 6 | 6/6 | 100% | 6/6 | 100% | 6/6 | 100% |
| | Mito 210 | 1 | 4 | 4/4 | 100% | 4/4 | 100% | 4/4 | 100% |
| | HUES66 | 2 | 5 | 5/5 | 100% | 5/5 | 100% | 5/5 | 100% |
| | Total | 7 | 26 | 26/26 | 100% | 26/26 | 100% | 26/26 | 100% |
| 3 Mo. | PGP1 | 3 | 10 | 10/10 | 100% | 10/10 | 100% | 9/10 | 90% |
| | 11a | 1 | 6 | 6/6 | 100% | 6/6 | 100% | 6/6 | 100% |
| | GM08330 | 1 | 6 | 6/6 | 100% | 6/6 | 100% | 6/6 | 100% |
| | Mito 210 | 1 | 3 | 3/3 | 100% | 3/3 | 100% | 3/3 | 100% |
| | HUES66 | 2 | 12 | 12/12 | 100% | 12/12 | 100% | 12/12 | 100% |
| | Total | 8 | 37 | 37/37 | 100% | 37/37 | 100% | 37/37 | 100% |
| 6 Mo. | PGP1 | 2 | 6 | 6/6 | 100% | 6/6 | 100% | 5/6 | 83% |
| | 11a | 1 | 3 | 3/3 | 100% | 3/3 | 100% | 3/3 | 100% |
| | GM08330 | 1 | 3 | 3/3 | 100% | 3/3 | 100% | 3/3 | 100% |
| | HUES66 | 1 | 7 | 7/7 | 100% | 7/7 | 100% | 0/7 | 0% |
| | Total | 5 | 19 | 19/19 | 100% | 19/19 | 100% | 11/19 | 58% |

| | | | | | | | | |
|---|---|---|---|---|---|---|---|---|
| Total | 8 | 82 | 82/82 | 100% | 82/82 | 100% | 73/82 | 89% |

| | | | Generation of Forebrain | | |
|---|---|---|---|---|---|
| | | | Cell Types by RNA-seq | | |
| | | | Organoids | | |
| Timepoint | Cell Line | Batches | Analyzed | Reproducibility | |
| 1 Mo. | PGP1 | 2 | | | |
| | 11a | 1 | | | |
| | GM08830 | 1 | | | |
| | Mito 210 | 1 | | | |
| | HUES66 | 2 | | | |
| | Total | 7 | | | |
| 3 Mo. | PGP1 | 3 | 6 (2 batches) | 6/6 | 100% |
| | 11a | 1 | | | |
| | GM08330 | 1 | | | |
| | Mito 210 | 1 | | | |
| | HUES66 | 2 | 3 | 3/3 | 100% |
| | Total | 8 | 9 | 9/9 | 100% |
| 6 Mo. | PGP1 | 2 | 6 (2 batches) | 5/6 | 83% |
| | 11a | 1 | 3 | 3/3 | 100% |
| | GM08330 | 1 | 3 | 3/3 | 100% |
| | HUES66 | 1 | | | |
| | Total | 5 | 12 | 11/12 | 92% |

| | | | | |
|---|---|---|---|---|
| Total | 8 | 21 | 20/21 | 95% |

Extended Data Table 1. Efficiency of dorsal forebrain cell type generation as assessed by IHC for neuronal (MAP2) and dorsal progenitor (PAX6 and EMX1) markers at 1, 3 and 6 months, and by single-cell RNA-seq analysis at 3 and 6 months. In addition to data for the 6 experimental batches described in this study, data for two additional batches, a replicate for the HUES66 and one batch for the additional line Mito 210, are also included in the table. Detailed information about repetitions and efficiency can be found in Methods, in the “Statistics and Reproducibility” section.

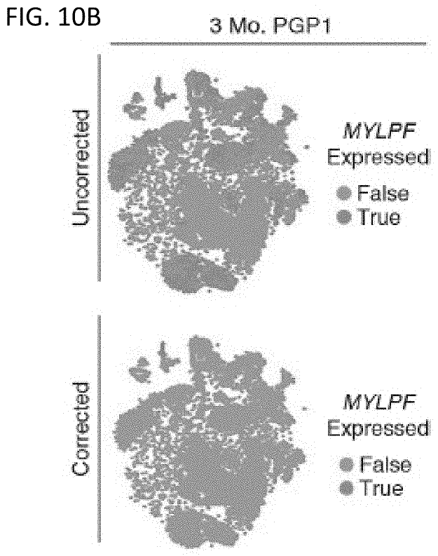

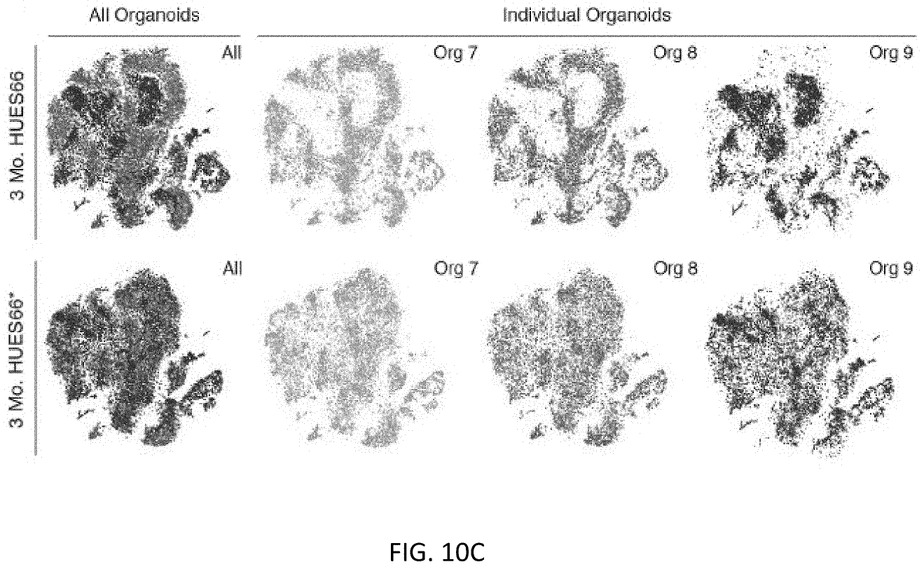

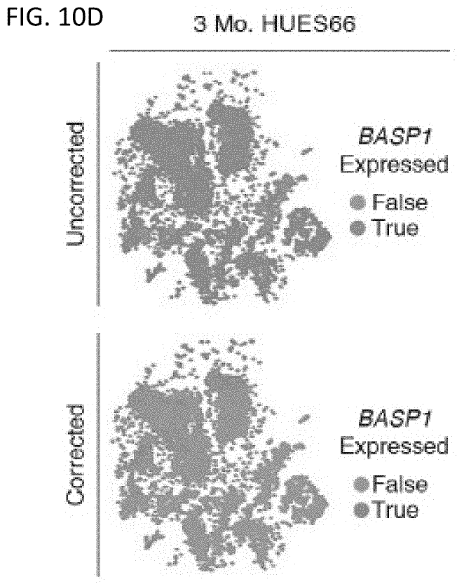

Batch-corrected data from all of the timepoint-matched organoids was provided together in FIGS. 1 and 2. Before batch correction, in two cases there were single organoids that, while making all of the same cell types, displayed a distinct cluster pattern (FIGS. 10A and 10C, top rows). It was determined that the distinct cluster pattern did not in fact reflect a biological difference, but was due to ambient RNA contamination, a common source of noise in single-cell RNA-seq experiments. This effect was evaluated using the SoupX R package v0.3.0, which uses “empty” droplets in the single-cell RNA-seq dataset (nUMI<10) to identify which mRNAs are present in the ambient media (Young & Behjati, “SoupX removes ambient RNA contamination from droplet based single-cell RNA sequencing data”, bioRxiv 303727). Default parameters were used. Several genes were found to significantly contribute to ambient RNA in the lanes containing the affected organoids (Org 5 and Org 9). To correct for this, many of these genes were removed from the list of highly variable genes used for clustering in that batch. For PGP1, these genes were mainly related to mesodermal functions, and were MYLPF, ACTC1, TNNC2, TNNI1, TNNC1, MEF2C, TPM2, ACTA1, MYL4, DES, MYL1, TNNI2, MYH3, TNNT3, and ENO3. For HUES66, these genes were H1FX, BASP1, GFBP2, RNF187, UBE2S, TCEAL5, FJX1, SRM, SMS, IER5, ID4, DUSP5, SFRP1, RPRM, MEDI, YBX3, and KCNG1.

After correcting for ambient RNA, the organoids from these batches overlap closely (FIGS. 10A and 10C, bottom row). Note that this does not mask all clustering differences, such as the increased number of corticofugal projection neurons found in Org 4 (bottom of purple t-SNE in FIG. 10A). The expression of representative genes that were found to contribute to organoid-specific gene expression differences is shown before and after ambient RNA correction in FIGS. 10B and 10D (one out of 15 genes for PGP1 b2, one out of 17 genes for HUES66). While this demonstrates where the source of variation originated, the original data for all organoids (rather than use the SoupX corrected data) was retained to avoid any possibility of distortion from the ambient mRNA correction algorithm.

EXTENDED DATA TABLE 4

| | | Expression of Neuronal and Cortical Markers by IHC | | | | | | | |
|---|---|---|---|---|---|---|---|---|---|
| | | | Organoids | | | | | | |
| Timepoint | Cell Line | Batches | Analyzed | MAP2 | | PAX6 | | EMX1/FOXG1 | |
| 1 Mo. | 11a | 2 | 5 | 5/5 | 100% | 5/5 | 100% | 5/5 | 100% |
| | GM08330 | 1 | | | | | | | |
| | Mito 210 | 3 | 12 | 12/12 | 100% | 8/8 | 100% | 12/12 | 100% |
| | HUES66 | 1 | 2 | 2/2 | 100% | 2/2 | 100% | 2/2 | 100% |
| | Total | 7 | 19 | 19/19 | 100% | 15/15 | 100% | 19/19 | 100% |
| 3 Mo. | 11a | 1 | 3 | 3/3 | 100% | 3/3 | 100% | 3/3 | 100% |
| | GM08330 | 1 | 2 | 2/2 | 100% | | | 2/2 | 100% |
| | Mito 210 | 1 | 6 | 6/6 | 100% | 6/6 | 100% | 6/6 | 100% |
| | HUES66 | 1 | | | | | | | |
| | Total | 4 | 11 | 11/11 | 100% | 9/9 | 100% | 11/11 | 100% |
| 6 Mo. | Mito 210 | 1 | 2 | 2/2 | 100% | 2/2 | 100% | 2/2 | 100% |
| | Total | 1 | 2 | 2/2 | 100% | 2/2 | 100% | 2/2 | 100% |

| | | | | | | | | |
|---|---|---|---|---|---|---|---|---|
| Total | 12 | 32 | 32/32 | 100% | 32/32 | 100% | 32/32 | 100% |

| | | | Generation of Forebrain | | |
|---|---|---|---|---|---|
| | | | Cell Types by RNA-seq | | |
| | | | Organoids | | |
| Timepoint | Cell Line | Batches | Analyzed | Reproducibility | |
| 1 Mo. | 11a | 2 | | | |
| | GM08330 | 1 | 3 | 3/3 | 100% |
| | Mita 210 | 3 | 9 (3 batches) | 9/9 | 100% |
| | HUES66 | 1 | | | |
| | Total | 7 | 12 | 12/12 | 100% |
| 3 Mo. | 11a | 1 | | | |
| | GM08830 | 1 | 3 | 3/3 | 100% |
| | Mito 210 | 1 | 3 | 3/3 | 100% |
| | HUES66 | 1 | 3 | 3/3 | 100% |
| | Total | 4 | 9 | 9/9 | 100% |
| 6 Mo. | Mito 210 | 1 | | | |
| | Total | 1 | | | |

| | | | | |
|---|---|---|---|---|
| Total | 12 | 21 | 21/21 | 100% |

Extended Data Table 4. Efficiency of dorsal forebrain cell type generation as assessed by IHC for neuronal (MAP2) and dorsal progenitor (PAX6 and EMX1 or FOXG1) markers and by single-cell RNA-seq analysis at 1, 3 and 6 months of additional experimental batches: two replicates of the 11a, one replicate of the GM08330, one replicate of the HUES66, and 3 batches for the additional line Mito 210.

In addition to data for the experimental batches described herein, additional replicates of the 11a, GM8330, HUES66, and Mito 210 were analyzed.

A total of 32 additional single organoids collected at 1, 3 and 6 months, derived from 4 different cell lines, from 7 independent batches (11a: 2 batches; GM08330 and HUES66: 1 batch each; and Mito 210: 3 batches) were analyzed by IHC for expression of MAP2, PAX6, and EMX1 or FOXG1. All organoids tested were positive at each time point: at 1 month (n=19/19 positive for MAP2 and EMX1/FOXG1; and 15/15 positive for PAX6); 3 months (n=11/11 positive for MAP2 and EMX1/FOXG1; and 9/9 positive for PAX6); and 6 months (n=2/2 positive) (100% efficiency) (Extended Data Table 4).

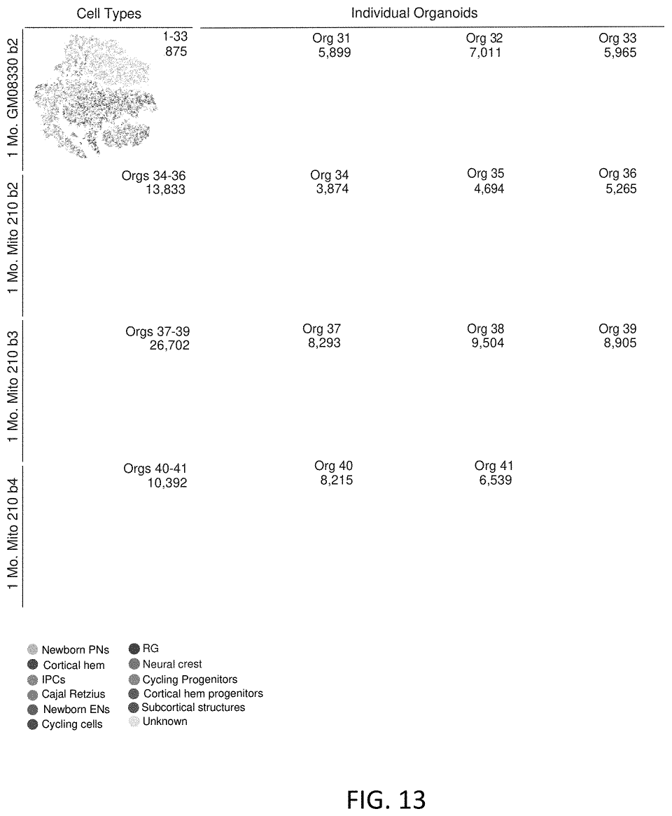

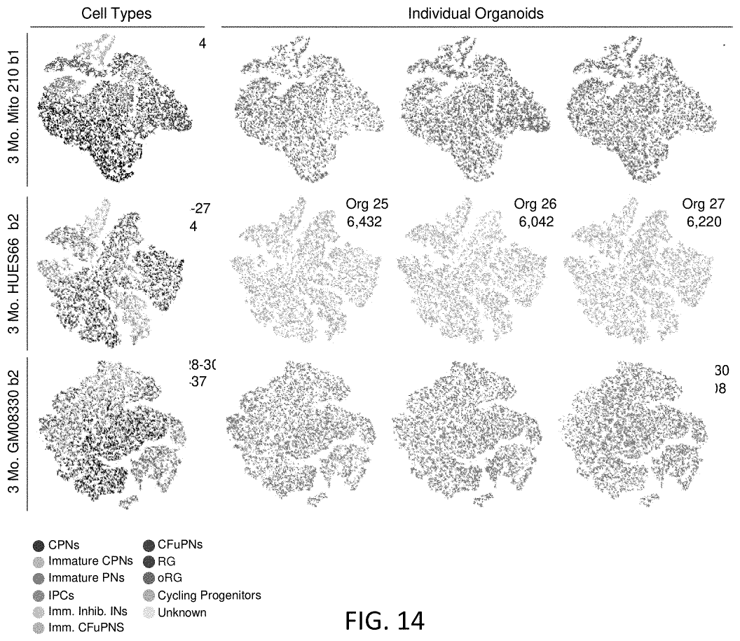

A total of 21 additional single organoids collected at 1 and 3 months, derived from 3 different cell lines, from 5 independent batches (GM08330: 1 batch; Mito 210: 3 batches; and HUES66: 1 batch; n=3 organoids per batch and timepoint) were profiled by single-cell RNA-seq. Analysis of 133,897 single cells revealed reproducibility in the diversity of cell types generated “organoid-to-organoid” for 21/21 organoids (100% efficiency) (Extended Data Table 4 and FIGS. 13-14).

Methods

Pluripotent stem cell (PSC) culture. The PGP1 (Personal Genome Project 1) hiPSC line was from the lab of George Church¹⁶; the HUES66 hESC and the 11a hiPSC lines from the Harvard Stem Cell Institute; the GM08330 hiPSC line³¹ (also known as GM8330-8)³² from the lab of Michael Talkowski (MGH Hospital); and the Mito 210 hiPSC line from the lab of Bruce Cohen (McLean Hospital). All PSC lines were cultured in feeder-free conditions on Geltrex (Gibco)-coated cell culture dishes, using mTESR1 medium (Stem Cell Technologies) with 100 U/mL penicillin and 100m/mL streptomycin (Corning), at 37° C. in 5% CO₂. All human PSCs were maintained below passage 50, were negative for mycoplasma (assayed with the MycoAlert™ PLUS Mycoplasma Detection Kit, Lonza), and karyotypically normal (G-banded Karyotype test, performed by WiCell Research Institute, Inc.).

Organoid differentiation. To generate dorsally patterned forebrain organoids, we modified the method previously described in Kadoshima et al.³. We eliminated the need for growth under 40% O₂, the need for cell aggregates to be periodically bisected, and the use of high O₂ penetration dishes, by adapting the cultures to growth in spinner-flask bioreactors. Specifically, on day 0, feeder-free cultured human PSCs, 80-90% confluent, were dissociated to single cells with Accutase (Gibco), and 9,000 cells per well were reaggregated in ultra-low cell-adhesion 96-well plates with V-bottomed conical wells (sBio PrimeSurface plate; Sumitomo Bakelite) in Cortical Differentiation Medium (CDM) I, containing Glasgow-MEM (Gibco), 20% Knockout Serum Replacement (Gibco), 0.1 mM Minimum Essential Medium non-essential amino acids (MEM-NEAA) (Gibco), 1 mM pyruvate (Gibco), 0.1 mM 2-mercaptoethanol (Gibco), 100 U/mL penicillin, and 100m/mL streptomycin (Corning). From day 0 to day 6, ROCK inhibitor Y-27632 (Millipore) was added to the medium at a final concentration of 20 μM. From day 0 to day 18, Wnt inhibitor IWR1 (Calbiochem) and TGFβ inhibitor SB431542 (Stem Cell Technologies) were added at a concentration of 3 μM and 5 μM, respectively. From day 18, the floating aggregates were cultured in ultra-low attachment culture dishes (Corning) under orbital agitation (70 rpm) in CDM II, containing DMEM/F12 medium (Gibco), 2 mM Glutamax (Gibco), 1% N2 (Gibco), 1% Chemically Defined Lipid Concentrate (Gibco), 0.25 μg/mL fungizone (Gibco), 100 U/mL penicillin, and 100m/mL streptomycin. On day 35, cell aggregates were transferred to spinner-flask bioreactors (Corning) and maintained at 56 rpm, in CDM III, consisting of CDM II supplemented with 10% fetal bovine serum (FBS) (GE-Healthcare), 5 μg/mL heparin (Sigma), and 1% Matrigel (Corning). From day 70, organoids were cultured in CDM IV, consisting of CDM III supplemented with B27 supplement (Gibco) and 2% Matrigel. A step-by-step protocol describing the long-term culture of dorsally patterned forebrain organoids is available at Protocol Exchange³³. Over the time course of the differentiations (experimental batches) included in this paper, an estimated 13 batches of FBS, 4 batches of KSR, and 23 batches of Matrigel were used.

We observed that when starting with healthy viable hiPSCs (mycoplasma-free, karyotypically normal and below passage 50) with typical morphological features of undifferentiated cells (tightly packed colonies of round cells with large nuclei and nucleoli) at 80-90% confluency, the efficiency of forebrain cell type generation was ˜90% (93/103 organoids, single-cell RNA-seq and IHC combined efficiency, Extended Data Table 1).

Self-patterned whole-brain organoids were generated as previously described^{3,34}. Dorsal and ventral spheroids were generated using a modification of a previously described protocol¹⁵. Specifically, spheres of pluripotent stem cells were grown for four additional days before neural induction; for ventral spheroids, specification of ventral telencephalic identity was obtained by treatment with the SHH agonist SAG (Selleckhem; 100 nM) from days 7 to 20.

Immunohistochemistry and In Situ Hybridization. Samples were fixed in 4% paraformaldehyde (Electron Microscopy Services), cryoprotected in 30% sucrose solution, embedded in Optimum Cutting Temperature (OCT) compound (Tissue Tek) and cryosectioned (14 μm thickness). Sections were washed with 0.1% TWEEN-20 (Sigma) in Phosphate buffered saline (PBS) (Gibco), blocked for 1 hr at RT with 6% donkey serum (DS) (Sigma)+0.3% Triton X-100 (Sigma) in PBS, and incubated with primary antibodies overnight at 4° C. (diluted with 2.5% DS+0.1% Triton X-100 in PBS). Primary antibodies and dilutions used are specified in Extended Data Table 2. PSD95 and VGluT1 immunohistochemistry (IHC) was performed as in Arlotta et al.²⁰. RNA in situ hybridization was performed using the RNAscope Fluorescent Multiplex Reagent Kit (Advanced Cell Diagnostics) according to the manufacturer's instructions. The probes used are: Hs-EOMES-C2 (429691-C2), Hs-TBR1-C3 (425571-C3), and Hs-Reln (413051) (Advanced Cell Diagnostics).

Microscopy and Image analysis. Images of organoids in culture were taken on an EVOS FL microscope (Invitrogen). Immunofluorescence images were obtained using an LSM 700 inverted confocal microscope (Zeiss) and analyzed with the ZEN Blue/Black 2012 image-processing software.

Dissociation of brain organoids and single-cell RNA-seq. Individual brain organoids were dissociated into a single-cell suspension using the Worthington Papain Dissociation System kit (Worthington Biochemical). A detailed description of the dissociation protocol is available at Protocol Exchange³³. Dissociated cells were resuspended in ice-cold PBS containing 0.04% BSA (Sigma) at a concentration of 1000 cells/μL, and approximately 17,400 cells per channel (to give estimated recovery of 10,000 cells per channel) were loaded onto a Chromium™ Single Cell 3′ Chip (10× Genomics, PN-120236) and processed through the Chromium Controller to generate single-cell GEMs (Gel Beads in Emulsion). Single-cell RNA-Seq libraries were prepared with the Chromium™ Single Cell 3′ Library & Gel Bead Kit v2 (10× Genomics, PN-120237). Libraries from different samples were pooled based on molar concentrations and sequenced on a NextSeq 500 instrument (I lumina) with 26 bases for read 1, 57 bases for read 2 and 8 bases for Index 1. After the first round of sequencing, libraries were re-pooled based on the actual number of cells in each and re-sequenced to give equal number of reads per cell in each sample and to reach a sequencing saturation of at least 50% (in most cases >70%).

Single-cell RNA-seq data analysis. The Cell Ranger 2.0.1 pipeline (10× Genomics)³⁵ was used to align reads from RNA-seq to the GRCh38 human reference genome and produce the associated cell by gene count matrix (Extended Data Table 3). Default parameters were used, except for the ‘--cells’ argument. Unique Molecular Identifier (UMI) counts were analyzed using the Seurat R package v2.3.4³⁶. Cells expressing a minimum of 500 genes were kept, and UMI counts were normalized for each cell by the total expression, multiplied by 1 million, and log-transformed. Seurat's default method for identifying variable genes was used with x.low.cutoff=1, and the ScaleData function was used to regress out variation due to differences in total UMIs per cell. Principal component analysis (PCA) was performed on the scaled data for the variable genes, and significant principal components (PC) were chosen. Cells were clustered in PCA space with a method adapted from²⁷ by finding the 50 nearest neighbors to each cell using R's RANN package³⁷, building a graph with edges between neighbor cells weighted by the Jaccard distance, and performing Louvain clustering on the resulting graph. Variation in the cells was visualized by t-SNE on the significant PCs.

For each dataset of three organoids, differentially expressed genes upregulated in each cluster compared to the rest of the cells were identified using Seurat's implementation of the Model-based Analysis of Single Cell Transcriptomics (MAST) algorithm³⁸ (Extended data Table 1). To identify cell types, genes with a Bonferroni-adjusted p value less than 0.01 and a log Fold Change of at least 0.25 were taken into consideration. Ambient RNA was found to affect clustering distribution in at least two individual batches (Supplementary Note 1), but no correction was needed after comparing data across batches. To do so, we used Seurat's canonical correlation analysis (multi-CCA) procedure for batch correction with default parameters³⁶. Briefly, this identifies vectors along which the datasets correlate, and then aligns values along these vectors to reduce batch variation. T-SNE plots were used to visualize variation in the data after alignment in the top 20 canonical correlation vectors.

Pseudotime analysis was performed using the Monocle package v2.99.1²⁶ with default parameters. UMI counts from 35,000 randomly sampled cells per timepoint were imported into Monocle's CellDataSet object with an expected negative binomial distribution. T-SNE plots were used for visualization, and the “SimplePPT” method was employed to learn a tree-like trajectory between the cells. When ordering the cells along this trajectory, cells previously assigned to the “Cycling Progenitors” cell type were specified as the starting state. Finally, Scrublet v0.1³⁹ was used to assess the effect of droplets that may have contained more than one cell, with an expected doublet rate of 0.1 and a score threshold of 0.39, chosen based on histograms of simulated doublet scores (FIG. 7F).

Comparison to published single-cell RNA-seq data. To compare the cell types found in the dorsally patterned organoids to previously published data from whole-brain organoids³ and endogenous tissue^{18,28-30}, we downloaded UMI count data available from those publications, created Seurat objects, and submitted it to the same normalization, clustering, and visualization pipeline as above. In the case of Fan et al.,²⁹ log(TPM+1) were used instead of UMI counts. To compare variability in cell type proportion between dorsally patterned organoids and whole-brain organoids, intraclass correlation was computed by creating a table of cell type proportions across each individual organoid, and using the icc command in the irr R package v0.84, with model=“twoway”, type=“agreement”, and unit=“single”. To compare cell type classifications between organoid data and fetal human data (as in FIG. 4A and FIG. 9A), cell types as assigned to each cell by Nowakowski et al.¹⁸ were aggregated into more general cell types (after removing clusters not found in organoids: oligodendrocyte precursors, endothelial, and microglia, or with too few cells: unknown and astrocytes) and used to train a Random Forest classifier to distinguish between cell types²⁷. This was done with the tuneRF function in the randomForest R package⁴⁰ with doBest=T, after the data was downsampled to 314 cells per cell type (the size of the smallest cell type). The classifier was then applied to the organoid data to assign organoid cells to one of the human cortex cell types. To calculate correlations between cell types (as in FIG. 3E), we assigned cell types to cells in Nowakowski et al.¹⁸ using the same method that was used for organoids. The Spearman correlation was calculated between the gene expression of highly variable genes in the Nowakowski et al.¹⁸ dataset in the “average cell” in each cell type (calculated with Seurat's AverageExpression function). To measure reproducibility of clusters per individual organoid, human cortex, or mouse cortex sample, data was downsampled to have 659 cells per individual (to match the sample with the fewest cells, adult human cortex 5) before clustering all individuals in a dataset together. Mutual information was calculated between cluster assignments and individuals with the mpmi R package. Z-scores were calculated by creating background distributions for each dataset, by permuting cluster assignments and re-calculating the mutual information score 1,000 times.

Statistics and Reproducibility. A total of 21 single organoids collected at 3 and 6 months, derived from 4 different cell lines, from 6 independent batches (PGP1: 3 batches; HUES66, GM08330, and 11a: 1 batch each; n=3 organoids per batch and timepoint) were profiled by single-cell RNA-seq. Analysis of over 166,242 single cells revealed reproducibility in the diversity of cell types generated “organoid-to-organoid” for 20/21 organoids (˜95% efficiency) (Extended Data Table 1). A total of 82 single organoids, derived from 5 different lines, from 8 independent batches (PGP1: 3 batches; HUES66: 2 batches; GM08330, 11a, and Mito 210: 1 batch each) were analyzed by IHC for expression of EMX1, PAX6, MAP2 at 1 month (n=26/26 positive); 3 months (n=37/37 positive for PAX6 and MAP2; and 36/37 positive for EMX1); and 6 months (n=19/19 positive for PAX6 and MAP2; 11/19 positive for EMX1) (FIG. 1C, FIG. 6B, and Extended Data Table 1). IHC for CTIP2 and SATB2 was performed on 3 month organoids derived from 5 different cell lines, from 8 independent batches (PGP1: 3 batches; HUES66: 2 batches; GM08330, 11a, and Mito 210: 1 batch each) (n=32/37 positive) (FIG. 1C, FIG. 6B). IHC for GFAP and S100B (FIG. 2C) was performed on 6 month organoids derived from 1 cell line (PGP1, 1 batch) (n=3/3 positive). IHC for VGluT1 and PSD95 (FIG. 3c) was performed on 3 month organoids from 2 cell lines, from 2 independent batches (PGP1 and 11a: 1 batch each) (n=8/8 positive). IHC for SOX2 (FIG. 6B) was performed on 3 month organoids from 5 cell lines, from 7 batches (PGP1: 2 batches; HUES66: 2 batches; 11a, GM08330, and Mito210: 1 batch) (n=34/34 positive). IHC for Ki67 (FIG. 6B) was performed on 3 month organoids from 4 cell lines, from 4 batches (PGP1, 11a, HUES66, and GM08330: 1 batch each) (n=22/22 positive). IHC for FOXG1, HOPX, TBR1, and TBR2 at 3 months and 6 months (FIG. 6B) was performed on organoids from one cell line (PGP1: 1 batch) (n=7/7 positive). IHC for HOPX at 3 months was also performed on two additional cell lines (HUES66 and GM08330: 1 batch each) (n=12/12 positive). IHC for cleaved Caspase 3 (FIG. 7E) was performed on organoids from one batch of the PGP1 cell line, at 3, 4, 5 and 6 months (n=3 for each timepoint). Overall, expression of MAP2, EMX1, PAX6, CTIP2, and SATB2 was assessed at 1, 3 and 6 months on organoids from each independent batch, and at 2, 4 and 5 months for one batch of PGP1 (FIG. 5B).

RNA in situ Hybridization for EOMES (TBR2), Reelin, and TBR1 was performed on 3 month organoids from 4 cell lines (PGP1, 11a, GM08330, HUES66), from 4 batches (n=22/22 positive). No statistical methods were used to predetermine the sample size.

Data availability. Single-cell RNA-seq data that support the findings of this study have been deposited at Gene Expression Omnibus, accession number GSE129519, and at the Single Cell Portal (portals.broadinstitute.org/single_cell/study/reproducible-brain-organoids). The reference datasets used for comparison are available in the Gene Expression Omnibus at accession numbers GSE86153, GSE116470, and GSE103723, or in dbGaP at accession phs000989.v3, and phs000424.v8.pl.

Code availability. The code used for data analysis is available on GitHub (https://github.com/AmandaKedaigle/BrainOrganoidsReproducibility).

Extended Data Table 2
Primary antibodies used for immunofluorescence.

| Primary | | | Catalog | |
|---|---|---|---|---|
| Antibody | Host | Manufacturer | Number | Dilution |
| Cleaved | Rabbit | Cell Signaling | 9661 | 1/300 |
| Caspase 3 | | Technologies | | |
| CTIP2 | Rat | Abcam | AB18465 | 1/100 |
| EMX1 | Rabbit | Atlas Antibodies | HPA006421 | 1/50 |
| FOXG1 | Rabbit | Abcam | 196868 | 1/100 |
| GFAP | Mouse | Sigma-Aldrich | G3893 | 1/400 |
| HOPX | Rabbit | Sigma-Aldrich | HPA030180 | 1/2,500 |
| Ki67 | Mouse | BD Biosciences | 550609 | 1/400 |
| MAP2 | Chicken | Abcam | AB5392 | 1/5,000 |
| PAX6 | Rabbit | Biolegend | 901301 | 1/400 |
| PSD95 | Rabbit | Thermo Fisher | 51-6900 | 1/350 |
| S100B | Rabbit | Abcam | AB41548 | 1/2,000 |
| SATB2 | Mouse | Abcam | AB51502 | 1/50 |
| SOX2 | Goat | RD Systems | AF2018 | 1/50 |
| TBR1 | Rabbit | Abcam | AB31940 | 1/500 |
| TBR2 | Rabbit | Millipore | AB2283 | 1/2,000 |
| VGluT1 | Guinea Pig | Millipore | AB5905 | 1/2,000 |

EXTENDED DATA TABLE 3
Statistics from single-cell RNA-sequencing. Columns 3-6 represent
initial statistics reported from the CellRanger program, for
consistency with prior studies; columns 7-9 represent final
data after full quality control, as used in the analyses.

| | | | | Mean | Reads Mapped |
|---|---|---|---|---|---|
| | | | | Reads/ | Confidently to |
| | | | Total Reads | Cell | Transcriptome |
| 3 Mo. | PGP1 | Org #1 | 282,380,462 | 47,610 | 45.80% |
| | (batch 1) | Org #2 | 308,237,385 | 44,847 | 47.00% |
| | | Org #3 | 232,194,411 | 46,429 | 45.30% |
| | PGP1 | Org #4 | 320,293,451 | 35,627 | 48.80% |
| | (batch 2) | Org #5 | 290,649,261 | 38,048 | 49.60% |
| | | Org #6 | 312,301,428 | 28,326 | 49.00% |
| | HUES66 | Org #7 | 357,410,387 | 33,993 | 53.90% |
| | | Org #8 | 360,164,748 | 35,362 | 52.70% |
| | | Org #9 | 458,590,387 | 37,329 | 53.30% |
| 6 Mo. | 11a | Org #10 | 328,096,843 | 35,504 | 50.80% |
| | | Org #11 | 309,099,766 | 36,343 | 52.00% |
| | | Org #12 | 399,350,440 | 50,589 | 49.20% |
| | GM08330 | Org #13 | 178,317,524 | 30,898 | 50.00% |
| | | Org #14 | 175,558,635 | 32,271 | 49.60% |
| | | Org #15 | 124,971,025 | 30,743 | 51.00% |
| | PGP1 | Org #16 | 420,342,563 | 49,850 | 52.50% |
| | (batch 1) | Org #17 | 300,940,886 | 47,496 | 52.80% |
| | | Org #18 | 314,512,990 | 48,565 | 56.80% |
| | PGP1 | Org #19 | 249,696,784 | 30,376 | 50.60% |
| | (batch 2) | Org #20 | 150,922,738 | 23,055 | 47.80% |
| | | Org #21 | 347,029,455 | 31,479 | 58.00% |

| | | | | | Mean | Median |
|---|---|---|---|---|---|---|
| | | | | | Mapped | Mapped |
| | | | Median | Cells | Reads in | Reads in |
| | | | Genes/ | Passing | Passing | Passing |
| | | | Cell | QC | Cells | Cells |
| 3 Mo. | PGP1 | Org #1 | 2,086 | 5,924 | 17,915 | 16,290 |
| | (batch 1) | Org #2 | 1,898 | 6,858 | 18,010 | 15,555 |
| | | Org #3 | 2,055 | 4,992 | 17,926 | 15,485 |
| | PGP1 | Org #4 | 2,078 | 8,989 | 14,221 | 12,146 |
| | (batch 2) | Org #5 | 1,988 | 7,639 | 15,012 | 11,545 |
| | | Org #6 | 1,501 | 11,018 | 11,481 | 9,638 |
| | HUES66 | Org #7 | 1,741 | 10,509 | 15,599 | 13,346 |
| | | Org #8 | 1,709 | 10,175 | 15,336 | 13,032 |
| | | Org #9 | 1,589 | 12,275 | 17,141 | 15,263 |
| 6 Mo. | 11a | Org #10 | 1,290 | 9,237 | 14,958 | 10,947 |
| | | Org #11 | 1,330 | 8,495 | 15,786 | 11,773 |
| | | Org #12 | 1,438 | 7,886 | 20,607 | 15,158 |
| | GM08330 | Org #13 | 1,723 | 5,759 | 11,255 | 8,007 |
| | | Org #14 | 1,758 | 5,436 | 11,513 | 8,362 |
| | | Org #15 | 1,724 | 4,061 | 10,635 | 7,485 |
| | PGP1 | Org #16 | 1,477 | 8,425 | 20,150 | 14,760 |
| | (batch 1) | Org #17 | 1,727 | 6,327 | 19,659 | 14,726 |
| | | Org #18 | 1,501 | 6,461 | 22,105 | 16,655 |
| | PGP1 | Org #19 | 1,472 | 8,215 | 12,331 | 9,489 |
| | (batch 2) | Org #20 | 1,581 | 6,539 | 9,253 | 7,102 |
| | | Org #21 | 2,053 | 11,022 | 17,026 | 15,005 |

### REFERENCES

1. Silbereis, J. C., Pochareddy, S., Zhu, Y., Li, M. & Sestan, N. The Cellular and Molecular Landscapes of the Developing Human Central Nervous System. Neuron 89, 248-268 (2016).
2. Quadrato, G., Brown, J. & Arlotta, P. The promises and challenges of human brain organoids as models of neuropsychiatric disease. Nat. Med. 22, 1220-1228 (2016).
3. Quadrato, G. et al. Cell diversity and network dynamics in photosensitive human brain organoids. Nature 545, 48-53 (2017).
4. Lancaster, M. A. et al. Cerebral organoids model human brain development and microcephaly. Nature 501, 373-379 (2013).
5. Kadoshima, T. et al. Self-organization of axial polarity, inside-out layer pattern, and species-specific progenitor dynamics in human ES cell-derived neocortex. Proc. Natl. Acad. Sci. 110, 20284-20289 (2013).
6. Yoon, S. J. et al. Reliability of human cortical organoid generation. Nat. Methods 16, 75-78 (2018).
7. Pollen, A. A. et al. Establishing Cerebral Organoids as Models of Human-Specific Brain Evolution. Cell 176, 743-756.e17 (2019).
8. Camp, J. G. et al. Human cerebral organoids recapitulate gene expression programs of fetal neocortex development. Proc. Natl. Acad. Sci. 112, 15672-15677 (2015).
9. Qian, X. et al. Brain-Region-Specific Organoids Using Mini-bioreactors for Modeling ZIKV Exposure. Cell 165, 1238-1254 (2016).
10. Lancaster, M. A. et al. Guided self-organization and cortical plate formation in human brain organoids. Nat. Biotechnol. 35, 659-666 (2017).
11. Bagley, J. A., Reumann, D., Bian, S., Levi-Strauss, J. & Knoblich, J. A. Fused cerebral organoids model interactions between brain regions. Nat. Methods 14, 743-751 (2017).
12. Xiang, Y. et al. Fusion of regionally-specified hPSC-derived organoids models human brain development and interneuron migration. Cell Stem Cell 21, 383-398.e7 (2017).
13. Birey, F. et al. Assembly of functionally integrated human forebrain spheroids. Nature 545, 54-59 (2017).
14. Bershteyn, M. et al. Human iPSC-Derived Cerebral Organoids Model Cellular Features of Lissencephaly and Reveal Prolonged Mitosis of Outer Radial Glia. Cell Stem Cell 20, 435-449.e1-e4 (2017).
15. Rigamonti, A. et al. Large-scale production of mature neurons from human pluripotent stem cells in a three-dimensional suspension culture system. Stem Cell Reports 6, 993-1008 (2016).
16. Church, G. M. The Personal Genome Project. Mol. Syst. Biol. 1, E1-E3 (2005).
17. Pollen, A. A. et al. Molecular Identity of Human Outer Radial Glia during Cortical Development. Cell 163, 55-67 (2015).
18. Nowakowski, T. J. et al. Spatiotemporal gene expression trajectories reveal developmental hierachies of the human cortex. Science (80-.). 358, 1318-1323 (2017).
19. Molyneaux, B. J. et al. Novel Subtype-Specific Genes Identify Distinct Subpopulations of Callosal Projection Neurons. J. Neurosci. 29, 12343-12354 (2009).
20. Arlotta, P. et al. Neuronal subtype-specific genes that control corticospinal motor neuron development in vivo. Neuron 45, 207-221 (2005).
21. Zhang, Y. et al. Purification and Characterization of Progenitor and Mature Human Astrocytes Reveals Transcriptional and Functional Differences with Mouse. Neuron 89, 37-53 (2016).
22. Darmanis, S. et al. A survey of human brain transcriptome diversity at the single cell level. Proc. Natl. Acad. Sci. 112, 7285-7290 (2015).
23. Lodato, S. & Arlotta, P. Generating Neuronal Diversity in the Mammalian Cerebral Cortex. Annu. Rev. Cell Dev. Biol. 31, 699-720 (2015).
24. Greig, L. C., Woodworth, M. B., Galazo, M. J., Padmanabhan, H. & Macklis, J. D. Molecular logic of neocortical projection neuron specification, development and diversity. Nat. Rev. Neurosci. 14, 755-769 (2013).
25. Kohwi, M. et al. A Subpopulation of Olfactory Bulb GABAergic Interneurons Is Derived from Emx1—and D1×5/6-Expressing Progenitors. J. Neurosci. 27, 6878-6891 (2007).
26. Qiu, X. et al. Reversed graph embedding resolves complex single-cell trajectories. Nat. Methods 14, 979-982 (2017).
27. Shekhar, K. et al. Comprehensive Classification of Retinal Bipolar Neurons by Single-Cell Transcriptomics. Cell 166, 1308-1323.e30 (2016).
28. Habib, N. et al. Massively parallel single-nucleus RNA-seq with DroNc-seq. Nat. Methods 14, 955-958 (2017).
29. Fan, X. et al. Spatial transcriptomic survey of human embryonic cerebral cortex by single-cell RNA-seq analysis. Cell Res. 28, 730-745 (2018).
30. Saunders, A. et al. Molecular Diversity and Specializations among the Cells of the Adult Mouse Brain. Cell 174, 1015-1030.e16 (2018).
31. Sheridan, S. D. et al. Epigenetic characterization of the FMR1 gene and aberrant neurodevelopment in human induced pluripotent stem cell models of fragile X syndrome. PLoS One 6, e26203 (2011).
32. Sugathan, A. et al. CHD8 regulates neurodevelopmental pathways associated with autism spectrum disorder in neural progenitors. Proc. Natl. Acad. Sci. 111, E4468-E4477 (2014).
33. Velasco, S., Paulsen, B. & Arlotta, P. Highly reproducible human brain organoids recapitulate cerebral cortex cellular diversity. Nat. Protoc. Exch. (2019).
34. Quadrato, G., Sherwood, J. L. & Arlotta, P. Long term culture and electrophysiological characterization of human brain organoids. Protoc. Exch. (2017). doi:10.1038/protex.2017.049
35. Zheng, G. X. Y. et al. Massively parallel digital transcriptional profiling of single cells. Nat. Commun. 8, 1-12 (2017).
36. Butler, A., Hoffman, P., Smibert, P., Papalexi, E. & Satija, R. Integrating single-cell transcriptomic data across different conditions, technologies, and species. Nat. Biotechnol. 36, 411-420 (2018).
37. Arya, S., Mount, D., Kemp, S. E. & Jefferis, G. RANN: Fast Nearest Neighbour Search (Wraps ANN Library) Using L2 Metric. R package version 2.6. (2018).
38. McDavid, A., Finak, G. & Yajima, M. MAST: Model-based Analysis of Single Cell Transcriptomics. R package version 1.6.1. (2018).
39. Wolock, S. L., Lopez, R. & Klein, A. M. Scrublet: computational identification of cell doublets in single-cell transcriptomic data. bioRxiv 1-18 (2018).
40. Liaw, A. & Wiener, M. Classification and Regression by randomForest. R News 2, 18-22 (2002).

Detailed Protocol

Reagents and Equipment:

Reagents

2-mercaptoethanol (ThermoFisher Scientific, #21985023)

B-27 Supplement, 50× (ThermoFisher Scientific, #17504044)

Chemically Defined Lipid Concentrate (ThermoFisher Scientific, #11905031)

DMEM/F-12 (ThermoFisher Scientific, #11330032)

DMSO (Sigma, #D2650)

Fungizone/Amphotericin B (ThermoFisher Scientific, #15290018)

Geltrex, LDEV-Free, hESC-Qualified, Reduced Growth Factor Basement Membrane

Matrix (ThermoFisher Scientific, #A1413301)

Gentle Cell Dissociation Reagent (Stemcell Technologies, #07174)

Glasgow MEM, G-MEM (ThermoFisher Scientific, #11710035)

GlutaMAX Supplement (ThermoFisher Scientific, #35050061)

UltraPure DNase/RNase-free Distilled Water (ThermoFisher Scientific, #10977015)

Heparin (Sigma, #H3149)

Hyclone Fetal Bovine Serum (ThermoFisher Scientific, #SH30070.03)

IWR1 (Calbiochem/Millipore, #681669)

KnockOut Serum Replacement (ThermoFisher Scientific, #10828028)

Matrigel (#356234, Corning)

MEM Non-Essential Amino Acids Solution (ThermoFisher Scientific, #11140050)

mTeSR1 (Stemcell Technologies, #85850)

MycoAlert PLUS mycoplasma detection kit (Lonza, #LT07705)

N-2 Supplement, 100× (Life Technologies, #17502048)

Papain Dissociation System kit (Worthington, #LK003153)

PBS, phosphate buffered saline (ThermoFisher Scientific, #10010023)

Penicillin/Streptomycin Solution 100× (Corning, #30002C1)

ROCK inhibitor, Y-27632 (EMD Millipore, #SCM075)

SB 431542 (Stemcell Technologies, #72234)

Sodium Pyruvate (ThermoFisher Scientific, #11360070)

StemPro Accutase Cell Dissociation Reagent (ThermoFisher Scientific, #A1110501)

Trypan blue solution, 0.4% (ThermoFisher Scientific, #15250061)

Equipment

2 mL Aspirating Pipette (VWR, #357558)

5 mL Serological Pipette (VWR, #89130896)

10 mL Serological Pipette (VWR, #89130898)

15 mL Conical Tubes, Polystyrene (VWR, #352097)

50 mL Conical Tubes, Polystyrene (VWR, #352070)

50 mL Serological Pipette (VWR, #89130902)

60 mm TC-Treated Cell Culture Dish (Corning, #353002)

10 mm Ultra Low Culture Dish (Corning, #3262)

Cell lifter (Corning, #3008)

125 mL spinner flasks (Corning, #3152)

Centrifuges

CO₂ incubator

Corning Falcon Test Tube with Cell Strainer Snap Cap (Corning, #352235)

Countess II Automated Cell Counter (ThermoFisher Scientific, #AMQAX1000)

Steriflip-GP Sterile Centrifuge Tube Top Filter Unit (Millipore, #SCGP00525)

Filter Bottles 0.22 μm 150 mL (Corning, #CLS431155)

Filter Bottles 0.22 μm 250 mL (Corning, #CLS430768)

Hemocytometer

Magnetic stirrer (ChemGlass, #CLS-4100-09)

Microscope, inverted

Pipettes

Prime surface 96V plate, v-bottom (Sbio Japan, #MS9096VZ)

Razor blades (VWR, #55411050)

Reagent reservoir (VWR, #89094680)

Digital CO₂-resistant orbital shaker (ThermoFisher Scientific, #88881101)

Procedure:

Reagents setup

Reagents stock

Y-27632 (ROCK Inhibitor)

Reconstitute 5 mg in 2.96 mL of sterile water to make a 5 mM stock solution. Store at −20° C. for up to 1 year. Avoid multiple freeze-thaw cycles.

SB431542 (Activin/BMP/TGF-β pathway inhibitor)

Reconstitute 10 mg in 2.6 mL of DMSO to make a 10 mM stock solution. Store at −20° C. for up to 1 year. Avoid multiple freeze-thaw cycles.

IWR1 (Wnt Inhibitor)

Reconstitute 10 mg in 814 μl of DMSO to make a 30 mM stock solution. Store at −20° C. for up to 1 year. Avoid multiple freeze-thaw cycles.

Heparin

The concentration used here is measured in mg/ml, however the manufacturer reports quantity in units. The potency (units/mg) is lot-specific; the value can be found on the certificate of analysis (it is typically over 140 units per mg). The number of mg required is calculated by dividing the number of units of the batch by the potency in units per mg. Reconstitute the batch in the volume of water needed to make a 10 mg/mL stock solution. Reconstituted solutions can be stored at 2-8° C. for up to 2 years, if sterile filtered through a 0.22 μm filter.

Media Preparation

Cortical Differentiation Medium I—CDM I [Day 0-18] (Quantity Per 100 mL Medium)

Glasgow MEM, G-MEM (77 mL)

KnockOut Serum Replacement (20 mL—final 20%) MEM Non-Essential Amino Acids Solution (1 mL) Sodium pyruvate (1 mL)

2-mercaptoethanol (182 μL of 55 mM stock—final 0.1 mM)

Penicillin/Streptomycin Solution (1 mL)

After mixing all reagents, filter medium using a 0.22 μm filter bottle

Cortical Differentiation Medium II—CDM II [Day 18-35] (quantity per 100 mL medium)

DMEM/F-12 (96 mL)

GlutaMAX Supplement (1 mL)

N-2 Supplement (1 mL)

Chemically Defined Lipid Concentrate (1 mL)

Fungizone (100 μL of 250m/ml stock—final 0.25 μg/mL) Penicillin/Streptomycin Solution (1 mL)

After mixing all reagents, filter medium using a 0.22 μm filter bottle

Cortical Differentiation Medium III—CDM III [Day 35-70] (quantity per ˜200 mL medium)

DMEM/F-12 (170 mL)

Hyclone Fetal Bovine Serum (20 mL—final 10%) Chemically Defined Lipid Concentrate (2 mL) GlutaMAX Supplement (2 mL)

N-2 Supplement (2 mL)

Heparin (100 μL of 10 mg/mL—final 5 μg/mL)

Fungizone (200 μL of 250m/mL stock—final 0.25 μg/mL) Penicillin/Streptomycin Solution (2 mL)

After mixing all reagents, filter medium using a 0.22 μm filter bottle.

Add Matrigel to the medium, after filtration, to obtain a final concentration of 1% (2 mL for 200 mL). If Matrigel is added when the medium is warm, it will turn into a gel and not properly mix with the solution.

Cortical Differentiation Medium IV—CDM IV [Day 70 On] (Quantity Per ˜200 mL Medium)

DMEM/F-12 (166 mL)

Hyclone Fetal Bovine Serum (20 mL—final 10%) Chemically Defined Lipid Concentrate (2 mL) GlutaMAX Supplement (2 mL)

N-2 Supplement (2 mL)

B-27 Supplement (4 mL)

Heparin (100 uL of 10 mg/mL stock—5 μg/mL) Fungizone (200 μL of 250 μg/L stock—final 0.25 μg/mL) Penicillin/Streptomycin Solution (2 mL)

After mixing all reagents, filter medium using a 0.22 μm filter bottle

Add Matrigel to the medium, after filtration, to obtain a final concentration of 2% (4 mL for 200 mL). If Matrigel is added when the medium is warm, it will turn into a gel and not properly mix with the solution.

Generation of Reproducible Human Brain Organoids

Maintenance of human pluripotent stem cells (hPSCs)

Maintain feeder-free hPSCs on TC-treated cell culture dishes pre-coated with Geltrex, in mTESR1 medium with 100 U/mL penicillin and 100m/mL streptomycin, in a humidified incubator at 37° C. and 5% CO₂.

Detailed protocol for thawing, maintenance, and passaging of hPSCs can be found in Arlotta et al., 2017^{[5]}.

CRITICAL STEP: It is recommended to routinely confirm karyotype stability (suggested: every 5 passages) and make sure that stem cells are mycoplasma-free (on a weekly basis). MycoAlert PLUS Mycoplasma Detection Kit can be used to detect mycoplasma contamination. Single-cell dissociation should be avoided during passaging. The use of non-enzymatic methods to detach cell clumps, for instance Gentle Cell Dissociation Reagent, is recommended.

Day 0—Cell Aggregate Formation in CDM I Media

Start with a 60 mm Cell Culture Dish of 80-90% confluent hPSCs.

CRITICAL STEP: Optimal cell density is crucial for the positive outcome of the protocol. For the generation of brain organoids, it is recommended to use hPSCs between passage 20 and 45, mycoplasma-free, and karyotypically normal. Use only healthy viable hPSCs, with typical morphological features of pluripotent cells (tightly packed colonies of round cells with large nuclei and nucleoli), that have no sign of differentiation. Optimal stem cell culture practice and attention to details are essential requirements for the formation of healthy dorsal forebrain organoids.

Before starting cell dissociation, prepare the following mixes:

2a. Washing Medium I [volume for one 60 mm TC-Treated Cell Culture Dish]

mTeSR1 (10 mL)

ROCK inhibitor (20 μL of 5 mM stock—final 10 μM)

2b. Washing Medium II [volume for one 60 mm TC-Treated Cell Culture Dish]

CDM I (11 mL)

ROCK inhibitor (22 μL of 5 mM stock—final 10 μM)

2c. Seeding Medium [volume for one Prime surface 96V plate]

CDM I (10 mL)

ROCK inhibitor (40 μL of 5 mM stock—final 20 μM)

IWR1 (1 μL of 30 mM stock—3 μM)

SB 431542 (5 μL of 10 mM stock —final 5 μM—final 5 μM)

Gently aspirate the media and wash the cells with 5 mL of PBS.

Immediately after removing the PBS, add 4 mL of Accutase and incubate for approximately 4-5 minutes at 37° C.

CRITICAL STEP: ROCK inhibitor is added to Washing Medium I and II to increase single cell survival during the dissociation process^{[6]}. Check the cells under the microscope after 4 minutes to ensure they are completely detached. Different incubation times may be required depending on cell confluence or for different hPSCs.

Use a 1 mL pipette to gently dissociate the cells in accutase.

CRITICAL STEP: Be gentle and avoid rough pipetting to prevent damage to cells, which can lead to cell death.

Add 5 mL of Washing Medium I directly to the dish, to dilute the Accutase suspension containing the dissociated cells and transfer content to a 15 mL conical tube. Immediately add other 5 mL of Washing Medium I to the conical tube and gently homogenize the solution using a 5 mL pipette.

Centrifuge the dissociated cells at 140 g for 4 minutes.

Gently aspirate the supernatant and resuspend the cells in 10 mL of Washing Medium II.

Centrifuge the cells at 140 g for 4 minutes.

Aspirate the supernatant and resuspend the cells in 1 mL of Washing Medium II. Use a 1 mL pipette to gently dissociate the cells into a single-cell suspension. Immediately after resuspending the cells, dilute and count them, as follows:

11a. Dilution I (1:10)—Add 10 μL of cell suspension into 90 μL of PBS and mix well (if the initial number of cells is low, a 1:5 dilution, e.g., 10 μL into 40 ul of PBS may be preferable);

11b. Dilution II (1:2)—Add 10 μL of Dilution I into 10 μL of Trypan blue solution and mix well;

11c. Use an automatic cell counter or a hemocytometer to count the cells. If using the latter, remember to consider both dilutions for the calculation of cell concentration.

Dilute the proper volume of cell suspension (containing 900,000 cells) obtained above into 10 mL of Seeding Medium and gently mix the solution. Plate 9,000 cells/well in a Prime surface 96V plate (100 μL/well). Keep the plate in a humidified tissue culture incubator at 37° C. and 5% CO₂.

Day 0—Observe the 96 multiwell plate under the microscope to confirm aggregate formation.

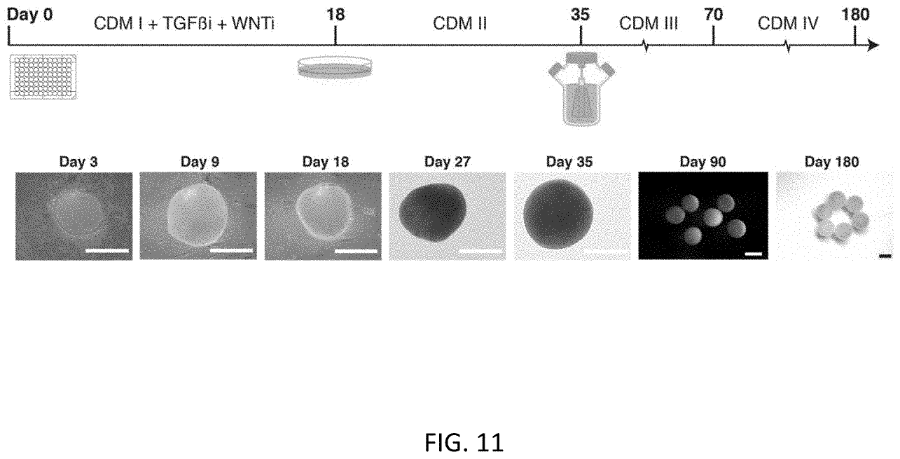

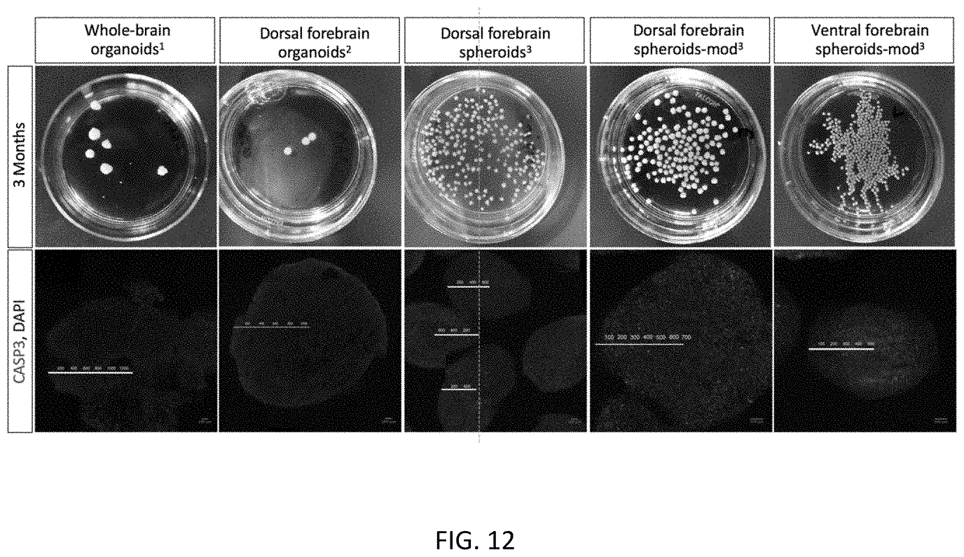

CRITICAL STEP: Aggregates with defined and homogeneous borders should be visible, although a few dead cells might appear around the EB (FIG. 11). The presence of multiple small aggregates per well suggests that the dissociation process was not performed properly, or that the number of cells seeded was not correct. If this occurs, it is not recommended to proceed any further with this batch.

Day 3—Media change with CDM I Media

Observe the aggregates under the microscope.

Prepare the following mix:

CDM I (10 mL)

ROCK inhibitor (40 μL of 5 mM stock—final 20 μM)

IWR1 (1 μL of 30 mM stock—final 3 μM)

SB 431542 (5 μL of 10 mM stock—final 5 μM)

Using a multichannel pipette, add 100 μL of mix to each well.

CRITICAL STEP: before adding fresh medium, gently agitate the medium inside each well by using a multi-channel pipette set to 50 μl: withdraw media and then return it to the well. Do this only once (to avoid aspirating the floating aggregates with the pipette). Make sure not to touch the aggregates at the bottom of the well. This step helps detach dead cells that are surrounding the aggregate as a result of the dissociation step.

Return the plate to a humidified tissue culture incubator at 37° C. and 5% CO₂.

Day 6—Media change with CDM I Media

Prepare the following mix:

CDM I (10 mL)

IWR1 (1 μL of 30 of mM stock—final 3 μM)

SB 431542 (5 μL of 10 mM stock—final 5 μM)

Remove 80 μL of medium from each well of the multiwell plate. Using a multichannel pipette, add 100 μL of the mix to each well. Return the plate to a humidified tissue culture incubator at 37° C. and 5% CO₂.

CRITICAL STEP: Before removing medium, agitate the medium inside each well by using a multi-channel pipette set to 80 μl: withdraw media and then return it to the well. Do this only once (to avoid aspirating the aggregates with the pipette). Make sure not to touch the aggregates at the bottom of the well.

Day 9—Media change with CDM I Media

Repeat procedure as in Day 6.

Day 12—Media change with CDM I Media

Repeat procedure as in Day 6.

Day 15—Media change with CDM I Media

Repeat procedure as in Day 6.

Day 18—Transfer aggregates to 100 mm ultra-low attachment dish filled with CDM H Media

Add 12 mL of CDM II to each of 2 low ultra-low attachment 100 mm plates. Using a 200 μL pipette set to 60-65 μL, transfer approximately 48 aggregates (half of the multiwell plate) to the 100 mm plate (2 plates in total for one multiwell; final volume per plate=15 mL).

CRITICAL STEP: Make sure to use wide-bore 200 μL tips, or cut a standard 200 μL tip with sterile scissors to obtain an opening of 1-1.5 mm in diameter. Using standard tips or other tools to transfer the aggregates may damage them and cause cell death.

Maintain the plate on a CO₂-resistant orbital shaker at 70 rpm inside a humidified tissue culture incubator at 37° C. and 5% CO₂.

Day 21—Media change with CDM II Media

Carefully aspirate most of the medium in each plate and replace it with 15 mL of fresh CDM II.

CRITICAL STEP: To change the medium, tilt the dish and allow the aggregates to settle towards the edge. Then, carefully aspirate as much medium as possible without disturbing the aggregates. A small amount of medium can be left to prevent the aggregates from drying out.

Return the plate to the orbital shaker at 70 rpm inside a humidified tissue culture incubator at 37° C. and 5% CO₂.

Day 24—Media change with CDM II Media

Repeat procedure as in Day 21.

Day 27—Media change with CDM II Media

Repeat procedure as in Day 21.

Day 30—Media change with CDM II Media

Repeat procedure as in Day 21.

Day 33—Media change with CDM II Media

Repeat procedure as in Day 21.

Day 35—Transfer to spinner flask in CDM III Media

Carefully unpack a spinner flask in a sterile environment and fill it with 100 mL of fresh CDM III Media. Use a 50 mL pipette to gently transfer the aggregates to the spinner flask. Transfer up to 96 aggregates per flask. Maintain cultures on a magnetic stirrer at 56 rpm (note change in speed) inside a humidified tissue culture incubator at 37° C. and 5% CO₂.

CRITICAL STEP: Verify that organoids contain dorsal forebrain cell types via immunohistochemistry (IHC) (see Troubleshooting).

Days 42—Media change with CDM III

Replace the medium by completely aspirating it and adding fresh CDM III.

CRITICAL STEP: To change medium, remove the cap from one of the arms of the spinner flask to insert the 1 mL aspirating pipette; avoid removing the main top cap of the spinner flask, to reduce the chance of contamination.

Return spinner flasks to a magnetic stirrer at 56 rpm inside a humidified tissue culture incubator at 37° C. and 5% CO₂.

Days 49—Media change with CDM III

Repeat procedure as in Day 42.

Days 56—Media change with CDM III

Repeat procedure as in Day 42.

Days 63—Media change with CDM III

Repeat procedure as in Day 42.

Day 70 and on—Media change with CDM IV

From Day 70 onwards, use the same procedure for changing media, but substitute CDM III with CDM IV. Maintain spinner flasks on a magnetic stirrer at 56 rpm inside a humidified tissue culture incubator at 37° C. and 5% CO₂.

Single-cell dissociation of brain organoids for single-cell RNA Sequencing (10× Genomics Chromium platform)

The following protocol is a modification of the Worthington Papain Dissociation kit manufacturer's protocol, as used in previous publications^{[3,8]}.

Before starting the protocol, reconstitute the following reagents:

1a. Add 5 mL of Earle's medium into Papain vial (5 ea/kit—use 1 vial for 2 organoids);

1b. Add 500 μL of Earle's medium into DNase vial (5 ea/kit—use 1 vial for 2 organoids);

1c. Add 32 mL of Earle's medium into Inhibitor vial (1 ea/kit—use 1 vial for up to 10 organoids).

Mix 500 μL of reconstituted DNase with 5 mL of reconstituted Papain.

CRITICAL STEP: Mix gently as DNase is sensitive to shear denaturation.

Gently transfer each organoid to an individual 60 mm dish using a 50 mL pipette. Carefully aspirate excess media and immediately add 2.5 mL of Papain+DNase solution per 60 mm plate with a 5 mL pipette. Using a new, sterile razor blade, mince organoids into small pieces (<1 mm). Transfer the plate to an orbital shaker at 70 rpm inside a humidified tissue culture incubator at 37° C. and 5% CO₂ for 30 minutes. Use a 1 mL pipette to gently dissociate and break up minced pieces.

CRITICAL STEP: Rough pipetting can damage cells and affect cell-type representation in the single-cell RNA sequencing analysis.

Return the plate to the orbital shaker at 70 rpm inside a humidified tissue culture incubator at 37° C. and 5% CO₂ for 10 more minutes.

CRITICAL STEP: The incubation time in papain solution required for dissociation may vary with organoid age and between organoids generated from different cell lines. If most of the cells are already in suspension, skip the step of returning the plate to the orbital shaker at 70 rpm inside a humidified tissue culture incubator at 37° C. and 5% CO₂ for 10 more minutes.

During this time, add 5 mL of Earle's medium plus 3 mL of reconstituted Inhibitor solution to a 15 mL conical tube (prepare 1 tube per organoid). Using a 10 mL pipette, gently pipette the minced pieces up-and-down 10 times. Transfer the entire volume to an empty 15 mL conical tube and wait for the debris to settle (1-3 minutes). Using a 10 mL pipette, transfer the cell suspension (avoiding debris) to the 15 ml conical tube prepared above. Tightly close the tube, and carefully invert it a few times to mix. Centrifuge the cells at 300 g for 7 minutes. Carefully aspirate the supernatant, and use a 1 mL pipette to gently resuspend the cells in 500-1,000 μL of 1×PBS. Add 500 μL of fresh 1×PBS to the mesh cap of a 35 μm nylon mesh filter tube to wet the mesh. Remove the buffer completely and then apply the resuspended cells, allowing them to pass through the filter (use 1 tube per organoid). Dilute and count the filtered cells, as follows:

11a. Dilution I (1:10)—Add 10 μL of cell suspension into 90 μL of 1×PBS and mix well; 11b. Dilution II (1:2)—Add 10 μL of Dilution I into 10 μL of Trypan Blue and mix well;

11c. Use an automatic cell counter or a hemocytometer to count the cells. If using the latter, remember to consider both dilutions in the cell count.

Resuspend the cells at 1,000 cells/μL. Add BSA to a final concentration of 0.04%.

Anticipated Results:

In general, when healthy (karyotipically normal, mycoplasma-free) viable hPSCs, 80-90% confluent, showing good pluripotent morphology (no signs of differentiation) are used, the efficiency of forebrain cell type generation by using this protocol is ˜90-95%^{[7]}.

### REFERENCES

1. Paşca, S. P. et al. The rise of three-dimensional human brain cultures. Nature 553 (7689), 437-445 (2018).
2. Brown, J. et al. Studying the Brain in a Dish: 3D Cell Culture Models of Human Brain Development and Disease. Curr Top Dev Biol. 129, 99-122 (2018).
3. Quadrato, G. et al. Cell diversity and network dynamics in photosensitive human brain organoids. Nature 545, 48-53 (2017).
4. Kadoshima, T. et al. Self-organization of axial polarity, inside-out layer pattern, and species-specific progenitor dynamics in human ES cell-derived neocortex. PNAS 110, 20284-20289 (2013).
5. Arlotta, P. et al. Long-term culture and electrophysiological characterization of human brain organoids, Protocol Exchange https://dx.doi.org/10.1038/protex.2017.049 (2017).
6. Watanabe, K. et al. A ROCK inhibitor permits survival of dissociated human embryonic stem cells. Nat. Biotechnol. 25(6), 681-682 (2007).
7. Velasco, S. et al. Individual brain organoids reproducibly form cell diversity of the human cerebral cortex. Nature. In press. (2019).
8. Arlotta, P. et al. Neuronal subtype-specific genes that control corticospinal motor neuron development in vivo. Neuron. 45(2), 207-221 (2005).

1. A dorsal forebrain organoid having a core, wherein the core comprises less than 25% apoptotic or hypoxic cells.

2. The dorsal forebrain organoid of claim 1, wherein the core comprises less than 20%, 15%, 10%, 5%, 1%, or 0.1% apoptotic or hypoxic cells.

3.-7. (canceled)

8. The dorsal forebrain organoid of claim 1, wherein the organoid has been cultured for about 3 months.

9. The dorsal forebrain organoid of claim 8, wherein the organoid comprises one or more of corticofugal projection neurons, callosal projection neurons, cycling progenitors, immature corticofugal projection neurons, immature callosal projection neurons, immature projection neurons, immature interneurons, intermediate progenitor cells, outer radial glia, Cajal-Retzius neurons, and radial glia.

10. The dorsal forebrain organoid of claim 9, comprising about 17%-28% corticofugal projection neurons, about 40%-50% callosal projection neurons, about 4%-7% cycling progenitors, about 2% or less immature interneurons, about 3%-15% immature projection neurons, about 3%-6% intermediate progenitor cells, about 9%-14% radial glia, and/or about 0.5% or less of Cajal-Retzius neurons; and/or
wherein the organoid comprises substantially no astroglia or cycling interneuron precursors.

11.-18. (canceled)

19. The dorsal forebrain organoid of claim 1, wherein the organoid has been cultured for about 6 months or more.

20. The dorsal forebrain organoid of claim 19, wherein the organoid comprises one or more of astroglia, callosal projection neurons, cycling progenitors, immature callosal projection neurons, immature interneurons, immature projection neurons, intermediate progenitor cells, outer radial glia, radial glia, ventral precursors, outer radial glia/astroglia, and cycling interneuron precursors.

21. The dorsal forebrain organoid of claim 20, comprising about 6%-16% astroglia, about 7%-22% callosal projection neurons, about 5%-8% cycling progenitors, about 10%-31% immature interneurons, about 2%-10% immature projection neurons, about 1%-7% intermediate progenitor cells, about 22%-39% radial glia, about 4%-8% ventral precursors, and/or about 4%-8% ventral precursors; and/or
wherein the organoid comprises substantially no corticofugal projection neurons or immature corticofugal projection neurons.

22.-29. (canceled)

30. The dorsal forebrain organoid of claim 1, wherein the organoid has been cultured for at least nine months or more, or
wherein the organoid has been cultured for at least one year or more.

31. (canceled)

32. The dorsal forebrain organoid of claim 1, wherein the dorsal forebrain organoid is a human dorsal forebrain organoid.

33. The dorsal forebrain organoid of claim 1, comprising cells having one or more mutations associated with a neurological disease or condition.

34. A method of producing a dorsal forebrain organoid, comprising obtaining a dorsal forebrain marker-positive organoid by a first step comprising culturing an aggregate of pluripotent stem cells in suspension in the presence of a Wnt signal inhibitor and a TGFβ signal inhibitor, and a second step comprising culturing the dorsal forebrain progenitor marker-positive aggregate in a spinner flask at about 20% oxygen and 5% CO₂.

35. The method of claim 34, wherein the first step is performed for about 18 days.

36. The method of claim 34, wherein the second step is performed for about 35 days or more.

37. The method of claim 36, wherein the obtained dorsal forebrain organoid comprises corticofugal projection neurons, callosal projection neurons, cycling progenitors, immature corticofugal projection neurons, immature callosal projection neurons, immature projection neurons, immature interneurons, intermediate progenitor cells, outer radial glia, Cajal-Retzius neurons, and radial glia.

38. The method of claim 34, wherein the second step is performed for about 162 days or more,
wherein the second step is performed for about 9 months or more, or
wherein the second step is performed for about 1 year or more.

39.-40. (canceled)

41. The method of claim 38, wherein the obtained dorsal forebrain organoid comprises one or more of astroglia, callosal projection neurons, cycling progenitors, immature callosal projection neurons, immature interneurons, immature projection neurons, intermediate progenitor cells, outer radial glia, radial glia, ventral precursors, outer radial glia/astroglia, and cycling interneuron precursors.

42. The method of claim 34, wherein the obtained dorsal forebrain organoid has a core comprising less than 25%, 20%, 15%, 10%, 5%, 1%, or 0.1% apoptotic or hypoxic cells.

43. The method of claim 34, wherein the first step comprises culturing the aggregate of pluripotent stem cells in suspension in the presence of a Wnt signal inhibitor and a TGFβ signal inhibitor for about 18 days and then culturing the aggregate for about 17 days without the presence of a Wnt signal inhibitor and a TGFβ signal inhibitor.

44.-47. (canceled)

48. A method of screening for a candidate neurologically active agent, comprising contacting a dorsal forebrain organoid of claim 1 with a test agent, and assessing changes to the organoid, wherein the test agent is identified as a candidate neurologically active agent when contact with the test agent causes a change to the organoid as compared to a control organoid.

49. (canceled)
